(19) United States
(12) Patent Application Publication
(10) Pub. No.: US 2020/0071367 A1
(43) Pub. Date: Mar. 5, 2020

(54) A NOVEL NERVOUS SYSTEM-SPECIFIC TRANSMEMBRANE PROTEASOME COMPLEX THAT MODULATES NEURONAL SIGNALING THROUGH EXTRACELLULAR SIGNALING VIA BRAIN ACTIVITY PEPTIDES

(71) Applicant: THE JOHNS HOPKINS UNIVERSITY, Baltimore, MD (US)

(72) Inventors: Seth Margolis, Towson, MD (US); Kapil V. Ramachandran, Baltimore, MD (US)

(21) Appl. No.: 16/489,506

(22) PCT Filed: Feb. 28, 2018
(86) PCT No.: PCT/US2018/020173

§ 371 Date: Aug. 28, 2019

Related U.S. Application Data
(63) Continuation-in-part of application No. 15/550,632, filed on Aug. 11, 2017.
(60) Provisional application No. 62/464,446, filed on Feb. 28, 2017.
(60) Provisional application No. 62/470,433, filed on Mar. 13, 2017.

(51) Int. Cl. C07K 14/435; A61K 31/69; A61K 31/24; A61K 31/4015; A61P 25/28

(52) U.S. Cl. CPC C07K 14/435; A61K 31/69; A61K 38/00; A61K 31/4015; A61P 25/28; A61K 31/24

(57) ABSTRACT

The inventors surprisingly found that neural stimulation caused the synthesis and degradation of proteins into peptides which were then secreted into the cell media within minutes of stimulation by a novel neural membrane bound proteasome (NMP). These secreted, activity-induced, proteasomal peptides (SNAPPs) range in size from about 500 Daltons to about 3000 Daltons. Surprisingly none of the peptides appear to be those previously known to have any neuronal function. Moreover, these SNAPPs have stimulatory activity and are heretofore a new class of signaling molecules. The present invention provides methods of modulating NMP function, including in cases of NMP associated disease or disorder of neuronal cells, by stimulating or inhibiting NMP function. The present invention also provides methods for stimulation or enhancing cognitive function using SNAPPs, and methods for treating of NMP related diseases using SNAPPs.

### REFERENCE TO RELATED APPLICATIONS

This application claims the benefit of U.S. Provisional Patent Application No. 62/464,446, filed Feb. 28, 2017, and U.S. Provisional Patent Application No. 62/470,433, filed on Mar. 13, 2017, both of which are hereby incorporated by reference for all purposes as if fully set forth herein.

### STATEMENT OF GOVERNMENTAL INTEREST

This invention was made with government support under grant no. 1R01MH102364, awarded by the National Institutes of Health. The government has certain rights in the invention.

### BACKGROUND OF THE INVENTION

The ability to convert transient stimuli from the extracellular environment into long-term changes in neuronal function is central to an animal's capacity to adapt and learn from its environment. This is mediated through sensory organs which transduce physical and chemical stimuli into precise patterns of neuronal activity that elicit specific changes in the structure and function of the nervous system. Insight into the mechanisms that underlie these activity-dependent changes has been facilitated by the discoveries of many laboratories over the last several decades demonstrating that neurotransmitters released at neuronal synapses drive proteasome dependent protein degradation (J Biol Chem 284, 26655 (2009); Nat Neurosci 6, 231 (2003)). Consistent with a role for neural activity in regulating protein degradation, the proteasome localize to sites of synaptic activity (Nature 441, 1144 (2006)). This regulation is central to the ability of a neuron to appropriately respond to stimuli, as inhibition of protein degradation impairs a host of neuronal functions, ranging from plasticity at the Aplysia sensorimotor synapse to cell migration, neurotransmission, and physiology in the mammalian nervous system (Neuron 32, 1013-1026, 2001; Neuron 52, 239-245, 2006; Cell 89, 115-126, 1997; J Neurosci 26, 11333-11341, 2006) including the maintenance of long-term potentiation, a critical cellular mechanism underlying learning and memory (Neuron 52, 239 (2006); Nat Neurosci 9, 478 (2006)). Moreover, mutations in components of protein degradation machinery cause profound defects in human cognitive function (Biochim Biophys Acta 1843, 13 (2014); Nat Rev Genet 8, 711 (2007)).

However, roles for proteasome function in the nervous system are more complex than they may appear. Proteasome function is required for certain aspects of nervous system function over long timescales (hours to days), such as synaptic remodeling and cell migration (Nat Neurosci 6, 231-242, 2003; Science 302, 1775-1779, 2003). Contrastingly, proteasome function is also required for activity-dependent neuronal processes over very short timescales (seconds to minutes), such as regulating the speed and intensity of neuronal transmission or the maintenance of long-term potentiation (Nature 441, 1144-1148, 2006; Neuroscience 169, 1520-1526, 2010; J Biol Chem 284, 26655-26665, 2009; Learn Mem 15, 335-347, 2008; J Neurosci 26, 4949-4955, 2006; J Neurosci 30, 3157-3166, 2010).

Proteasomes are heterogeneous multisubunit catalytic complexes that consist of a core 20S stacked ring of α/β subunits with a α₇β₇β₇α₇ architecture, and can be associated with 19S or 11S regulatory cap-particles to form a 26S proteasome (Ann. Rev Biochem 65, 801-847, 1996). While the natural behavior of 26S capped proteasomes is to mediate ATP-dependent degradation of ubiquitinated proteins, 20S uncapped proteasomes do not require ubiquitin or ATP for their catalytic function (Biomolecules 4, 862-884, 2014; EMBO J 17, 7151-7160, 1998; Proc Natl Acad Sci U S A 95, Proc Natl Acad Sci U S A 95, 2727-2730 2727-2730, 1998) Recent studies have shown that 20S proteasomes may have key biological functions separate from the canonical 26S ubiquitin-proteasome degradation pathway, particularly in clearing unstructured proteins and in degrading proteins during cellular stress (Ben-Nissan and Sharon, 2014). Despite extensive studies on proteasome function in neuronal signaling, the role of the 20S proteasome in the nervous system has remained unknown.

Critically, the functional studies addressing the role for proteasomes in the nervous system have either failed to discriminate between 20S and 26S proteasomes through the use of pan-proteasome inhibitors such as MG-132 or lactacystin, or have focused on the 26S proteasome through altering the ubiquitination pathway. Despite these and other efforts to understand the role of proteasomes in the nervous system, distinct proteasomes that potentially function independent of their proteostatic role to mediate rapid neuronal signaling have not been discovered. Therefore, we considered that taking an unbiased approach to evaluating proteasomes in the nervous system, without bias for 20S or 26S proteasomes, would provide a means to identify unique proteasomes that could possibly have acute signaling functions.

There exists an unmet need for understanding how protein synthesis and protein degradation cooperate in neurons and whether this cooperation is linked to cognitive function and neurological disease. The use of this information in modulation of cognitive function and neurological disease remains undone.

### SUMMARY OF THE INVENTION

In considering that protein synthesis and protein degradation have independent and opposing effects on the expression level of proteins, it remains to be determined why neuronal activity induces their simultaneous upregulation and co-localization. Indeed, classic studies in the immune system have identified a coordinated and constitutive mechanism of proteasome mediated degradation of newly synthesized proteins, a protein quality control process shown to be critical for proper immune function.

The present inventors hypothesized that in the nervous system coordination of protein synthesis and protein degradation may also alter the turnover of newly synthesized proteins, but unlike the constitutive process in the immune system, may only do so during states of neural activity.

The present inventors' investigation revealed a novel neuronal-specific 20S proteasome complex that was expressed at neuronal plasma membranes and exposed to the extracellular space. It was found that the activity of this novel neural membrane bound proteasome (NMP) converted intracellular proteins into extracellular peptides that rapidly induced neuronal signaling. Specific inhibition of this NMP through a novel membrane-impermeable proteasome inhibitor rapidly attenuated activity-induced neuronal function. These findings identify a new signaling modality in the nervous system and unveil the possibility that the membrane proteasome may be responsible for the previously observed decades of research showing that acute proteasome-mediated effects on nervous system function.

The present inventors monitored the fate of synthesized proteins and found that degradation of proteins by the NMP produced peptides which were directly released into the cell media. Hypothesizing that the NMP may play a role in neuronal activity-dependent mechanisms of nervous system function the inventors found that this release was suppressed when neuronal activity was blocked. Consistent with this finding, the release of these peptides into the media was dramatically enhanced in response to neuronal stimulation. These secreted, neuronal activity-induced, proteasomal peptides (SNAPPs) range in size from about 500 Daltons to about 3000 Daltons. Surprisingly none of these peptides produced by the NMP appear to be those previously known. Moreover, these SNAPPs have stimulatory activity and are heretofore a new class of signaling molecules.

Taken together this discovery defines a new modality of critical neuronal communication through production of biologically meaningful peptides, SNAPPs, that requires the function of a novel neuronal specific transmembrane proteasome, NMP. Changes in the NMP level and possibly activity greatly impact SNAPP production and activity dependent neuronal signaling critical for nervous system function.

In accordance with an embodiment, the present invention provides a method for modulating the NMP in neuronal cells in a subject comprising administering to the subject an effective amount of a NMP stimulator or inhibitor to the subject.

In accordance with another embodiment, the present invention provides a method for modulating an NMP associated disease or disorder of neuronal cells in a subject comprising administering to the subject an effective amount of a NMP stimulator or inhibitor to the subject.

In accordance with a further embodiment, the present invention provides a method for inhibiting neuronal activity or cognitive function in a subject comprising administering to the subject, an effective amount of NMP inhibitor to the subject.

In accordance with a yet another embodiment, the present invention provides a method for stimulating or enhancing neuronal activity or cognitive function in a subject comprising administering to the subject, an effective amount of NMP stimulator to the subject.

In accordance with an embodiment, the present invention provides a method for stimulating or enhancing neuronal activity or cognitive function in a subject comprising administering to the subject, an effective amount of SNAPPs to the subject.

In accordance with a further embodiment, the present invention provides a method for returning neuronal activity or cognitive function to a normal or pre-disease or disorder state in a subject comprising administering to the subject, an effective amount of SNAPPs to the subject.

In accordance with an embodiment, the present invention provides SNAPPs which are covalently linked to one or more biologically active agents.

In accordance with another embodiment, the present invention provides a method for delivery of one or more biologically active agents to activated neurons comprising contacting the activated neurons with SNAPPs which are covalently linked to one or more biologically active agents.

### BRIEF DESCRIPTION OF THE DRAWINGS

FIGS. 1a-1f. 20S proteasome subunits are localized to neuronal plasma membranes. (1a-1f) (left) Western blots of neuronal lysates probed using indicated antibodies. (a-f) (center) Electron micrographs of immunogold labeling (12 nm gold particles) from hippocampal slice preparations using antibodies raised against core catalytic β2 (a, b), β5 (c, d), α2 (e) proteasomal subunits and 19S cap proteasome subunit Rpt5 (f). Representative images shown. White boxes on EM show magnified region (displayed to the right). Several arrows shown corresponding to immunogold label; cytosolic (white); membrane (black). (a-f) (Right) Quantification of gold particles from cytosol (Cyto) and membrane (Mem). N number of micrographs were quantified to get at least 300 gold particles: a) N=49; b) N=47; c) N=43; d) N=54; e) N=54; f) N=92. >300 gold-particles per antibody were counted. Slices were made from two separate 3-month old mice, >20 slices were generated for immuno-EM analysis. Data are presented as mean±SEM.

FIGS. 2a-2f, Neuronal membrane proteasomes are exposed to the extracellular space. (a) Electron micrographs of immunogold labeling (12 nm gold particles) from DIV14 primary mouse cortical neuronal cultures using β2 antibody. Representative images shown. Inset shows magnified region. Ultrastructures: Presynaptic (Pre); Postsynaptic (Post); Microtubules (MT); Synaptic vesicles (SV). Arrows: cytosolic (white); membrane (red-cytosolic face), (yellow-overlaying), (green-extracellular face). (N=84 images, >300 gold-particles. Multiple punches from single culture, >20 slices generated). Quantification to right. (b) Quantification depicted for a subset of gold particles near membranes. Each tick mark represents 2 nm from the plasma membrane (PM). Each dot represents a single gold particle, totals shown above (c) Schematic showing three different approaches to determine whether proteasomes were surface-exposed. (d) Antibody Feeding: Live primary mouse DIV14 cortical neuronal cultures were incubated with antibodies against MAP2, N-terminus of GluR1 (GluR1), or β5 proteasome subunits. Representative images shown, scale bar=10 μm. β5 antibody pre-incubated with the blocking peptide shown below. Quantification of percentage overlap shown (N=2 independent neuronal cultures, n=15 neurons/culture). Significance is calculated between 135 antibody and β5 antibody pre-incubated with blocking peptide. *P<0.01 (two-tailed Student's t-test). (e) Surface biotinylation: Proteins from surface biotinylated DIV14 cortical neurons were precipitated on streptavidin affinity beads and immunoblotted. Representative immunoblots of input lysates (˜3.5% of total, left) and streptavidin pulldown of lysates (Strep) (˜11% of total, right). Quantification is of streptavidin signal normalized to input signal (N=4 independent neuronal cultures). *P<0.01 (one-way ANOVA). (f) Protease Protection: Proteinase K (PK) was applied onto DIV14 cultured cortical neurons for indicated times. Cytosolic (Cyto) and membrane (Mem) fractions were immunoblotted. Quantification is below. Significance for each timepoint against the zero minute timepoint is calculated (N=3 independent neuronal cultures). *P<0.01 (two-tailed Student's t-test). All data are presented as mean±SEM.

FIGS. 3a-3c Neuronal membrane proteasomes are tightly associated with plasma membranes. (a) Primary mouse cortical neuronal cultures at DIV 14 were fractionated into cytosolic (Cyto) and membrane (Mem) components. Membranes were extracted with indicated sequentially increasing concentrations of Digitonin. Samples were analyzed by immunoblotting using antibodies against indicated proteins. Quantification to the right is normalized to input signal levels for each antibody. While 0.25% digitonin extracted cytosolic protein Tubulin, higher concentrations (0.5%, 1.0%) of digitonin were required to extract known hydrophobic proteins such as GluR1. An explanation of percentages loaded on gel is explained in materials and methods. Significance is calculated by comparing signal from the 0.5% digitonin fraction to the 0.25% digitonin fraction for each antibody (N=3 independent neuronal cultures). *P<0.01 (one-way ANOVA). Data are presented as mean±SEM. (b) Proteasome subunits are tightly bound to membranes. Neuronal cultures at DIV14 were fractionated into cytosolic, peripherally-associated (Periph), and tightly-bound (Bound) proteins. Immunoblots of each fraction using indicated antibodies are shown. Quantification to right, data are presented as mean±SEM (N=3 independent neuronal cultures). (c) Cultured neurons at DIV14 were phase separated with TX-114 (TX114). Immunoblots shown using indicated antibodies. TX114-free indicates aqueous phase, and TX114-rich contains the TX-114 phase. Quantification to the right, data are presented as mean±range (N=2 independent neuronal cultures).

FIGS. 4a-4e. Neuronal membrane proteasomes are largely a 20S proteasome and in complex with GPM6 family glycoproteins. (a) Representative immunoblots of proteasomes purified out of neuronal cultures using capped-26S (26S IP) or 20S purification matrices (20S IP). Purification (Pure) was done out of either neuronal cytosol (Cyto) or detergent-extracted neuronal plasma membranes (Mem). (b) Immunoprecipitation with anti-Flag from HEK293 cell lysates previously transfected with plasmids containing Myc/Flag tagged GPM6A and GPM6B, followed by immunoblotting with Myc or proteasome antibodies (α1-7, β2, β5). Inputs (10% of total, left) and immunoprecipitated samples (75% of total, right) are shown. (c) Exogenous expression of GPM6A/B is sufficient to induce surface expression of endogenous proteasomes in HEK293 cells. HEK293 cells were mock transfected (Mock) or transfected with plasmids containing GFP, EphB2, Channelrhodopsin-2 (ChRdp2), GPM6A/B, and GPM6A/B+Myc-tagged β5 (A/B+Myc-β5). Cells were surface biotinylated. Representative immunoblots of input lysates (4% of total, left) and streptavidin pulldowns of lysates (32% of total, right). Quantification shown below is normalized to input signal. β5 western is overexposed in order to see Myc-tagged bands (two arrows, right of immunoblot). Significance is calculated compared to A/B transfected samples (N=3 independent cell cultures and transfections). *P<0.01, one way ANOVA. Data are presented as mean±SEM. (d) Surface-exposed proteasome expression is unique to nervous system tissues. Tissues from P3 mouse were surface biotinylated. Cortex (Ctx), Hippocampus (Hip), Olfactory bulb (Olf), Hind Brain (Brn), Heart (Ht), Lung (Lg), Kidney (Kid), Liver (Lv), Pancreas (Pnc). Representative immunoblots of input lysates (2% of total, left) and streptavidin pulldowns of lysates (4% of total, right). (e) Representative western blots of input lysates (2.5% of total, left) and streptavidin pulldown (7.5% of total, right) of biotinylated proteins following surface biotinylation of mouse cortex tissue dissected from indicated postnatal ages.

FIGS. 5a-5f Neuronal membrane proteasomes degrade intracellular proteins into extracellular peptides (SNAPPs). (a) Purified 20S proteasomes from neuronal cytosol (Cyto) or membrane (Mem) were incubated with the fluorogenic proteasome peptide substrate SUC-LLVY-AMC. Endpoint fluorescence with and without incubation with SDS (0.02%) is quantified. Significance is shown between SDS-treated and untreated samples (N=3 proteasome purifications, independent neuronal cultures). (b) Schematic for collection and purification of extracellular peptides. Media collected from neurons following radiolabeling was subjected to size exclusion purification, with or without Proteinase K (PK). (c) Representative autoradiograph of lysates from cortical neurons previously radiolabeled with 35S methionine/cysteine for 10 minutes in the presence or absence of MG-132. Quantification of signal normalized to vehicle-treated neurons is shown (right). (d) Rapid efflux of radioactive material out of neuronal cultures into media depends upon proteasome function. Media collected from neurons following radiolabeling with or without MG-132 or ATPγS. Liquid scintillation quantification of media at indicated timepoints is shown normalized to MG-132 at 10-minute timepoint; 2 minute timepoint shown separately on bar graph (right) (Media from N=3 independent neuronal cultures). Significance in line graph is shown for MG-132 treated neurons compared to vehicle alone at each time point. (e) Media collected from neurons following radiolabeling was subjected to size exclusion purification, with or without Proteinase K (PK). The percentage of total radioactivity eluting at different sizes is shown (N=3 independent neuronal cultures and purifications). (f) Release of proteasome-derived peptides in the extracellular space correlates with NMP expression. Experiment performed as described in (d); media collected from either DIV7 or DIV8 neurons, with MG-132 (MG-132) or without (Vehicle). (Media of N=2 independent neuronal cultures) *P<0.05 ((a, e, two-tailed Student's t-test, (e) significance of 500<35S Signal<3000Da compared to <500 Da and >3000 Da; (d) One-way ANOVA). Data are mean±SEM (a,c,d,e) or±range (f).

FIGS. 6a-6f Neuronal membrane proteasomes are required for release of extracellular peptides (SNAPPs) and modulate neuronal activity. (a,b) Biotin-epoxomicin does not cross neuronal membranes and covalently modifies proteasome subunits. (a) Neurons treated with biotin-epoxomicin (Bio-Epox) were separated into cytosolic (Cyto) and membrane (Mem) fractions and analyzed by western using streptavidin conjugated to a fluorophore. Immunoblots using indicated antibodies shown below. (b) Immunogold labeling against biotin using streptavidin-Au (black arrows) from neuronal cultures treated with Bio-Epox, with representative images shown. (N=54, obtained from multiple punches of a single neuronal culture, >20 slices generated.) Labeled ultrastructures: Presynaptic regions (Pre), Postsynaptic regions (Post), Microtubules (MT), and synaptic vesicles (SV). Quantification of particles in cytosol and on membrane (right). (c) Specific inhibition of neuronal membrane proteasomes blocks release of extracellular peptides. Media collected from radiolabelled neurons treated with Bio-Epox or without (Vehicle). Liquid scintillation quantification of media at indicated timepoints is shown normalized to Bio-Epox at the 5 minute timepoint; 2 minute timepoint shown separately. Significance is shown for Bio-Epox treated neurons compared to vehicle alone. (N=3 independent neuronal cultures). (d) NMP inhibition modulates speed and intensity of neuronal calcium transients. Bicuculline added (downward black arrowhead) to naïve GCaMP3-encoding neurons. Downward dark blue arrowhead indicates timing of Bio-Epox addition. Representative images (left) and traces of Bicuculline response before and after Bio-Epox addition are plotted (right). Scale bar=40 μM. Quantification of normalized fluorescence intensity (ΔF/F0) measurements of calcium signals over imaging timecourse are shown. (e) Average maximum amplitudes are plotted, and include analysis of calcium signaling after treatment with MG-132. Significance compared to Bicuculline stimulation alone. (f) Box-and-whisker plot of all frequencies observed. *P<0.05, one-way ANOVA (E), two-tailed Student's t-test (B,C). All data are presented as mean±SEM (D-F, N=2 independent replicate cultures, n=24 neurons per culture, with 18 ROIs (regions of interest) analyzed per neuron).

FIGS. 7a-7c. Neuronal membrane proteasome-derived peptides (SNAPPs) are sufficient to induce neuronal signaling. (a) Purified peptides were perfused onto GCaMP3-encoding mouse cortical cultured neurons. Dotted lines indicate time of peptide addition and washout. K+ indicates the timing of 55 mM KCl addition to neurons to determine that they still respond properly at the end of the experiment. Line graph shows increase in fluorescence over baseline during time of peptide addition, a decrease following washout and robust increase with KCl addition. Four sample traces from different neurons are plotted. (b, c) Similar to part (a), cultured neurons were incubated with either Peptides (PK) (peptides were pretreated with P K, PK was removed, and then samples dialyzed to remove small molecules) or with Peptides (MG-132) (peptides purified from cells treated with MG-132). (d-h) Indicated drugs were perfused onto neuronal cultures during the times depicted by the dashed lines. Peptides were subsequently added as indicated and described in (a). Concentrations of drugs: BAPTA (2 μM), Thapsigargin (5 μM), Tetrodotoxin (1 μM), Nifedipine (1 μM), APV (2 μM). (i) Quantification of maximum intensity of change from each condition is plotted. *P<0.01 one-way ANOVA. Data are presented as mean±SEM (N=3 independent replicate cultures, n>15 neurons per treatment, with at least 10 ROIs analyzed per neuron, per condition).

FIG. 8. Proposed theoretical models of NMP association with the plasma membrane. Three models of how proteasomes can associate with plasma membranes are shown above. Extracellular and cytoplasmic sides of the plasma membrane are indicated. Symbol key shown below.

FIG. 9 shows the progression of Alzheimer like symptoms in a mouse model of the disease. Brains from 3 month old mice from J20 AD mouse model (see figure) and wild type mice were treated as in FIG. 1 to isolate the NMP. Samples were run on SDS-PAGE and probed for proteasome, APP and actin.

FIG. 10 depicts DIV16 primary cortical mouse neurons were treated with indicated concentrations of fluorescently labeled Aβ for four hours followed by addition of NMP derived peptides (SNAPPs) for an additional two hours at the indicated concentrations. Neurons were washed and then fixed and stained to assess Aβ1-42 binding. Images to right are magnified from zoomed out image to left. Green fluorescent puncta indicate sites of Aβ1-42 binding. Note high levels of green puncta in the absence of any SNAPP addition as compared to increased levels of SNAPP addition.

FIG. 11 shows primary cultured cortical mouse neurons treated with 1 μM Aβ1-42 or Aβ42-1 for 24 hours and for the final four hours treated with 250 ng of NMP peptides (SNAPPs) or NMP peptides pretreated with proteinase K to eliminate their activity (SNAPPs (PK)). Lysates were prepared for SDS-PAGE and immunoblot analysis using antibodies directed toward phosphorylated Creb (p-Creb(S133)), phosphorylated c-Jun (p-c-Jun (S63)), phosphorylated Erk1/2 (p-Erk1/2 (T202/Y204), cleaved caspase 3 (Cleaved Caspase 3 (Asp 175)), or tubulin loading control (Tuj1). Quantification at right is densitometry analysis of for each western normalized to control lane for the specific treatment.

FIGS. 12A-12D. Neuronal stimulation induces NMP-dependent degradation of newly synthesized proteins into extracellular peptides. (A) Concomitant radiolabelling during neuronal stimulation induces NMP-mediated radiolabeled peptide release. Media collected from neurons concomitantly radiolabeled and treated with control (Con) or KCl stimulation buffer with or without MG-132 or biotin-epoxomicin (Bio-Epox). Liquid scintillation data for media at the indicated time points are shown normalized to control at the 5-minute time point. Data are mean and s.e.m. of n=3 experiments from independent neuronal cultures. Line graph, *p<0.01 (two-way ANOVA) for Control compared to KCl treatment at each time point. Line graph, ∀p<0.01 (two-way ANOVA) for Untreated compared to MG-132 treatment at each time point. Line graph, §p<0.01 (two-way ANOVA) for Untreated compared to Bio-Epox treatment at each time point. (B) Neuronal stimulation induces NMP-mediated degradation of intracellular proteins made during stimulation. Left, representative autoradiograph of lysates from cortical neurons radiolabelled with 35S-methionine/cysteine during either control (C) or KCl (K) stimulation and treated with MG-132 or biotin-epoxomicin (Bio-Epox). Right, quantification of densitometry signal normalized to control alone. Data are mean and s.e.m. of n=3 experiments from independent neuronal cultures. Bar graph, *p<0.01 (two-way ANOVA) compared to control, ∀p<0.01 (one-way ANOVA) for Untreated compared to MG-132 treatment, §p<0.01 (two-way ANOVA) for Untreated compared to Bio-Epox treatment. (C) Neuronal stimulation does not induce NMP-mediated degradation of proteins made prior to stimulation. Left, Representative autoradiograph of lysates from cortical neurons previously radiolabelled and then chased into either control (C) or KCl (K) stimulation buffers for indicated times. Input shows sample collected immediately following labeling. Right, quantification of densitometry signal normalized to control alone. Data are mean and s.e.m. of n=3 experiments from independent neuronal cultures. Statistically significant differences between samples was not observed (two-way ANOVA). (D) Radiolabelling immediately prior to neuronal stimulation does not induce NMP-mediated radiolabeled peptide release. Experiments done as described in (A), note neurons were radiolabelled prior to instead of during stimulation as in (A). Data are mean and s.e.m. of n=3 experiments from independent neuronal cultures. Statistically significant differences between samples was not observed (two-way ANOVA).

FIGS. 13A-13D. Establishment of Markov chain processes to model degradation of nascent chains, folding intermediates, and folded proteins. (A) Overview of model converting protein translation, degradation, and arising fates into Markov decision nodes using experimentally determined radioisotope fate tracing. Experimental timeline to build the Markov chain is shown to the left. Each Markov node is indicated in the top row, as either a free isotope (Met/Cys), Nascent Polypeptide, Folding intermediate, or Folded protein. Probabilities of transitioning between different Markov nodes is indicated by p. Bottom row depicts those paths which can give rise to extracellular isotope release indicated by dashed lines (denoting degradation). Time between steps is shown by τ. (B,C,D) Simulated release curves generated by weighting parameters to bias outcomes. Values for different kinetic parameters based on either calculated or well-established data are shown to the left. Simulated Markov chains (50,000 simulations) to analyze intracellular and extracellular radioisotope composition. The probability for each plot is shown artificially biased towards either Nascent polypeptide (B), Folding intermediate (C), or Folded protein (D). Top, the simulated graphs illustrate the resulting shapes of isotope release curves for a given bias. Each graph represents the proportion of total isotopes at any given second resulting from degradation of nascent polypeptides (Purple), Folding intermediates (Red), or Folded proteins (Blue). Diffusion of free isotope (Grey) was taken into account and constant across conditions. Bottom, the simulated graphs illustrate the resulting shapes of isotope changes inside the cell for a given bias.

FIGS. 14A-14D. Nascent polypeptides are likely the source for NMP-derived extracellular peptides. (A) Parameter space of probabilities of co-translational degradation and folding intermediate degradation to optimize values against experimental data. Optimized values in the indicated parameter space are shown zoomed in to the bottom right. Error minimization for co-translational degradation probability as a 2-dimensional zoomed in representation shown to the bottom left. Note the minimized error for pCTD (probability co-translational degradation) is non-zero and a funnel, compared to pFID (probability folding intermediate degradation). (B) Graph of in silico release data using parameters optimized by minimizing error of probabilities against experimental isotope release data. Calculated release data for untreated (Control) is shown to the left. Calculated release data for neurons stimulated with KCl is shown to right. Insets show zoomed in time-course for the first 300 seconds, similar to experimental release data shown in FIG. 1. Experimental data are shown in black dots, overlaid with simulated release curves. (C) Schematic of experiments with Puromycin. Translating ribosomes shown in grey on mRNA. AUG start site shown just prior to tRNA (small structure with codon recognition loops, in ribosome P site) and growing radioactive polypeptide (growing red line out of translating ribosomes). Puromycin (hexagon) modifies and releases the nascent polypeptide (red) from actively translating ribosomes. (D) Concomitant radiolabelling during neuronal stimulation induces NMP-mediated radiolabeled peptide release that is sensitive to Puromycin treatment. Media collected from neurons concomitantly radiolabeled and treated with control (Con) or KCl stimulation buffer. Puromycin (Puro) or Vehicle added following washout of stimulation and radiolabel. Liquid scintillation data for media at the indicated time points are shown normalized to control at the 5-minute time point. Data are mean and s.e.m. of n=3 experiments from independent neuronal cultures. Line graph, *p<0.01 (two-way ANOVA) compared to control, #p<0.01 (two-way ANOVA) for Untreated compared to Puromycin treatment.

FIGS. 15A-15D. Neuronal stimulation induces NMP-mediated co-translational degradation of ribosome-associated nascent polypeptides. (A) Neuronal stimulation induces NMP-mediated degradation of ribosome-associated polypeptides. Left, top, black line shows timeline over which neurons were treated with either Control (C) or KCl (K) stimulation buffers. Red line shows timeline for radiolabeling, blue line for when pharmacological treatments were introduced. MG-132 and biotin-epoxomicin (Bio-Epox) were added 30 seconds prior to, and for the 30 seconds during radiolabelling. Neurons were lysed in either cycloheximide (CHX) or puromycin (Puro). Left, below, strategy to discriminate ribosome-associated nascent chains. Lysates were layered over a sucrose cushion, and ribosome-nascent chain complexes (RNCs) were pelleted. CHX induces ribosome stalling with tRNA-bound nascent chains (red) still associated with the Ribosome, while Puro dissociates the nascent chain from the Ribosome. Released Puromycylated nascent chains found in supernatant. Right, RNC complexes quantified by liquid scintillation. Graph shows quantification of ribosome scintillation counts, normalized against control alone. Data are mean and s.e.m. of n=3 experiments from independent neuronal cultures. Bar graph, *p<0.01 (two-way ANOVA) for samples compared to controls, #p<0.01 (two-way ANOVA) for samples compared to KCl treatment at each time point. All puromycin treatments were statistically significantly lower than controls, but not significant amongst each other. (B) Experimental strategy to separate tRNA-bound nascent polypeptides from RNCs and full-length proteins. Uncoupled indicate those nascent chains that hydrolyze during separation in first dimension (1D) SDS-PAGE. Lanes are cut out, treated with base at high temperature to hydrolyze the tRNA (dotted lines), and run in a second dimension (2D). Slower migrating signal contains ribosomal proteins, full-length proteins, and those uncoupled from the tRNA in the first dimension. Faster migrating signal contains those nascent chains hydrolyzed from their tRNAs in the base hydrolysis step after the first dimension of SDS-PAGE. (C) Elongating nascent polypeptides during KCl stimulation are degraded by the NMP. Representative autoradiographs of pelleted RNCs from (A) processed by 2D SDS-PAGE. Stimulation condition—either Control (C) or KCl (K) in top right corner, treatment condition—either Vehicle (Veh) or MG-132 in bottom left. Translation inhibitors—either cycloheximide (CHX) or pruomycin (Puro) added during lysis shown above autoradiographs. (D). Ubiquitin immunoblots shown of the same samples in (C).

FIGS. 16A-16D. Quantitative 10-plex mass spectrometry experiment to identify newly synthesized NMP substrates. (A) Overview of mass spectrometry strategy used to enrich and identify NMP targets. Primary cortical neurons were treated with indicated drugs over shown timeline. Bicuculline (Bic), biotin-epoxomicin (Bio-Epox), cycloheximide (CHX). Following protein extraction and trypsinization, biological triplicates for each treatment conditions were labeled with tandem mass tags (TMT tags), indicated by the colors. Peptides were pooled together, fractionated offline using basic reverse-phase liquid chromatography (bRPLC), and then analyzed by MS/MS methods. (B) Scatterplot of normalized log2 bicuculline/Bio-Epox treated compared to both bicuculline alone and bicuculline/Bio-Epox/cycloheximide, versus q-values (p-values after multiple comparisons testing). Representative examples of NMP-targets are highlighted in orange, compared to those targets that do not change by MS analysis with biotin-epoxomicin treatment shown in blue. (C) Heat map of proteins differentially expressed in bicuculline/Bio-Epox treated compared to bicuculline and bicuculline/Bio-Epox/cycloheximide. Coloring indicated percentage of maximum fold change, refer to Methods for details on heat map generation. Top 60 statistically significant targets are shown. (D) Individual targets are shown, with replicates in scatterplot format. Mean and s.e.m are graphed for each condition. ***p<0.001, q<0.1 (two-way ANOVA (p), adjusted for multiple corrections (q) for biotin-epoxomicin (BEp) treatment compared to other samples). NMP targets previously shown to be UPS targets in top row, orange. NMP targets previously uncharacterized with regards to degradation shown in second row, orange. Lower two rows in blue show previously validated activity-dependent UPS targets.

FIGS. 17A-17B. Nascent, not full length, immediate-early gene products are activity-dependent NMP substrates. (A) Immediate-early gene (IEG) products are degraded by the NMP during bicuculline stimulation in a translation-dependent and transcription-independent manner. Primary cortical neurons treated with either MG-132 (MG) or biotin-epoxomicin (BEp) for 10 minutes. Indicated neurons were treated with 1 hour (hr) of bicuculline (Bic). Cycloheximide (CHX) applied only during proteasome inhibition, actinomycin D (ActD) and DMSO (Veh) applied during whole Bicuculline timecourse. Neuronal lysates were immunoblotted using antibodies against indicated proteins. (B) Folded IEG protein is not degraded by the NMP, but is turned over by cytosolic proteasomes. Following 2 hours of bicuculline (Bic) treatment compared to DMSO alone (Veh), neurons were chased into cycloheximide (CHX) for one hour. Neurons treated with either DMSO or bicuculline during CHX Chase. Each set was also treated with either MG-132 (MG), biotin-epoxomicin (BEp), or DMSO (Veh) during chase. Neuronal lysates were immunoblotted using antibodies against indicated proteins. For (A) and (B), protein names in orange classified as NMP targets in mass spectrometry data set (FIG. 5), protein names in blue are not NMP targets based on MS data set. Representative immunoblots shown. Data are mean and s.e.m. of n=3 experiments from independent neuronal cultures.

FIG. 18. Suppression of neuronal activity reduces peptide efflux. Cultured cortical neurons at days in vitro (DIV) 14 were incubated with Tetrodotoxin (TTX—dashed lines, 1 hr) or without (Control—solid line). ³⁵S-methionine/cysteine radiolabel was incorporated for 10 minutes. Radiolabel was washed out, and fresh media +/−TTX was added. Samples were taken at indicated timepoints over a 10 minute timecourse and counted by liquid scintillation. Data are mean and s.e.m. of n=3 experiments from independent neuronal cultures. Line graph, *p<0.01 (Students t-test) for control compared to TTX treatment at each time point.

FIGS. 19A-19F. Neuronal stimulation reduces radiolabel incorporation into proteins in a proteasome dependent manner. (A) Gels for FIGS. 1B and 1C were stained with coomassie dye and dried down onto Whatman filter paper. Note equal loading across conditions. (B) Cortical neurons at Days in vitro 15 were radiolabelled during either ACSF treatment (C) or chemical LTP induction (L) (as described in Materials and methods). MG-132 was added to indicated neurons during stimulation. Autoradiographs quantified by densitometry shown to right. Data are mean and s.e.m. of n=2 experiments from independent neuronal cultures. Bar graph, *p<0.01 (two-way ANOVA) for treatments compared to controls. (C) Neurons were treated with either a Media exchange (M), Glutamate (G), or 5% Fetal Equine Serum (S) and radiolabelled for 10 minutes. Autoradiographs quantified by densitometry shown to right. Data are mean and s.e.m. of n=2 experiments from independent neuronal cultures. Bar graph, *p<0.01 (two-way ANOVA) for treatments compared to controls. (D) Neurons were treated with bicuculline (B) or water (C) for one hour. MG-132 and radiolabel were added during the final 10 minutes of bicuculline stimulation. Autoradiographs quantified by densitometry shown to right. Data are mean and s.e.m. of n=2 experiments from independent neuronal cultures. Bar graph, *p<0.01 (two-way ANOVA) for treatments compared to controls. (E) Neurons stimulated with Control (C) or KCl (K) buffers were separated into Cytosolic (Cyto) and Membrane (Mem) fractions. Proteasomes were purified from each of these samples. Purified proteasomes were incubated for 30 minutes with Suc-LLVY-AMC, a small-molecule proteasome substrate that releases fluorescence when cleaved. Raw fluorescence units are shown. Data are mean and s.e.m. of n=2 experiments from independent neuronal cultures. Bar graph, data were not statistically significantly different across samples (two-way ANOVA). (F) Neurons stimulated with either Control (C) or KCl (K) buffers were incubated with 35S methionine/cysteine radiolabel. Radiolabel was either incorporated at the same time as the stimulation (during), or as soon as the stimulation was washed out into media (following). For following experiment, superscript denotes stimulation condition, red lettering indicates treatment during radiolabelling. Data are mean and s.e.m. of n=3 experiments from independent neuronal cultures. Bar graph, *p<0.01 (two-way ANOVA) for treatments compared to controls.

FIGS. 20A-20C. Optimization of parameters for Markov chain modeling. (A) Probabilities of unfolding were optimized based on previous work calculating average half lives of protein substrates (McShane et al., 2016). Certain protein substrates are much more likely to unfold than others, and while this is highly substrate-dependent, our analyses rely on aggregate data. We first calculated protein half lives based on different values of probability of unfolding and subsequent degradation, plotted above. We then took the approximate half lives of proteins as determined by previous studies that rigorously determine protein half life (McShane et al., 2016). To be on the extremely conservative end of protein half life estimation, we assumed an average and aggregate half life of 20 hours, as indicated by the red line. This was despite an aggregate average of 40-50 hours based on prior work. (B) The error of our predicted in silico Markov chains across the 2D parameter space of probability of background release of radioisotopes (pBackground) versus the probability of loading onto a ribosome (pLoading). This optimization was done under degradation inhibition, to ensure that the observed release is theoretically dominated by the diffusion of radioisotope. The red dot denotes the location of the minimum. The figure on the right is a zoomed in view of the region around the minimum that has up to 10× the error, and indicates that the minimum is very dramatic. The optimal pBackground and pLoading are 0.00017 and 0.0056 respectively. (C) Parameter space of probabilities of co-translational degradation and folding intermediate degradation to optimize values against experimental data. Error minimization for folding intermediate degradation probability (pFID) as a 2-dimensional zoomed in representation shown.

FIG. 21. Parameter space of probabilities of co-translational degradation and folding intermediate degradation to optimize values against experimental KCl stimulation data. The error of our predicted in silico Markov chains across the 2D parameter space of probability of co-translational degradation pCTD versus the probability of folding intermediate degradation pFID. This optimization was carried out under KCl stimulation, and the optimal values of pCTD and pFID were estimated as 0.165 and 0 respectively. The plot on the top right depict the minimum (relative) error achievable given different values of pCTD—indicating a sharp rise in error as pCTD deviates in either direction from the optimized value of 0.165. Similarly, the plot on the bottom right depicts the minimum error achievable given different values of pFID—indicating that the errors steadily increase as pFID deviates from 0.

FIGS. 22A-22C. Neuronal stimulation induces NMP-mediated co-translational degradation of ribosome-associated nascent polypeptides. (A) Ribosome nascent chain (RNC) complexes were pelleted from neurons stimulated with either Control (C) or KCl (K) buffers. MG-132 and Puromycin (Puro) were added to indicated samples. Samples were immunoblotted using antibodies against Ribosomal S6 protein. Immunoblots of inputs are shown above those for pelleted RNC (Ribo pellet). (B) Pelleted RNCs from HEK293 cells, treated with Vehicle or MG-132. Samples analyzed by liquid scintillation. Scintillation counts normalized to vehicle-treated samples shown, average of n=3 biological replicates plotted as mean and s.e.m. (C) Pelleted RNCs from Control or KCl stimulated neurons treated with or without vehicle, MG-132, or biotin-epoxomicin (Bio-Epox).

FIGS. 23A-23B.: Immediate-early gene products are activity-dependent NMP substrates. (A) Primary cortical neurons at DIV15 treated with bicuculline (Bic) for indicated times. Treatment conditions above: DMSO (Veh), cycloheximide (CHX), actinomycin D (ActD), Tetrodotoxin (TTX). Treatments applied as indicated. Neuronal lysates were immunoblotted using antibodies against indicated proteins. Protein names in orange classified as NMP targets in mass spectrometry data set (FIG. 3), protein names in blue not NMP targets based on MS data set. Representative immunoblots shown. (B) Primary cortical neurons at DIV15 treated with bicuculline (Bic) for 1 hour. Treatment conditions above: DMSO (Veh), cycloheximide (CHX), actinomycin D (ActD), Tetrodotoxin (TTX). MG-132 (MG) or biotin-epoxomicin (BEp) applied for final 10 minutes of 1 hour Bicuculline stimulation. Neuronal lysates were immunoblotted using antibodies against indicated proteins. Protein names in orange classified as NMP targets in mass spectrometry data set (FIG. 3), protein names in blue not NMP targets based on MS data set. Representative immunoblots shown. Significance table presented in supplement. For (A) and (B), protein names in orange classified as NMP targets in mass spectrometry data set (FIG. 5), protein names in blue are not NMP targets based on MS data set. Representative immunoblots shown. Data are mean and s.e.m. of n=3 experiments from independent neuronal cultures.

### DETAILED DESCRIPTION OF THE INVENTION

Proteasomes are ubiquitously expressed large multi-subunit catalytic complexes, generally characterized by a uniform cytoplasmic and nuclear distribution. The present inventors have now identified a nervous system-specific proteasome that is bound to the plasma membrane and exposed to the extracellular space. While it is unclear how these proteasomes bind to and orient themselves within neuronal plasma membranes, it has been known for decades through in vitro studies that proteasomes can orient perpendicularly to membranes specifically enriched in phosphatidylinositol (PI), a key signaling phospholipid that is notably elevated in the nervous system over other tissues.

The present inventors have discovered the presence of a 20S proteasome that is tightly associated with the neuronal plasma membrane and exposed to the extracellular space. In this capacity, it can degrade intracellular proteins into bioactive extracellular peptides that induce calcium signaling through NMDA receptors. Without reliance on any particular theory, the preferred model (discussed further below) based on these data are that a 20S proteasome complex is coupled to the plasma membrane by GPM6 glycoproteins, and that the extracellular peptides generated are the means by which the NMP acutely regulates neuronal function.

Identification of the GPM6 glycoprotein family as proteins that interact with proteasomes and are sufficient to induce the expression of proteasomes at the plasma membrane provides some insight into how proteasomes, as hydrophilic protein complexes, could interact so tightly with the hydrophobic plasma membrane. However, we noticed that the magnitude to which GPM6-induced membrane proteasome expression in heterologous cells did not match the magnitude of endogenous membrane proteasome expression in neurons. This suggests that there may in fact be other proteins that mediate the interaction of the NMP with the membrane, an area being actively investigated.

It is presently thought that the GPM6 glycoproteins may form a protein pore, perhaps through oligomeric interactions, which have been proposed previously^{35,44}. In the right conformation, proteasomes binding to pore-containing membrane proteins could give proteasomes a hydrophilic binding surface to the hydrophobic plasma membrane, allowing the proteasome to gain access to the extracellular space. We propose a few models for how GPM6 proteins, or other membrane tethers may localize the proteasome to the plasma membrane (FIG. 8). In each case, we posited that 1) proteasomes must be located at plasma membranes, 2) proteasomes were in some fashion bound to auxiliary membrane proteins such as GPM6, and 3) proteasomes must be able to degrade proteins from the intracellular to the extracellular space. Model 1—Cytoplasmic docking: In this model, a proteasome located at the plasma membrane would be docked on or tethered to auxiliary membrane proteins on the cytoplasmic side of the membrane. Degraded proteins would be shed through a peptide pore formed by the auxiliary proteins. Model 2—Extracellular docking: In this model, a proteasome located at the plasma membrane would be docked on or tethered to auxiliary membrane proteins on the extracellular side of the membrane. Proteins would be delivered through a protein pore formed by the auxiliary proteins. Model 3—Intramembrane docking: In this model, a proteasome located at the plasma membrane would be tethered or anchored to auxiliary membrane proteins within the lipid bilayer. The cell biological conundrum of how a proteasome can interact with the plasma membrane may be the most significant question to address in order to gain a deeper understanding of NMP function. Because antibody feeding and protease protection require that large molecules gain access to the proteasome, we posit that model 1 is less likely, and either model 2 or model 3 will prevail. While we find these models most consistent with our data, we certainly do not preclude other potential models. Ultimately, the nature of this seemingly transmembrane complex can only be validated by a structural approach.

The inventors made significant attempts to identify NMP interacting partners in an effort to determine whether the NMP was capped by the 19S, 11S, or PA200 subunits. Our data likely preclude the presence of the canonical 19S proteasome cap, or regulatory caps such as 11S or PA200^{4,45,46}. While we identified a few 19S subunits co-fractionating with the NMP by mass spectrometry, we could not identify significant amount of key 19S subunits Rpt5 or S2. We also made the intriguing observation that immunoproteasome subunit PSMB8 uniquely co-fractionated with the NMP. Our finding that the NMP is likely a 20S core proteasome lacking the 19S cap is significant for two primary reasons. First, while a few functions for 20S proteasomes have been ascribed, their function independent of the 19S cap largely remains a mystery, especially in the nervous system⁴⁶. Second, significant implications come from the idea that 20S proteasomes are primarily tasked with clearing misfolded or unstructured proteins^{4,47,48}. A large source of disordered or unfolded proteins is derived from failed products of protein translation and misfolded or improperly folded proteins. These end-products of proteotoxic stress are hallmarks of many neurodegenerative disorders^{49,50}, a fact which places the NMP at the heart of various disease states.

The present inventors have found that neuronal activity does not simply promote global protein degradation, but rather, it promotes protein degradation exclusively of newly synthesized proteins through the NMP for the express purpose of generating a new class of signaling molecules, SNAPPs.

Unconventional secretion pathways have been implicated in release of cellular protein cargos^{51,52}. Moreover, many groups have demonstrated that inhibition of ubiquitin-dependent proteasome function affects synaptic signaling and transmission. The data of the present invention support a role for the existence of a specialized neuronal membrane proteasome that mediates neuronal function by “inside-out” signaling through the production of extracellular proteasome-derived peptides. While it remains possible, we have not detected any role for secretion pathways or ubiquitin in the release of these peptides (Ramachandran and Margolis, unpublished data).

The SNAPPs of the present invention are a new modality for neuronal communication. In the release experiments described herein, we show that there is some peptide release under non-stimulating conditions that is inhibited by MG-132, a known proteasomal inhibitor. It is thought that this is due to baseline spontaneous network activity causing some baseline degradation of proteins by the NMP, leading to peptides being released into the media. These peptides are different than SNAPPs, as they do not possess the same signaling capacity as SNAPPs.

SNAPPs, which when purified, rapidly and robustly stimulate neurons. Pharmacological dissection of the downstream pathways of peptide signaling revealed that NMP-derived peptides act in part by modulating NMDARs. The signaling through NMDARs only makes up ˜50% of the total activity of the peptides. Other possible targets include: 1) Peptides interact with major histocompatibility immune complexes (MHC) that have recently been shown to play key roles in developmental and experience-dependent mechanisms in the nervous system^{53,54}; 2) peptides modulate metabotropic ion channels, thereby altering calcium-mediated signaling; and/or 3) peptides signal to neuronal or non-neuronal cells such as glial cells through yet to be identified receptors.

It is well-established that NMDARs are critical for neuronal activity-dependent signaling relevant to learning and memory⁵⁵⁻⁵⁷. Given that cytosolic proteasomes have been shown to be regulated by neuronal activity, it is thought that the NMP and the resulting extracellular peptides are also modulated by changes in neuronal activity. It is also unclear how this signaling is specified within the brain, but we postulate that it relies on how the NMP recognizes and targets proteins for degradation. Therefore, it will be critical to identify not only the sequences of the peptides, but also the substrates from which they are derived. These insights into substrate identity and targeting will reveal how the NMP functions, but may begin to link proteostatic failure under pathological conditions to NMP dysfunction.

Of note in some aspects of the present invention, is the role for phosphorylated CamKII in NMP expression. This is particularly intriguing given the role for phosphorylated CaMKII in serving as a scaffold for recruiting the proteasome into dendritic spines, and additionally for its long-known and well-studied role in learning and memory.

The same groups that have demonstrated the role for CaMKII in proteasome recruitment to spines have also shown that rapid inhibition of the proteasome has profound effects on synaptic signaling and transmission. These effects range from changes in transmission at the Drosophila neuromuscular synapse, regulation of activity-dependent spine dynamics, and an essential role in maintenance of LTP. In accordance with the inventive compositions and methods, we see a similar rapid and acute role for the proteasome in mediating SNAPP release (data not shown). It is important to note that pharmacological inhibitors used in previous studies take a substantially longer time to achieve functional inhibition of the cytosolic proteasome, according to data from groups studying the kinetics of proteasome inhibitors in neurons. Given the present findings, it is thought that at least some of the effects on synaptic transmission and function demonstrated by older studies may be due to inhibition of the neuronal membrane proteasome first reported in this study, and not of the cytosolic proteasome.

As used herein, the term “Neuronal Membrane Proteasome (NMP)” means a neuronal-specific 20S proteasome complex that was expressed at neuronal plasma membranes and exposed to the extracellular space. The NMP is unique to the nervous system and produces SNAPPs into the extracellular space.

As used herein, the analysis of proteins which are located on the plasma membrane surface of the neuronal cell, can be performed using many different means known in the art. In an embodiment, the plasma membrane fraction is isolated from neurons by lysing them in either a sucrose buffer or hypotonic lysis buffer. Nuclei were pelleted, and the supernatant containing plasma membranes was then pelleted at high RPM. Once the supernatant (cytosolic fraction) was set aside, the pellet was washed 2× with lysis buffer, and then resuspended in lysis buffer with indicated concentrations of detergent. Following a 15-minute incubation in the buffer, samples were spun down. This was repeated for all indicated concentrations of detergent. Membrane association was determined by classic methods of sodium carbonate extraction. The proteins were visualized by SDS-PAGE methods. Other methods can be used.

As used herein the 20S core proteins associated with the NMP can be identified and analyzed through the use of an antibodies that detect β2, anti-α1-7 proteasome subunit, anti-α5 proteasome subunit, anti-β1 proteasome subunit, anti-β2,5 subunit, anti-β2 proteasome subunit, and anti-Rpt5 proteasome subunit, for example. Other method for identification are known in the art, and include, for example, surface biotinylation methods and mass spectrometry.

In accordance with an embodiment, the present invention provides a composition comprising one or more SNAPPs.

In accordance with an embodiment, the present invention provides a composition comprising secreted, neuronal activity-induced, proteasomal peptides (SNAPPs), in an effective amount, for use in stimulating or enhancing neuronal activity or cognitive function in a subject.

In some embodiments, the SNAPPs have a molecular weight between 500 to 3000 Daltons.

In some embodiments, the SNAPPs are derived from a neuron selected from the group consisting of cortical, hippocampal, cerebellar, motor, sensory,

In some embodiments, the SNAPPs comprise at least one detectable moiety as an imaging agent.

In some embodiments, the SNAPPs comprise at least one detectable moiety as a radionuclide.

In some embodiments, the at least one detectable moiety is covalently attached to the SNAPPs via a biotinylated linker molecule.

In some embodiments, the subject is suffering from Alzheimer's disease or dementia.

In some embodiments, the composition further comprises an effective amount of at least one additional biologically active agent.

As used herein, the term “SNAPP” means proteins and peptides which are secreted extracellularly by a novel neural membrane bound proteasome (NMP) as the result of neural stimulation. Typically, these SNAPPs are secreted extracellularly within a few seconds to minutes after neural stimulation. These SNAPPs range in size from about 500 Daltons to about 3000 Daltons.

The term, “amino acid” includes the residues of the natural α-amino acids (e.g., Ala, Arg, Asn, Asp, Cys, Glu, Gln, Gly, His, Lys, Ile, Leu, Met, Phe, Pro, Ser, Thr, Trp, Tyr, and Val) in D or L form, as well as (3-amino acids, synthetic and unnatural amino acids. Many types of amino acid residues are useful in the adipokine polypeptides and the invention is not limited to natural, genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC.

The term, “peptide,” or “oligopeptide,” as used herein, includes a sequence of from four to sixteen amino acid residues in which the α-carboxyl group of one amino acid is joined by an amide bond to the main chain (α- or β-) amino group of the adjacent amino acid. In some embodiments, peptides provided herein for use in the described and claimed methods and compositions can be cyclic.

The term “imaging agent,” is known in the art. As used herein, the one or more imaging agents can be any small molecule or radionuclide which is capable of being detected. Typically, the imaging agents are covalently linked to the SNAPPs using any known methods in the art. Examples include use of a linker molecule. Other examples include biotinylation and biotin linked dyes.

In accordance with some embodiments the imaging agent is a fluorescent dye. The dyes may be emitters in the visible or near-infrared (NIR) spectrum. Known dyes useful in the present invention include carbocyanine, indocarbocyanine, oxacarbocyanine, thiiicarbocyanine and merocyanine, polymethine, coumarine, rhodamine, xanthene, fluorescein, boron-dipyrromethane (BODIPY), CyS, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18π-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4a-diaza-s-indacene) and sharp fluorescence with high quantum yield and excellent thermal and photochemical stability.

Other imaging agents which can be attached to the SNAPPs of the present invention include PET and SPECT imaging agents. The most widely used agents include branched chelating agents such as di-ethylene tri-amine penta-acetic acid (DTPA), 1,4,7,10-tetra-azacyclododecane-1,4,7,10-tetraacetic acid (DOTA) and their analogs. Chelating agents, such as di-amine dithiols, activated mercaptoacetyl-glycyl-glycyl-gylcine (MAG3), and hydrazidonicotinamide (HYNIC), are able to chelate metals like ^{99m}Tc and ¹⁸⁶Re. Instead of using chelating agents, a prosthetic group such as N-succinimidyl-4-¹⁸F-fluorobenzoate (¹⁸F-SFB) is necessary for labeling peptides with ¹⁸F. In accordance with a preferred embodiment, the chelating agent is DOTA.

In accordance with some embodiments, the present invention provides one or more SNAPPs wherein the imaging agent comprises a metal isotope suitable for imaging. Examples of isotopes useful in the present invention include Tc-94m, Tc-99m, In-111, Ga-67, Ga-68, Y-86, Y-90, Lu-177, Re-186, Re-188, Cu-64, Cu-67, Co-55, Co-57, Sc-47, Ac-225, Bi-213, Bi-212, Pb-212, Sm-153, Ho-166, or Dy-i66.

In accordance with some embodiments, the present invention provides a SNAPP wherein the reporter portion comprises ¹¹¹In labeled DOTA which is known to be suitable for use in SPECT imaging.

In accordance with some other embodiments, the present invention provides SNAPPs wherein the imaging agent comprises Gd³⁺ labeled DOTA which is known to be suitable for use in MR imaging. It is understood by those of ordinary skill in the art that other suitable radioisotopes can be substituted for ¹¹¹In and Gd³⁺ disclosed herein.

In some embodiments, the present invention provides methods for detecting neuronal activity using voltage-sensitive dye, whose optical properties change during changes in electrical activity of neuronal cells. The spatial resolution achieved by this technique is near the single cell level. For example, researchers have used the voltage-sensitive dye merocyanine oxazolone to map cortical function in a monkey model. Blasdel, G. G. and Salama, G., “Voltage Sensitive Dyes Reveal a Modular Organization Monkey Striate Cortex,” Nature 321:579-585, 1986. However, the use of these kinds of dyes would pose too great a risk for use in vivo in view of their toxicity.

It will be understood by those of ordinary skill in the art that the SNAPPs of the present invention have the ability to bind activated neurons, and therefore they can be used as targeting molecules for other therapies. For example, SNAPPs can be conjugated with another small molecule, or biologically active agent, including, drugs, antibodies and the like. In accordance with some embodiments, the SNAPPs can be conjugated or linked with compounds which stimulate or inhibit neuronal activity, or which have some other pharmacological effect.

As used herein, the term “biologically active agent” include any compound, biologics for treating brain-related diseases, e.g. drugs, inhibitors, and proteins. An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc.

In accordance with some embodiments, the SNAPPs can be conjugated or linked with compounds which stimulate or inhibit neuronal activity. Examples of such classes of compounds include, but are not limited to, cholinergic agonists and antagonists, opiate agonists and antagonists, muscarinic agonists and antagonists, GABAergic agonists and antagonists, parasympathomimetics, sympathomimetics, adrenergic agonists and antagonists, general anesthetics, such as inhalation anesthetics, halogenated inhalation anesthetics, intravenous anesthetics, barbiturates, benzodiazepines, antidepressants, heterocyclic antidepressants, monoamine oxidase inhibitors selective serotonin re-uptake inhibitors tricyclic antidepressants, antimanics, anti-psychotics, phenothiazine antipsychotics, anxiolytics, calcium channel blockers, and anti-Parkinson's agents such as bromocriptine, levodopa, carbidopa, and pergolide.

It is understood by those of ordinary skill in the art that the compounds and/or imaging agents can be attached to the SNAPPs by use of linker molecules. For instance linking groups having alkyl, aryl, combination of alkyl and aryl, or alkyl and aryl groups having heteroatoms may be present. For example, the linker can be a C₁-C₂₀ alkyl, C₂-C₂₀ alkenyl, C₂-C₂₀ alkynyl, C₁-C₂₀ hydroxyalkyl, C₁-C₂₀ alkoxy, C₁-C₂₀ alkoxy C₁-C₂₀ alkyl, C₁-C₂₀ alkylamino, di-C₁-C₂₀ alkylamino, C₁-C₂₀ dialkylamino C₁-C₂₀ alkyl, C₁-C₂₀ thioalkyl, C₂-C₂₀ thioalkenyl, C₂-C₂₀ thioalkynyl, C₆-C₂₂ aryloxy, C₆-C₂₂ arylamino C₂-C₂₀ acyloxy, C₂-C₂₀ thioacyl, C₁-C₂₀ amido, and C₁-C₂₀ sulphonamido.

Compounds are assembled by reactions between different components, to form linkages such as ureas (—NRC(O)NR—), thioureas (—NRC(S)NR—), amides (—C(O)NR— or —NRC(O)—), or esters (—C(O)O— or —OC(O)—). Urea linkages may be readily prepared by reaction between an amine and an isocyanate, or between an amine and an activated carbonamide (—NRC(O)—). Thioureas may be readily prepared from reaction of an amine with an isothiocyanate. Amides (—C(O)NR— or —NRC(O)—) may be readily prepared by reactions between amines and activated carboxylic acids or esters, such as an acyl halide or N-hydroxysuccinimide ester. Carboxylic acids may also be activated in situ, for example, with a coupling reagent, such as a carbodiimide, or carbonyldiimidazole (CDI). Esters may be formed by reaction between alcohols and activated carboxylic acids. Triazoles are readily prepared by reaction between an azide and an alkyne, optionally in the presence of a copper (Cu) catalyst.

Protecting groups may be used, if necessary, to protect reactive groups while the compounds are being assembled. Suitable protecting groups, and their removal, will be readily available to one of ordinary skill in the art.

In this way, the compounds may be easily prepared from individual building blocks, such as amines, carboxylic acids, and amino acids.

It is contemplated that any of the SNAPPs of the present invention described above can also encompass a pharmaceutical composition comprising the SNAPPs and a pharmaceutically acceptable carrier.

With respect to the SNAPPs described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

The choice of carrier will be determined, in part, by the particular SNAPP composition, as well as by the particular method used to administer the composition. Accordingly, there are a variety of suitable formulations of the pharmaceutical SNAPP composition of the invention. The following formulations for parenteral, subcutaneous, intravenous, intramuscular, intraarterial, intrathecal and interperitoneal administration are exemplary, and are in no way limiting. More than one route can be used to administer the compositions of the present invention, and in certain instances, a particular route can provide a more immediate and more effective response than another route.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of the SNAPPs of the present invention that is administered should be sufficient to effectively target the cell, or population of cells in vivo, such that the stimulation of the neuronal cells can be detected, in the subject over a reasonable time frame. The dose will be determined by the efficacy of the particular SNAPP formulation and the location of the target population of neuronal cells in the subject, as well as the body weight of the subject to be treated.

The dose of the SNAPPs of the present invention also will be determined by the existence, nature and extent of any adverse side effects that might accompany the administration of a particular SNAPP. Typically, an attending physician will decide the dosage of the SNAPPs with which to treat each individual subject, taking into consideration a variety of factors, such as age, body weight, general health, diet, sex, compound to be administered, route of administration, and the severity of the condition being treated. By way of example, and not intending to limit the invention, the dose of the SNAPPs of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the dose of the SNAPPs of the present invention can be at a concentration from about 1 nM to about 10,000 nM, preferably from about 10 nM to about 5,000 nM, more preferably from about 100 nM to about 500 nM.

In accordance with another embodiment, the present invention provides a method for identifying activated neurons in vitro comprising: a) providing a plurality of in vitro cultures comprising a plurality of neurons in a growth medium; b) stimulating at least one or more of the cultures with a stimulant; c) removing the growth medium of the plurality of in vitro cultures; d) fixing the plurality of in vitro cultures; e) staining the plurality of in vitro cultures with at least one or more SNAPP compositions as described herein; f) quantifying the detectable moiety of the compositions of e) using imaging and/or radiography; g) identifying the activated neurons as those neurons from stimulated in vitro cultures which have a significantly increased amount of detectable signal from the detectable moiety compared to the amount of detectable signal in neurons from in vitro cultures which were not stimulated.

In accordance with another embodiment, the present invention provides a method for identifying activated neurons in vivo comprising: a) administering to the neuronal tissue of a mammal an effective amount of at least one or more SNAPP compositions as described herein, wherein the imaging agent is a SPECT or PET, or magnetic resonance imaging agent; b) imaging the neuronal tissue of the mammal; and c) identifying the activated neurons as those neurons which have a significantly increased amount of detectable signal from the detectable moiety compared to the amount of detectable signal from other neurons in the tissue.

In accordance with a further embodiment, the present invention provides a method for screening for compounds which stimulate NMP and subsequent production of secreted neuronal-activity induced proteasomal peptides (SNAPPs) comprising the steps of: a) administering to a subject a test compound for a period of time sufficient to stimulate NMP and allow production of SNAPPs in the neurons of the subject; b) providing a negative control by administering to at least a second subject for a period of time sufficient with a carrier or vehicle which will not stimulate NMP mediated production of SNAPPs in the neurons of the second subject; c) obtaining a biological sample from a) and b) and performing an isolation step to purify the SNAPPs from the biological samples of b) and c); d) quantifying the amount of SNAPPs isolated in e) from the biological samples of a) and b); and e) determining that the test compound is a stimulator of NMP mediated SNAPP production when the quantity of SNAPPs isolated from the biological samples of a) are significantly increased when compared with the amount of SNAPPs isolated from the biological samples of b).

In accordance with another embodiment, the present invention provides a method for identifying activated neurons in vivo comprising: a) administering to the neuronal tissue of a mammal an effective amount of at least one or more SNAPP compositions as described herein, wherein the imaging agent is a SPECT or PET, or magnetic resonance imaging agent; b) imaging the neuronal tissue of the mammal; and c) identifying the activated neurons as those neurons which have a significantly increased amount of detectable signal from the detectable moiety compared to the amount of detectable signal from other neurons in the tissue.

In some embodiments the present invention employs an electromagnetic radiation (emr) source for uniformly illuminating an area of neurons of interest, and an optical detector capable of detecting and acquiring data relating to one or more optical properties of an area of interest. In a simple form, the apparatus of the present invention may include an optical fiber operably connected to an emr source that illuminates tissue or neuronal cultures in vitro, and another optical fiber operably connected to an optical detector, such as a photodiode, that detects one or more optical properties of the illuminated tissue. The detector is used to obtain control data representing the “normal” or “background” optical properties of an area of interest, and then to obtain subsequent data representing the optical properties of an area of interest during neuronal activity, e.g., stimulation of neuronal tissue, or during a monitoring interval. The subsequent data is compared to the control data to identify changes in optical properties representative of neuronal activity. According to a preferred embodiment, the control, subsequent and comparison data are presented in a visual format as images.

In some embodiments, the present invention provides methods for optically imaging neuronal tissue and the physiological events associated with neuronal activity. The methods of the present invention may be used for optically imaging and mapping functional neuronal activity, differentiating neuronal tissue from non-neuronal tissue, identifying and spatially locating dysfunctional neuronal tissue, and monitoring neuronal tissue to assess viability, function and the like.

Numerous devices for acquiring, processing and displaying data representative of one or more optical properties of an area of interest can be employed. One preferred device is a video camera that acquires control and subsequent images of an area of interest that can be compared to identify areas of neuronal activity or dysfunction. Examination of images provides precise spatial location of areas of neuronal activity or dysfunction. Apparatus suitable for obtaining such images have been described in the patents incorporated herein by reference and are more fully described below. For most surgical and diagnostic uses, the optical detector preferably provides images having a high degree of spatial resolution at a magnification sufficient to detect single neuronal cells or nerve fiber bundles. Several images are preferably acquired over a predetermined time period and combined, such as by averaging, to provide control and subsequent images for comparison.

In some embodiments the video camera is a Charge Coupled Device (CCD). A CCD is a type of optical detector that utilizes a photo-sensitive silicon chip in place of a pickup tube in a video camera.

Various data processing techniques may be advantageously used to assess the data collected in accordance with the present invention. Comparison data may be assessed or presented in a variety of formats. Processing may include averaging or otherwise combining a plurality of data sets to produce control, subsequent or comparison data sets. Images are preferably converted from an analog to a digital form for processing, and back to an analog form for display.

Data processing may also include amplification of certain signals or portions of a data set (e.g., areas of an image) to enhance the contrast seen in data set comparisons, and to thereby identify areas of neuronal activity and/or dysfunction with a high degree of spatial resolution. For example, according to one embodiment, images are processed using a transformation in which image pixel brightness values are remapped to cover a broader dynamic range of values. A “low” value may be selected and mapped to zero, with all pixel brightness values at or below the low value set to zero, and a “high” value may be selected and mapped to a selected value, with all pixel brightness values at or above the high value mapped to the high value. Pixels having an intermediate brightness value, representing the dynamic changes in brightness indicative of neuronal activity, may be mapped to linearly or logarithmically increasing brightness values. This type of processing manipulation is frequently referred to as a “histogram stretch” and can be used according to the present invention to enhance the contrast of data sets, such as images, representing changes in neuronal activity.

In accordance with another embodiment, the present invention provides a method for making SNAPPs comprising the steps of: a) providing an in vitro culture of a plurality of neurons in a growth medium; b) stimulating the neurons for a period of time sufficient to allow secretion of SNAPPs into the growth medium; c) removing at least a portion of the growth medium containing the SNAPPs.

The term “neuron” is used herein to denote a cell that arises from neuroepithelial cell precursors. Mature neurons (i.e., fully differentiated cells from an adult) display several specific antigenic markers.

The term “neuroepithelium” is used herein to denote cells and tissues that arise from the neural epithelium during development; such cells include retinal cells, diencephalon cells and midbrain cells. Neuroepithelium is also defined as neuroectoderm, and more specifically as ectoderm on the dorsal surface of the early vertebrate embryo that gives rise to the cells (neurons and glia) of the nervous system.

As used herein, the term “neuron” means neuronal cells derived from the central nervous system of a subject, including, for example, the brain, spinal cord, as well as the peripheral nervous system, including, for example, sensory and motor neurons. Areas of the brain where these neurons can originate from include, but are not limited to, Cortex (Ctx), Hippocampus (Hip), Olfactory bulb (Olf), Hind Brain (Brn), for example. Neurons can also be cells derived from induced pluripotent stem cell (iPSC) cultures.

The cell culture systems and methods used in the present invention may be used in conjunction with any glass surface (including, for instance, coverslips) that has been coated with an attachment-enhancing substance, such as poly-lysine, Matrigel, laminin, polyornithine, gelatin and/or fibronectin. Feeder cell layers, such as glial feeder layers or embryonic fibroblast feeder layers, may also find use within the methods and compositions provided herein.

Neuronal cells used in the present invention can be placed into any known culture medium capable of supporting cell growth, including MEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. A particularly preferable medium for cells is a mixture of Neurobasal and B-27 (catalog #21103049 and 17504044 respectively, Life Technologies, Gaithersburg, Md.).

Conditions for culturing should be close to physiological conditions. The pH of the culture media should be close to physiological pH, preferably between pH 6-8, more preferably close to pH 7, even more particularly about pH 7.4. Cells should be cultured at a temperature close to physiological temperature, preferably between 30° C.-40° C., more preferably between 32° C.-38° C., and most preferably between 35° C.-37° C.

Neuronal cells can be grown in suspension or on a fixed substrate. In the case of propagating (or splitting) suspension cells, flasks are shaken well and the neurospheres allowed to settle on the bottom corner of the flask. The spheres are then transferred to a 50 ml centrifuge tube and centrifuged at low speed. The medium is aspirated, the cells resuspended in a small amount of medium with growth factor, and the cells mechanically dissociated and resuspended in separate aliquots of media.

Cell suspensions in culture medium are supplemented with any growth factor which allows for the proliferation of progenitor cells and seeded in any receptacle capable of sustaining cells, though as set out above, preferably in culture flasks or roller bottles. Cells typically proliferate within 3-4 days in a 37° C. incubator, and proliferation can be reinitiated at any time after that by dissociation of the cells and resuspension in fresh medium containing growth factors.

As used herein, the term “stimulation” means the activation or firing of the neuron when the neuron is stimulated by pressure, heat, light, or chemical information from other cells. The type of stimulation necessary to produce firing depends on the type of neuron. The cytosol inside a neuron is separated from that outside by a polarized cell membrane that contains electrically charged particles known as ions. When a neuron is sufficiently stimulated to reach the neural threshold (a level of stimulation below which the cell does not fire), depolarization, or a change in cell potential, occurs.

In accordance with some embodiments, neurons which produce SNAPPs can be stimulated by the use of a depolarizing buffer. Examples of such buffers include, but are not limited to physiological buffers containing high concentration of KCl (60 mM to 150 mM or more), and can also include additional Ca⁺⁺ ions (10-20 mM). Other such depolarizing buffers include glutamate or bicuculine and others.

Removal of cell growth medium from cell cultures which have been stimulated can be performed using any known means in the art, e.g., pipetting, filtration, etc.

In accordance with an embodiment, the present invention provides a method for inhibiting secreted neuronal-activity induced proteasomal peptides (SNAPPs) in a neuronal cell or population of cells comprising contacting the cell or population of cells with an effective amount of at least one proteasomal inhibitor for a time sufficient to inhibit secretion of SNAPPs.

In some embodiments, the proteasomal inhibitor can be one known in the art. For example, compounds such as Epoxomicin, Lactacystin, Bortezomib, MG-132, Carfilzomib, MLN9708, Ixazomib, PI-1840, ONX-0914, Oprozomib, CEP-18770, and Gabexate Mesylate are known proteasomal inhibitors.

In accordance with a further embodiment, the present invention provides a method for screening for compounds which stimulate secretion of secreted neuronal-activity induced proteasomal peptides (SNAPPs) comprising the steps of: a) providing a plurality of in vitro cultures comprising a plurality of neurons in a growth medium; b) providing one or more test cultures by contacting the neurons of at least a first culture with a test compound for a period of time sufficient to allow secretion of SNAPPs into the growth medium; c) providing a negative control by contacting the neurons of at least a second culture for a period of time sufficient with a carrier or vehicle which will not stimulate secretion of SNAPPs into the growth medium; d) removing at least a portion of the growth medium of the cultures of b) and c) and performing an isolation step to purify the SNAPPs from the cultures of b) and c); e) quantifying the amount of SNAPPs isolated in e) from the cultures of b) and c); and f) determining that the test compound is a stimulator of SNAPP secretion when the quantity of SNAPPs isolated from b) are significantly increased when compared with the amount of SNAPPs in c).

In accordance with yet another embodiment, the present invention provides a method for screening for compounds which inhibit secretion of secreted neuronal-activity induced proteasomal peptides (SNAPPs) comprising the steps of: a) providing a plurality of in vitro cultures comprising a plurality of neurons in a growth medium; b) providing one or more test cultures by contacting the neurons of at least a first culture with a test compound and with a known neuronal stimulant for a period of time sufficient to allow secretion of SNAPPs into the growth medium; c) providing a negative control by contacting the neurons of at least a second culture for a period of time sufficient with a carrier or vehicle which will not stimulate secretion of SNAPPs into the growth medium; d) providing a positive control by stimulating the neurons of a third culture for a period of time sufficient with a known neuronal stimulant to allow secretion of SNAPPs into the growth medium; e) removing at least a portion of the growth medium of the cultures of b) to d) and performing an isolation step to purify the SNAPPs from the cultures of b) to d); f) quantifying the amount of SNAPPs isolated in e) from the cultures of b) to d); and g) determining that the test compound is a inhibitor of SNAPP secretion when the quantity of SNAPPs isolated from b) are significantly reduced when compared with the amount of SNAPPs in c) and/or d).

The isolation and quantification of SNAPPs can be performed by various methods in the art. In some embodiments the SNAPPs can be isolated various chromatographic methods, including, for example, UHPLC Hydrophilic Interaction Chromatography (HILIC), normal phase, and/or reverse-phase C18 chromatography. These methods can be combined with ultraviolet-visible (UV-vis) spectrophotometry, and other detection methods, to detect the SNAPPs eluting at various times off the different columns.

In accordance with some embodiments, the sequences of SNAPPs can be identified with many known methods. In an embodiment, advanced mass spectrometric techniques after fractionation using matrix assisted laser desorption/ionization after HPLC (LC-MALDI) or fractionation of an HPLC column directly into an electrospray mass spectrometer (LC/MS-ESI) can be used to identify the specific SNAPPs. Other methods, such as Edman degradation and sequencing can be used.

Considering that many neurodegenerative disorders may result from improperly degraded proteins, we have tested whether the NMP is at all dysregulated in mouse models for neurodegeneration. Interestingly, in accordance with some aspects of the present invention, the inventors found that the NMP is significantly perturbed very early in a disease model of Alzheimer's (FIG. 8).

As such, in accordance with an embodiment, the present invention provides a method for identifying a neuron or population of neurons as having aberrant or dysregulated NMP function comprising: a) providing at least one first in vitro culture comprising a neuron or population of neurons of interest; b) providing at least one second in vitro normal or control cultures comprising a wild type or standard neuron or population of neurons; c) contacting the neurons of the first and second cultured with a stimulant compound for a period of time sufficient to allow secretion of SNAPPs into the growth medium; c) providing a negative control in vitro culture comprising a wild type or standard neuron or population of neurons by contacting the neurons of the negative control for a period of time sufficient with a carrier or vehicle which will not stimulate secretion of SNAPPs into the growth medium; d) removing at least a portion of the growth medium of the cultures of a) to c) and performing an isolation step to purify the SNAPPs from the cultures of a) to c); e) quantifying the amount of SNAPPs isolated in e) from the cultures of a) to c); and f) determining that the first in vitro culture of interest has dysregulated NMP function when the quantity of SNAPPs isolated from a) are significantly increased or decreased when compared with the amount of SNAPPs in b).

In some embodiments, the above methods can be performed using cysteine or methionine amino acids labeled with ³⁵S added to the culture medium prior to performing the methods of the present invention. Other labeled amino acids known in the art can also be used.

For example, the above methods can be used to compare the NMP function of neurons having known neurodegenerative diseases or models for such diseases to normal neuronal function to determine which neurological diseases or conditions are associated with dysregulated or aberrant NMP function.

In accordance with an embodiment, the present invention provides a method for modulating the NMP in neuronal cells in a subject comprising administering to the subject an effective amount of a NMP stimulator or inhibitor to the subject.

In accordance with another embodiment, the present invention provides a method for modulating an NMP associated disease or disorder of neuronal cells in a subject comprising administering to the subject an effective amount of a NMP stimulator or inhibitor to the subject.

Examples of proteasomal stimulators useful in the inventive methods can include, but are not limited to, PA28, PA200, PA700, arginine-rich histone H3), small molecules (oleuropein, betulinic acid—and derivtives), lipid activators (lysophosphatidylinositol, cardiolipin, ceramides), fatty acids (linoleic, oleic, linolenic acids), synthetic peptidyl alcholos (pnitroanilides, nitriles). (Curr Med Chem. 2009; 16(8):931-939).

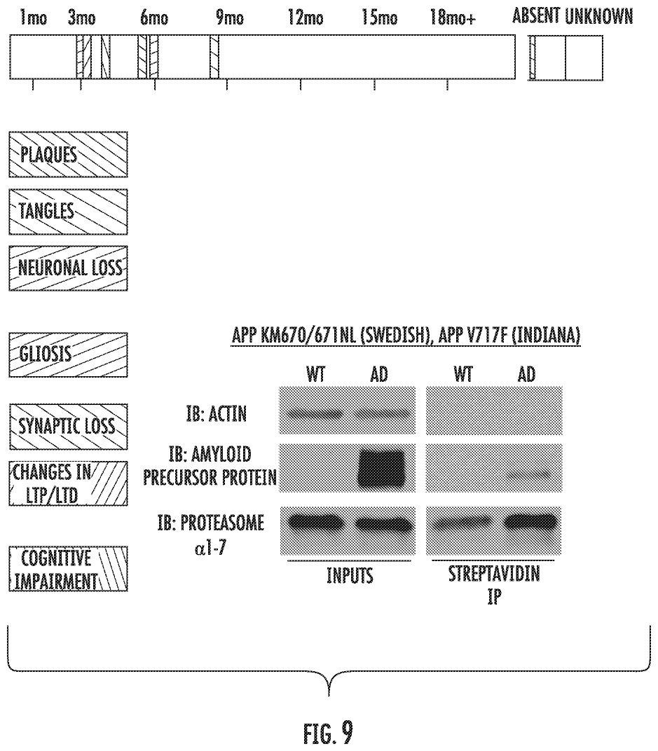

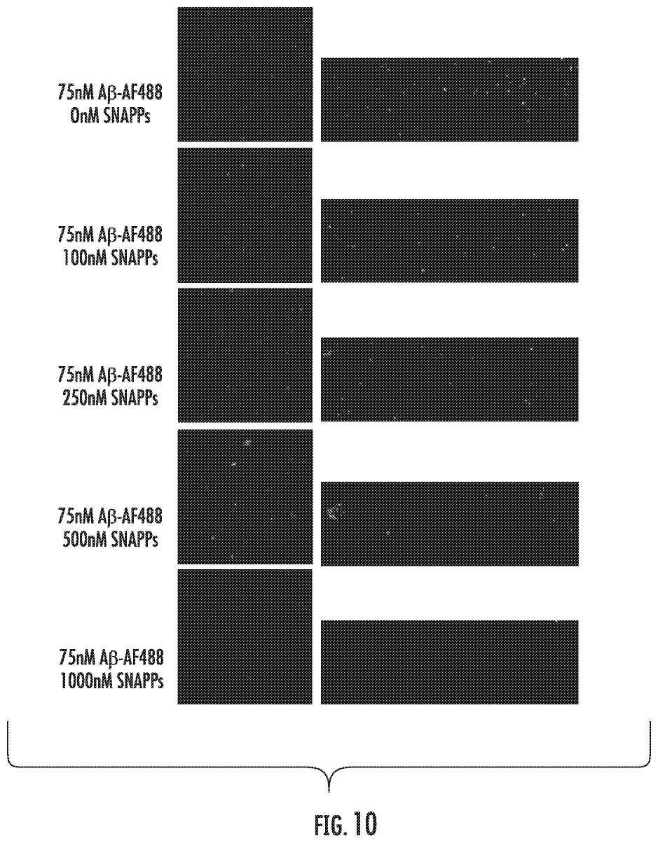

As seen in FIG. 9, it is thought that certain neurological diseases can be caused in whole or in part, by dysregulation of the NMP in the neuronal cells. Certain disorders may in fact, be caused by disruption of NMP function or under expression of NMP proteins, causing a decrease in SNAPP production which may cause under stimulation of neural pathways downstream from the affected cells. Conversely, certain disorders may in fact, be caused by an excess of NMP function or over expression of NMP proteins, causing an increase in SNAPP production which may cause over stimulation of neural pathways downstream from the affected cells, and which may lead to neuronal apoptosis and death. Examples of diseases which may be affected by NMP signaling include, but are not limited to psychiatric disorders, epilepsy, multiple sclerosis, autism, Alzheimer's disease, Parkinson's disease, amyotrophic lateral sclerosis, Huntington's, aging, dementia, enhancement learning and memory and other neurodegenerative diseases.

In accordance with a further embodiment, the present invention provides a method for inhibiting neuronal activity or cognitive function in a subject comprising administering to the subject, an effective amount of NMP inhibitor to the subject.

It will be understood by those of skill in the art that by inhibition of the NMP activity on neurons, either through downregulation of expression of NMP or through direct inhibition with an inhibitory agent, the neurons, when stimulated, will release less SNAPPs into their surrounding environment. This can potentially result in lesser post-synaptic stimulation of surrounding neurons and diminished post-synaptic activity as a result of pre-synaptic stimulation. While not being bound to any particular theory, it is thought that downregulation of NMP expression in neurons, or direct inhibition through the use of inhibitory agents such as NMP inhibitors will have an inhibitory effect on basal neural activity. These effects could be useful in neurological diseases where there is a loss of inhibitory neuronal function. Examples of such diseases include, but are not limited to, epilepsy, encephalopathy, seizures due to other conditions, such as brain tumors, chronic pain, Parkinson's disease, Huntington's disease and other muscle spasm disorders.

In accordance with a yet another embodiment, the present invention provides a method for stimulating or enhancing neuronal activity or cognitive function in a subject comprising administering to the subject, an effective amount of NMP stimulator to the subject.

It will be understood by those of skill in the art that by stimulation of the NMP activity on neurons, either through upregulation of expression of NMP or through direct stimulation with an excitory agent, the neurons, when stimulated, will release increased amounts of SNAPPs into their surrounding environment. This can potentially result in greater post-synaptic stimulation of surrounding neurons and increased post-synaptic activity as a result of pre-synaptic stimulation. While not being bound to any particular theory, it is thought that upregulation of NMP expression in neurons, or direct stimulation through the use of stimulatory/agonist agents such as proteasomal stimulators will have a stimulatory effect on basal neural activity. Moreover, it will be understood by those of skill in the art that modulation of SNAPP release or NMP activity can lead to reversal of neuronal disease states. These effects could be useful in neurological diseases or cognitive conditions where there is a loss of excitatory neuronal function. Examples of uses include, but are not limited to psychiatric disorders, epilepsy, multiple sclerosis, autism, Alzheimer's disease, Parkinson's disease, amyotrophic lateral sclerosis, Huntington's, aging, dementia, enhancement learning and memory and other neurodegenerative diseases.

In some embodiments, the NMP stimulators or inhibitors are combined with a pharmaceutically acceptable carrier as described herein. Moreover, the proteasomal stimulators or inhibitors can be combined with other biologically active agents.

In accordance with an embodiment, the present invention provides a method for stimulating or enhancing neuronal activity or cognitive function in a subject comprising administering to the subject, an effective amount of SNAPPs to the subject.

These effects could be useful in neurological diseases or cognitive conditions where there is a loss of excitatory neuronal function. Examples of uses include, but are not limited to psychiatric disorders, epilepsy, multiple sclerosis, autism, Alzheimer's disease, Parkinson's disease, am otroplaie lateral sclerosis, Huntington's, aging, dementia, enhancement learning and memory and other neurodegenerative diseases.

In some embodiments, the one or more SNAPPs are combined with a pharmaceutically acceptable carrier as described herein. Moreover, the SNAPPs can be combined with other biologically active agents.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released the subject composition, for example, into adjacent tissues or fluids upon administration to a subject.

Examples of active agents that can be used with the inventive SNAPPs, and NMP stimulators or inhibitors, and methods include, but are not limited to autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, and adrenergic agonist sympathomimetics, anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics, skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists, psychotropic agents, such as antidepressants, heterocyclic antidepressants, monoamine oxidase inhibitors selective serotonin re-uptake inhibitors tricyclic antidepressants, antimanics, anti-psychotics, phenothiazine antipsychotics, anxiolytics, sedatives, and hypnotics, barbiturate sedatives and hypnotics, benzodiazepine anxiolytics, sedatives, and hypnotics, and psychostimulants.

In another embodiment, the term “administering” means that at least one or more SNAPPs or NMP stimulators or inhibitors of the present invention are introduced into a subject, preferably a subject receiving treatment for a disease, and the at least one or more SNAPPs or NMP stimulators or inhibitors are allowed to come in contact with the one or more disease related cells or population of cells in vivo.

As used herein, the term “treat,” as well as words stemming therefrom, includes diagnostic and preventative as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

### EXAMPLES

Antibodies.

The following were used according to manufacturer's and/or published suggestions for western blotting and immunocytochemistry: anti-α1-7 proteasome subunit (Enzo), anti-β2 proteasome subunit (Cell Signaling), anti-α5 proteasome subunit (Santa Cruz), anti-β1 proteasome subunit (Santa Cruz), anti-β2 proteasome subunit (Santa Cruz), anti-β2 proteasome subunit (Enzo), anti-β2 proteasome subunit (Novus), anti-β2 proteasome subunit (Santa Cruz), anti-β5 proteasome subunit (Santa Cruz), anti-β5 proteasome subunit (Enzo), anti-Rpt5 proteasome subunit (Enzo), anti-calregulin (Santa Cruz), anti-β-Actin (Abcam), anti-Biotin (Cell Signaling), Streptavidin-AF647 (Invitrogen), anti-Tubulin (Milipore), anti-GluR1 (Cell Signaling), anti-Myc (Abcam), anti-Transferrin (Invitrogen), anti-EphB2 (M. Greenberg)58, anti-NGluR1 (R. Huganir), cleaved Caspase-3 (Cell Signaling), anti-Kv1.3 (NeuroMab), anti-S2 (Milipore), anti-PA200 (Novus), anti-11Sα (Cell Signaling), anti-11Sβ (Cell Signaling). Antibodies obtained from commercial vendors were verified for specificity using western blotting, immunofluorescence, or immunoprecipitation. We prioritize those antibodies with a continued record of use in multiple independent studies (Table A). For proteasome antibodies, many antibodies used recognize a single band or set of bands at the known molecular weight. Genetic validation of these antibodies is impossible as all proteasome subunits are essential and no knockout controls can be obtained.

TABLE A
List of Antibodies Used

| ANTIBODY | COMPANY | HOST | REACT. | CAT. NO. | Clone | Ref. |
|---|---|---|---|---|---|---|
| α1-7 proteasome | Enzo Life | Mouse | H, M, R, Rb | BML- | MCP231 | See online |
| subunit | Sciences | | | PW8195- | | data |
| | | | | 0025 | | sheet^{1,2}. |
| α2 proteasome | Cell | Rabbit | H, M, R, Mk | 2455 | * | See online |
| subunit | Signaling | | | | | data |
| | | | | | | sheet³. |
| α5 proteasome | Santa Cruz | Mouse | H, M, R | sc-271378 | D-9 | See online |
| subunit | | | | | | data |
| | | | | | | sheet. |
| β1 proteasome | Santa Cruz | Rabbit | H, M, R | sc-67345 | FL-241 | See online |
| subunit | | | | | | data |
| | | | | | | sheet. |
| β2 proteasome | Enzo Life | Mouse | H (97% | BML- | MCP168 | See online |
| subunit | Sciences | | identical to | PW8145- | | data |
| | | | mouse) | 0025 | | sheet [symbol] . |
| β2 proteasome | Novus | Mouse | H, M, R | NBP1-92294 | * | See online |
| subunit | Biologicals | | | | | data |
| (Antibody 1) | | | | | | sheet. |
| β2 proteasome | Santa Cruz | Goat | H, M, R | sc-54676 | S-18 | See online |
| subunit | | | | | | data |
| (Antibody 2) | | | | | | sheet. |
| β5 proteasome | Enzo Life | Rabbit | H (100% | BML- | * | See online |
| subunit | Sciences | | identical to | PW8895- | | data |
| (Antibody 1) | | | mouse) | 0100 | | sheet [symbol] . |
| β5 proteasome | Santa Cruz | Goat | H, M, R | sc-55009 | C-19 | See online |
| subunit | | | | | | data |
| (Antibody 2) | | | | | | sheet. |
| Rpt5 | Enzo Life | Mouse | H, M, R, Rb | BML- | TBP1-19 | See online |
| regulatory | Sciences | | | PW8770- | | data |
| subunit | | | | 0025 | | sheet |
| S2 | Milipore | Rabbit | H, M | 539166 | * | See online |
| regulatory | | | | | | data |
| subunit | | | | | | sheet. |
| PA200 | Novus | Rabbit | H, M | NBP2-22236 | * | See online |
| regulatory | | | | | | data |
| subunit | | | | | | sheet. |
| 11 [symbol] α | Cell | Rabbit | H, M, Rb | 9643 | D1C10 | See online |
| regulatory | Signaling | | | | | data |
| subunit | | | | | | sheet. |
| 11 [symbol] β | Cell | Rabbit | H, M, Rb | 2409 | * | See online |
| regulatory | Signaling | | | | | data |
| subunit | | | | | | sheet. |
| Kv1.3 | NeuroMab | Mouse | H, M, R | 75-009 | L23/27 | See online |
| | | | | | | data |
| | | | | | | sheet. |
| Calregulin | Santa Cruz | Goat | H, M, R | sc-7431 | T-19 | See online |
| | | | | | | data |
| | | | | | | sheet. |
| β-Actin | Abcam | Mouse | H, M, Rb . . . | ab8226 | mAbcam | See online |
| | | | | | 8226 | data |
| | | | | | | sheet. |
| Tubulin | EMD | Mouse | H, M, Rb . . . | MAB1637 | TU-20 | See online |
| | Milipore | | | | | data |
| | | | | | | sheet [symbol] . |
| GluR1 | Cell | Rabbit | H, M, R | 13185 | D4N9V | See online |
| | Signaling | | | | | data |
| | | | | | | sheet. |
| Myc | Abcam | Mouse | H, M | ab32 | * | See online |
| | | | | | | data |
| | | | | | | sheet. |
| Transferrin | Invitrogen | Mouse | M, R, C | 136800 | H68.4 | See online |
| | | | | | | data |
| | | | | | | sheet. |
| Caspase-3 | Cell | Rabbit | H, M, R | 9664 | 5A1E | See online |
| | Signaling | | | | | data |
| | | | | | | sheet. |

[symbol]

indicates data missing or illegible when filed

Mice.

All animal procedures were performed under protocols compliant and approved by the Institutional Animal Care and Use Committees of The Johns Hopkins University School of Medicine. No difference was observed in experiments performed distinguishing between sexes. As such, both male and female mice were considered for analyses for this study. For all experiments, we use wild-type C57BL/6 mice (stock number 027 from Charles River Laboratories). These are general-use animals that are used by many laboratories in the field. The specific age of animal used is listed in the experimental procedure sections. For the majority of experiments, mice were euthanized with carbon dioxide-induced anoxia and decapitated as a secondary method of euthanasia. For in vivo experiments, animals were anesthetized with isofluorane and then decapitated.

Perfusion.

P30 WT C57B1/6 Mice were anesthetized with Isoflourane and rapidly perfused with phosphate buffer and 0.5% paraformaldehyde/1.0% glutaraldehyde and brains were thin-sectioned for Immuno-EM analysis.

Immuno-Electron Microscopy and Analysis.

Brain slices from perfused mice and neuronal cultures were fixed and processed for Electron Microscopy. EM Grids were incubated in the primary antibody overnight at 4° C. followed by secondary antibodies for 2 hours at room temperature. All grids were viewed with a Phillips CM 120 TEM operating at 80 Kv and images were captured with an XR 80-8 Megapixel CCD camera by AMT. Neuronal cultures were fixed in 1.5% glutaraldehyde (EM grade, Pella) buffered with 70 mM sodium cacodylate containing 3 mM MgCl2 (356 mOsmols pH 7.2), for 1 hour at room temperature. Thin-sectioned fixed brain slices and neuronal cultures were processed using the following protocol. Following a 30 minute buffer rinse (100 mM cacodylate, 3% sucrose, 3 mM MgCl₂, 316 mOsmols, pH 7.2), samples were post-fixed in 1.5% potassium ferrocyanide reduced 1% osmium tetroxide in 100 mM cacodylate containing 3 mM MgCl₂, for 1 hr in the dark at 4° C. After en-bloc staining with filtered 0.5% uranyl acetate (aq.), neurons were dehydrated through graded series of ethanols and embedded/cured with Eponate 12 (Pella). LR-white procedural staining was used for HEK293 cells as well as neuronal cultures. A metal hole punch was used to remove 5 mm discs from the polymerized plates. Discs were mounted onto epon blanks and trimmed. Sections were cut on a Reichert Ultra cut E with a Diatome diamond knife. 80 nm sections were picked up on formvar coated 200 mesh nickel grids and treated for antigen removal followed by on grid immunolabelling. Grids were floated on 95° C. citrate buffer pH 6.0 in a porcelain staining dish for 25 minutes, and then allowed to cool on the same solution for 20 min. After a brief series of 50 mM TBS rinses, grids were floated on 50 mM NH₄Cl in TBS, blocked with 2% horse serum in TBS (no tween) for 20 minutes. Grids were incubated in primary antibody diluted in blocking solution (1-50 Goat, mouse, rabbit antibody). Grids incubated on blocking solutions served as negative controls. Sections were allowed to come to room temperature (1 hour) on antibody solutions and placed on appropriated blocking solutions for 10 min. After further TBS rinses, grids were floated upon 12 nm Au conjugated donkey anti-goat, 12 nm Au conjugated goat anti-rabbit, 12 nm Au conjugated donkey anti-mouse, or Au conjugated streptavidin (Jackson Immunoresearch) at 1-40 dilutions in TBS for 2 hours at room temperature. Grids were then rinsed in TBS, floated upon 1% glutaraldehyde for 5 min, rinsed again and stained with 2% filtered uranyl acetate. All grids were viewed with a Phillips CM 120 TEM operating at 80 Kv and images were captured with an XR 80-8 Megapixel CCD camera by AMT.

Cell Lines

For primary mouse neuronal cultures, pregnant wild-type C57/B6 mice were obtained from Charles River Laboratories, and sacrificed at E17.5. Whole cortices were dissected, processed into a single cell suspension, and plated as previously described⁵⁸. Primary cell lines isolated in our laboratory from mouse brains are identified by surface markers that are unique to neuronal cells. These approaches have high sensitivity to accurately identify specific cells. Alternatively, for biochemical studies analysis of primary cell lines can be done using western blotting with well-validated antibodies to neuronal specific markers. Human Embryonic Kidney (HEK293) and Neuro-2A neuroblastoma cells were obtained from ATCC and maintained and expanded and frozen down in a series of aliquots. These aliquots are cultured for a limited number of passages (<10). They are regularly tested for any infection. The lab maintains strict guidelines for cell culture and monitoring of cell health in order to minimize biological variability and to prevent cell line cross-contamination during culture. Each cell line is maintained in its own culture medium.

Cell Culture and Transfection.

HEK293 and Neuro2A cells were cultured in DMEM supplemented with 10% fetal bovine serum, 2 mM glutamine (Sigma), and penicillin/streptomycin (100 U/mL and 100 μg/mL, respectively; Sigma). Mouse cortical neurons were prepared from E17.5 C57B1/6 mouse embryos as previously described⁵⁸. Neurons were maintained in Neurobasal Medium (Invitrogen) supplemented with 2% B-27 (Invitrogen), penicillin/streptomycin (100 U/mL and 100 μg/mL, respectively), and 2 mM glutamine. Dissociated neurons were transfected using the Lipofectamine method (Invitrogen) according to the manufacturer's suggestions.

Each cell line is maintained in its own culture medium. Neurons were maintained in Neurobasal Medium (Invitrogen) supplemented with 2% B-27 (Invitrogen), penicillin/streptomycin (100 U/mL and 100 μg/mL, respectively), and 2 mM glutamine. HEK293 cells were cultured in DMEM supplemented with 10% fetal bovine serum, 2 mM glutamine (Sigma), and penicillin/streptomycin (100 U/mL and 100 μg/mL, respectively; Sigma).

For analyzing the expression of immediate-early gene products, unique care was taken to ensure that neurons had reduced activity at baseline as measured by the expression of immediate early genes. After switching 500K neurons/well in 12 well format of cultured cortical neurons into 1 mL Neurobasal/B27 at DIV3, neurons were maintained in that medium, with only one 100 μl media exchange at DIV9. At DIV15, neurons were treated with pharmacological agents as indicated. Great caution was taken to minimize physical perturbation of these cultures so as not to induce any activation of IEG proteins. For example, drugs were resuspended in a small volume of growth media (media in which neurons were growing in) before addition, so cultures did not have to be shaken to treat neurons.

Antibody Feeding and Immunocytochemistry.

Cultured cortical neurons were plated on glass coverslips coated with poly-L lysine overnight. Neurons were allowed to mature to DIV 14 for feeding experiments. DIV 14 cortical neurons were slowly washed twice with cold PBS supplemented with 1 mM CaCl₂ and 2 mM MgCl₂ to slow recycling and internalization. Care was taken not to shear cell bodies from the neuron, and to maintain neuronal morphology. Cold neurons, while alive, were treated with Chicken anti-MAP2 antibodies (1:100), Goat anti-β5 proteasome subunit antibodies (1:50), and Rabbit anti-GluR1 (1:100) in PBS supplemented with 1 mM CaCl2 and 2 mM MgCl₂ for 30 minutes at 4° C. Antibodies were washed off, and neurons were rinsed twice in cold PBS, 1 minute each. Neurons with bound antibodies were fixed in 4% paraformaldehyde/4% sucrose in PBS for 75 seconds, so not to destroy the antibody itself but to maintain neuronal morphology. Samples were visualized using donkey anti-goat AF-488, donkey anti-chicken AF-555, and donkey anti-rabbit AF-647 (1:250 each) in 1×non-permeabilizing GDB (30 mM phosphate buffer pH 7.4 containing 0.2% gelatin, and 0.8 M NaCl) for 1 hour at 25° C. Samples on coverslips were mounted on glass slides using Fluoromount-G (Southern Biotech). Neurons were imaged using a laser scanning Zeiss LSM780 FCS microscope. Images are representative maximal Z projections of multiple optical sections.

Protease Protection Assay.

Cortical neuronal cultures were treated for the indicated times with 1 μg/mL of Proteinase K (NEB) in HBSSM (Hank's Balanced Salt Solution without CaCl₂ or phenol red, supplemented with 1 mM MgCl₂). Excess Proteinase K was quickly washed away three times in HBSSM, and Proteinase K activity was quenched twice for 3 minutes with 10 μM PMSF in HBSSM at 4° C. Neurons were then fractionated into cytosolic and membrane fractions as described above, and samples were prepared for SDS-PAGE and western analysis.

Surface Biotin-Labeling, Cell Lysis, Streptavidin Pulldown, and Western Blots.

Surface biotin-labeling was performed as previously described²⁶. Whole mouse brains, cultured cells or whole animal tissue were obtained where indicated and each sample was labeled using Sulfo-NHS-LC-Biotin (ThermoFisher). Cultured cells were washed in pH 8.0 PBS (Gibco) with 1 mM CaCl2 and 2 mM MgCl₂ (PBSCM) and treated with 1 mg/mL Sulfo-NHS-LC-Biotin dissolved in PBSCM for 20 minutes at 4° C. before the reaction was quenched for 10 minutes in 50 mM glycine in PBSCM. Intact tissue was quickly and manually chopped, following biotinylation for only 10 minutes at 4° C. in 0.5 mg/mL Sulfo-NHS-LC-Biotin prior to quenching the reaction. Whole mouse tissues and cultured neurons were collected and homogenized in RIPA buffer (50 mM Tris pH 8.0, 150 mM NaCl, 1% Triton X-100, 0.5% Sodium Deoxycholate, 0.1% SDS, 5 mM EDTA, complete protease inhibitor cocktail tablet (Roche), 1 mM β-glycerophosphate). Where indicated, the salt concentration in our RIPA lysis buffer was increased up to 300 mM NaCl. Primary, human central nervous system (CNS) tissue, gestational weeks 19-21. were obtained under surgical written consent following protocols approved by the Johns Hopkins Institutional Review Board, based on its designation as biological waste. Tissue was mechanically chopped at 4° C., and immediately processed for surface biotinylatioti. For streptavidin pulldown experiments, lysed cells were incubated with high-capacity streptavidin agarose beads (ThermoFisher) overnight and then washed thrice with RIPA buffer before elution in SDS sample buffer. Western blots were performed using conventional approaches. Gels were run either on 4-15% SDS-PAGE gradient gels (Bio-Rad) or on 10% gels made in the laboratory. Proteins were transferred to nitrocellulose membranes at 100V for 1.5 hours in 20% methanol containing transfer buffer. All antibodies were made up in 5% BSA in 0.1% TBST. Western blots were incubated with appropriate secondary antibodies coupled to Horseradish Peroxidase, extensively washed, and incubated with ECL. Images were exposed on film, and were scanned in and quantified using ImageJ by standard densitometry analysis.

Cellular Fractionation and Integral Membrane Determination

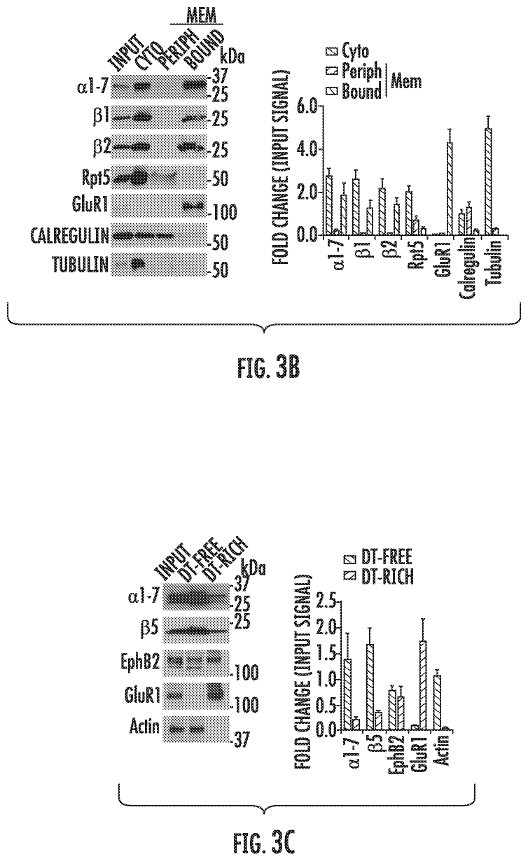

For cellular fractionation experiments to determine the membrane attachment of the proteasome, cultured neurons were lysed in either a sucrose buffer (0.32 M sucrose, 5 mM HEPES, 0.1 mM EDTA, 0.25 mM DTT) or hypotonic lysis buffer (5 mM HEPES, 2 mM ATP, 1 mM MgCl₂) collected. Nuclei were pelleted at 800 RPM for 5 minutes, and the supernatant containing membranes was pelleted at 55000 RPM for 1 hour. Pelleted membranes were washed twice by homogenizing in lysis buffer and re-pelleted. Following two washes, membranes were processed for appropriate application. Supernatants containing the cytosolic extracts were concentrated down to the same volume that membranes were eventually resuspended in. Membrane association was determined by classic methods of sodium carbonate extraction. Briefly, purified neuronal membranes were resuspended in 50 mM sodium carbonate, pH 11 and incubated for 10 minutes at 4° C. to strip away membrane-associated proteins. Membranes, along with tightly-associated membrane proteins, were pelleted at 55000 RPM for 1 hour. Incubating membranes with sodium carbonate at high pH is thought to strip peripherally-associated proteins from the membranes, leaving only tightly-associated and integral membrane proteins bound to the membranes. Samples were subsequently prepared for SDS-PAGE analysis. For Digitonin fractionation, samples were lysed in sucrose buffer. Once the supernatant (cytosolic fraction) was set aside, the pellet was washed 2× with sucrose buffer, and then resuspended in sucrose buffer with indicated concentrations of digitonin. Following a 30 minute incubation in the buffer, samples were spun down at 55000 RPM for 1 hour. This was repeated for all indicated concentrations of detergent. For FIG. 3A, based on our fractionation protocol, we calculated that the input was about 60% cytosol and 40% membrane. We only collected the non-nuclei, non-mitochondria membrane (i.e. 20% of remaining membranes). For our westerns in FIG. 3A we used 10 μl of input and ˜3×-purified cytosol and ˜5×-purified membrane. Combining the data from the cytosol and membrane fractions and considering error in our experimental approach proteasome signal from our input is likely coming from both the cytosol and a larger fraction from the membrane preparations. Because our input includes all the cellular material and the fractionation removes the nuclei and mitochondria we believe, if any, a very small amount of proteasome signal in our input can account for that which is coming from these organelles.

TX-114 Phase Extraction.

Protocol was adapted from³³. Briefly, primary neuronal cultures were treated with 1% precondensed TX-114. Samples were dounce homogenized, spun at 4° C., and incubated at 30° C. Samples were centrifuged for 3 minutes at room temperature. Supernatant was retained as the TX114-free fraction and resulting pellet was kept as the TX114-rich fraction. This approach relies upon a temperature-dependent shift of the critical micellar concentration of TX-114, and provides an approximate determination of the hydrophobicity of proteins.

Concanavalin-A Plasma Membrane Isolation.

Protocol was adapted from³¹. Briefly, 0.25 mg biotinylated Concanavalin-A (ConA) was first coupled to 1 mL of streptavidin-coated agarose beads. Nuclei were pelleted from hypotonically lysed DIV 16 cultured cortical neurons, as described above, and the supernatant containing plasma membranes and cytosol were applied to 150 ul of ConA beads. After thorough washing in lysis buffer containing 0.025% Nonidet-P40, samples were prepared for SDS-PAGE and western analysis.

DNA Constructs.

The full-length mouse tagged GPM6A, tagged GPM6B, tagged β5 constructs were acquired from Origene. All vectors obtained from commercial sources are verified and tested for the appropriate expression of the inserts using primary antibodies or epitope-tag antibodies against the expressed proteins. While we keep stocks of each validated plasmid, we periodically sequence these plasmids to confirm their authenticity. All plasmids used in this study are amplified and purified using standard kits from commercial vendors.

shRNA Knockdown.

Four unique shRNA constructs were obtained each against GPM6A, GPM6B, and PLP from Origene. These were validated HuSH 29mer shRNA constructs expressing GFP. Each construct was transfected into neurons using previously described and standard protocols. Each construct was transfected at 100 ng and 500 ng/well. In addition, the constructs were co-transfected in combination to knockdown either two, or all three genes.

Human Subjects.

Fetal brain tissue was obtained at Johns Hopkins University. Primary cultures of fetal cortical tissues were prepared. The use of fetal brain tissue was approved by the Johns Hopkins University institutional review board (IRB). Informed consent was obtained from all subjects. The authors did not have access to any identifying personal information.

Co-Immunoprecipitations.

Transfected HEK293 cells were collected and homogenized in IP Buffer (1% NP-40, 2mM MgCl₂, 300 mM NaCl, 2 mM CaCl₂, 50 mM HEPES, 10% Glycerol) buffer. For immunoprecipitations, lysates were incubated with FLAG-M2 agarose beads (Sigma-Aldrich). Precipitated samples were washed and prepared for SDS-PAGE and immunoblot analysis.

Proteasome Purification and Assessment of Catalytic Activity.

For proteasome purification, cells were treated and then immediately put on ice before purifications were performed as previously described⁴⁵. Briefly, proteasomes were purified out of neuronal cytosol and detergent-extracted neuronal plasma membranes using the 20S proteasome purification kit (Enzo Life Sciences) or the 26S proteasome purification kit (UBPBio). The first method relies on immunoprecipitating proteasomes using proteasome β2 or β5 subunit antibodies covalently coupled to agarose beads (20S purification matrix). It is important to note that this purification scheme can purify any 20S-containing proteasome complex. As an alternative method, we used a previously described affinity purification that utilizes GST-Ubl binding to the 19S cap and subsequent pulldown on Glutathione-coupled sepharose (26S purification matrix). This method enriches for proteasomes that are capped by the 19S complex. For western blots, samples were denatured at 65° C. for 5 minutes in SDS sample buffer, resolved by SDS PAGE, transferred to nitrocellulose, and immunoblotted. For catalytic activity assays, ⅙th of the bead volume following proteasome purification was resuspended in activity assay buffer (20 mM Tris-HCl, pH8.0, 5 mM ATP, 5 mM MgCl₂, 1 mM DTT). 26S Proteasomal activity was assessed by the addition of 10 μM of SUC-LLVY-AMC (Enzo Life Sciences). The contribution of 20S proteasomal activity was assessed by the comparison of 26S proteasome activity to that of total proteasome activity (26S+20S), measured by the activity of samples containing SDS at a final concentration of 0.05%.

Cell Culture Radiolabeling

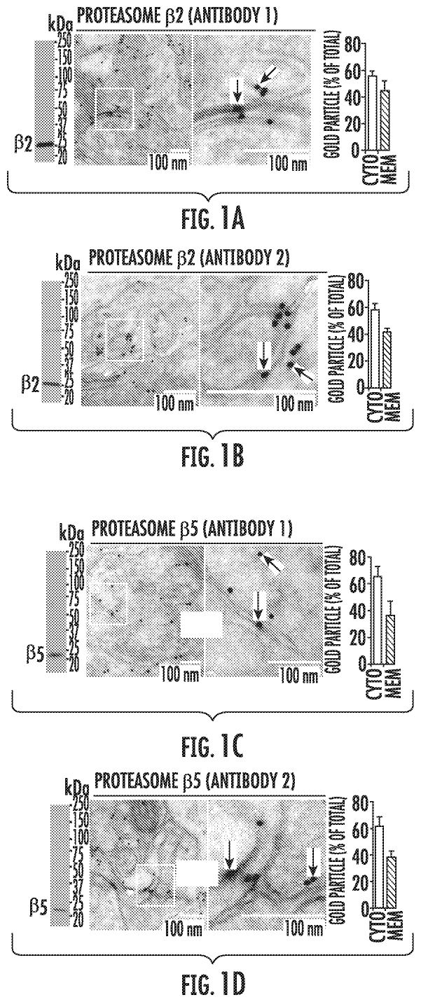

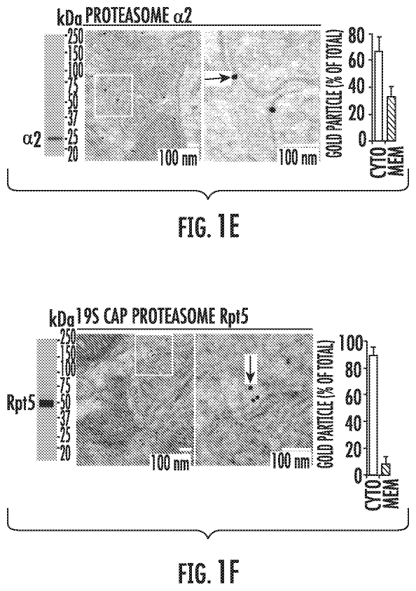

Cortical neurons were cultured for 12 days in vitro. Radioactive labeling was done in Neurobasal growth media with B-27 supplement and without methionine or cysteine (Life Technologies, special order). ³⁵S methionine/cysteine (EasyTag PerkinElmer) was incorporated during indicated times at 55 mCi in the met/cys free growth medium. Where indicated, MG-132 (25 μM, Cell Signaling) and ATPγS (1 mM, Sigma) was added during the radioactive labeling window. For all labeling experiments, normal growth media on neurons was switched into labeling media supplemented with radioactive label for 10 minutes. Lysates were prepared in RIPA buffer (50 mM Tris pH 8.0, 150 mM NaCl, 1% Triton X-100, 0.5% Sodium Deoxycholate, 0.1% SDS, 5 mM EDTA, complete protease inhibitor cocktail tablet (Roche), 1 mM sodium orthovanadate, 1 mM β-glycerophosphate). SDS sample buffer was added and samples were boiled for 5 minutes prior to loading onto SDS-PAGE gels. Autoradiographs were done by loading samples onto large SDS-PAGE gels, coomassie stained to verify equal loading, and then gels were dried down on a large gel drier onto Whatman filter paper. Dried gels were exposed to phosphorimager screens and scanned with a Typhoon FLA5500 imager. A variety of other manipulations and pharmacological agents were used during the pulse-chase protocol as indicated in supplementary FIG. 1. Synaptic activity was blocked by the addition of Tetrodotoxin (1 μM, Tocris), CNQX(1 μM, Tocris), and AP5 (1 μM, Tocris). Alternative stimuli to KCl depolarization included previously reported Glutamate (100 μM), and chemical LTP (125 mM NaCl, 2.5 mM KCl, 2 mM CaCl2, 5 mM Hepes, 33 mM Glucose , 0.2 mM Glycine, 0.02 mM Bicuculline, and 0.003 mM Strychnine) protocols. Neurons were treated with ACSF, chemical LTP buffer, or glutamate for 10 minutes during radiolabeling. 5% FES was added for 30 minutes prior to radiolabeling and during radiolabeling. Media exchange was done by simply replacing growth media with fresh Neurobasal/B27 to account for the stress of replacing media.

Peptide (SNAPP) Collection and Quantification.

Following incorporation of radioactive ³⁵S methionine/cysteine, neurons were rapidly washed in PBS and fresh Neurobasal media without phenol red and with 2×B-27 supplement was added. At the two-minute time point, all of the media was collected and then spun through a 10 kDa Amicon filter (Millipore) and the flow through was then spun through a 3 kDa Amicon filter (Millipore). The flow-through from this sequential filtering was then dialyzed using dialysis tubing with a 100-500 Da cutoff (Spectrum Labs) into either 1×PBS (Gibco) or 20 mM Ammonium Bicarbonate (Sigma). Following four days of dialysis, samples were lyophilized and resuspended in MilliQ water for downstream calcium imaging. Quantification of peptides was done by counting the amount of radioactivity in each sample by liquid scintillation (Wallac 1410). Proteinase K control experiments were done by treating the media with 100 μg/mL proteinase K overnight in 2 M Urea and 10 mM BME, prior to re-dialyzing the proteolyzed media into 2 M Urea for two days, and then gradually reducing the Urea concentration down into NaCl and then into Ammonium Bicarbonate. Resuspended peptides were quantified prior to applications using LavaPep Fluorescent Peptide Quantification Kit (LP022010, Gel Company).

Biotin-Epoxomicin.

Biotin-epoxomicin is de-novo synthesized and purchased from Leiden University Institute of Chemistry. They are fully equipped with synthetic capabilities in organic chemistry. Mass spectrometry and NMR verify all batches produced by his lab for quality and purity. All batches used have had >99% purity. To further minimize batch variation, we test all batches in biological experiments (dose-titration for peptide release, NMP inhibition and cell viability responses).

Biotin-epoxomicin was added to neuronal cultures at 25 μM immediately after labeling. Following peptide release assays, treated cells were lysed in a sucrose homogenization buffer (0.32 M sucrose, 5 mM HEPES, 0.1 mM EDTA, 0.25 mM DTT). Membranes were separated from the cytosol by high-speed centrifugation at 55,000 RPM for 1 hour. Fractions were solubilized in SDS sample buffer prior to loading on SDS-PAGE gels for western analysis. EM processing was done after 5 minutes of treatment with Biotin-Epoxomicin.

Calcium Imaging

Calcium imaging was performed as previously described⁵⁹. Briefly, for the Biotin-Epoxomicin experiments, cultured embryonic cortical neurons were transfected with 1 μg of a mammalian expression construct encoding GCaMP3 at DIV10 and imaged at DIV 12-14. Bicuculline treatment was administered as a 1 μM stimulation in calcium imaging buffer in a perfusion setup. Once the bicuculline stimulation was washed out, biotin-epoxomicin (25 μM) was co-administered with 1 μM Bicuculline in calcium imaging buffer. Each treatment was monitored for three minutes prior to washout. Coverslips were not imaged twice due to Biotin-Epoxomicin being a covalent inhibitor. Cells were ensured to be healthy at the end of the imaging process by stimulating with 55 mM KCl and washing out and assessing for a proper calcium signal. Quantification was done by picking multiple regions of interests in primary and secondary dendrites across multiple coverslips over different imaging days. Data was analyzed using the Time Series Analyzer V3.0 ImageJ plugin and the ROI manager. Data were pooled for all the ROIs to generate a single N value. Brains from P0-P3 mouse pups (Cre-GCaMP3; Nestin-Cre ER) were dissected and plated in Neurobasal-A with B-27 supplement for two weeks. At DIV7, 4-hydroxytamoxifen (4-HT, concentration) was added to induce GCaMP expression. Neurons were imaged in a calcium-imaging buffer (130 mM NaCl, 3 mM KCl, 2.5 mM CaCl₂, 0.6 mM MgCl₂, 10 mM Hepes, 10 mM glucose, 1.2 mM NaHCO₃ pH 7.45). Peptides (SNAPPs) were collected, filtered, and dialyzed and then lyophilized prior to resuspension in 1 mL of MilliQ water and addition onto GCaMP-encoding neurons. 5 μl of resuspended peptides were sufficient to induce the described calcium-induced effects. Peptides treated with Proteinase K were spun through a 10 kDa MW cutoff filter prior to addition onto neurons in order to remove Proteinase K. Pharmacological inhibitors were perfused in at the indicated times at the following concentrations: BAPTA (2 μM), Thapsigargin (5 μM), Tetrodotoxin (1 μM), Nifedipine (1 μM), APV (2 μM).

Mass Spectrometry

Mass spectrometry for proteasomes isolated from cytosolic and membrane fractions was performed at MS Bioworks, LLC. Otherwise, the fractionated peptides were analyzed on an Orbitrap Fusion Lumos Tribrid Mass Spectrometer coupled with the UltiMate™ RSLCnano nano-flow liquid chromatography system (Thermo Fisher Scientific). The peptides from each fraction were reconstituted in 0.1% formic acid and loaded on a Acclaim PepMap100 Nano-Trap Column (100 μm×2 cm, Thermo Fisher Scientific) packed with 5 μm C18 particles at a flow rate of 5 μl per minute. Peptides were resolved at 250-nl/min flow rate using a linear gradient of 10% to 35% solvent B (0.1% formic acid in 95% acetonitrile) over 95 minutes on an EASY-Spray column (50 cm×75 μm ID, Thermo Fisher Scientific) packed with 2 μm C18 particles, which was fitted with an EASY-Spray ion source that was operated at a voltage of 2.0 kV.

Mass spectrometry analysis was carried out in a data-dependent manner with a full scan in the mass-to-charge ratio (m/z) range of 350 to 1550 in the “Top Speed” setting, three seconds per cycle. MS1 and MS2 were acquired for the precursor ion detection and peptide fragmentation ion detection, respectively. MS1 scans were measured at a resolution of 120,000 at an m/z of 200. MS2 scan were acquired by fragmenting precursor ions using the higher-energy collisional dissociation (HCD) method and detected at a mass resolution of 50,000, at an m/z of 200. Automatic gain control for MS1 was set to one million ions and for MS2 was set to 0.05 million ions. A maximum ion injection time was set to 50 ms for MS1 and 100 ms for MS2. MS1 was acquired in profile mode and MS2 was acquired in centroid mode. Higher-energy collisional dissociation was set to 35 for MS2. Dynamic exclusion was set to 30 seconds, and singly-charged ions were rejected. Internal calibration was carried out using the lock mass option (m/z 445.1200025) from ambient air.

Statistics

No statistical methods were used to predetermine sample size. The experiments were not randomized. All statistical analyses were performed using Origin Prism and Graphpad software, accounting for appropriate distribution and variance to ensure proper statistical parameters were applied. Experimental sample sizes were chosen according to norms within the field. The observed magnitude of differences, together with the low replicate variance, permits high power of analysis based on the sample size chosen. For quantification of proteasomal localization by EM analysis, images were acquired by an independent assistant in the Johns Hopkins imaging core not involved in the experimentation and counts were then objectively tallied by a second assistant without knowledge of the experimental groups. Statistical methods used are described in figure legends for the respective EM experiments. For remaining experiments investigators were not blinded to allocation during experiments and outcome assessment.

Statistical analysis using Student's t tests, 1-way ANOVAs and the appropriate post hoc tests were performed as described in each figure legend. P values≤0.05 were considered significant. Notable exceptions to this are in the mass spectrometry data.

Antibodies. The following were used according to manufacturer's and/or published suggestions for immunoblotting: anti-β-Actin (Abcam), anti-Biotin (Cell Signaling), Streptavidin-AF647 (Invitrogen), anti-Arc (Gift from P. Worley, Johns Hopkins, verified against knockout), anti-Fos (Cell Signaling), anti-Npas4 (Gift from Y. Lin, MIT, verified against knockout), anti-PSD-95 (Pierce), anti-Ube3A (Sigma, verified against knockout), anti-Ubiquitin (FK2, Enzo), anti-S6 ribosomal subunit (Cell Signaling), standard secondary antibodies were purchased. We attempted to use antibodies that were verified by knockout controls in either our study, or by other groups. We only used antibodies that provided a signal at the appropriate molecular weight, and where minimal nonspecific bands were observed.

Immunoblot Analysis.

Immunoblots were performed using conventional approaches. Tris/Glycine gels were run on either 10% or 12% gels made in the laboratory. Proteins were transferred to nitrocellulose membranes at 100V for 2 hours in 20% methanol containing transfer buffer. All antibodies were made up in 5% BSA in 0.1% TBST, except for Arc antibody which was made up in 5% Milk in 0.1% TBST. Immunoblots were incubated with appropriate secondary antibodies coupled to Horseradish Peroxidase, extensively washed, and incubated with ECL. Blots were exposed on film, and were scanned in and quantified using ImageJ by standard densitometry analysis.

Ribosome Pelleting

Ribosome-nascent chain complexes were isolated according to well established protocols (Brandman et al., 2012; Duttler et al., 2013). Following various treatments and radiolabelling, neurons were lysed in a buffer containing either 100 ug/mL Cycloheximide or Puromycin (25 mM HEPES pH7.5, 10 mM MgCl2, 20 mM KCl, 50 mM NaCl, 2 mM ATP, 10u SuperASE-In, 20 μM MG-132, 1.5% Triton X-100, protease inhibitors). Lysates were cleared by centrifugation at 10,000 RPM for 10 minutes, and the supernatant was layered onto a 1M sucrose cushion. Ribosome-nascent chain complexes or empty ribosomes (following puromycin treatment) were pelleted via centrifugation at 70,000 RPM in a Ti 70.3 rotor. Supernatants were discarded and ribosomal pellets were washed three times with lysis buffer. 1/10 of the ribosomes were counted by liquid scintillation and the remainder was prepared in SDS loading buffer.

2 Dimensional Gels for Nascent Chain Analysis.

2-dimensional gels to analyze the ribosome-nascent chain complex were performed as previously described (Ito et al., 2011). Briefly, following 30 seconds of radiolabel incorporation at room temperature, neurons were lysed in buffers containing either Cycloheximide or Puromycin. Following lysis, RNCs were isolated as described above. Isolated RNC complexes were resuspended in SDS loading buffer, and then loaded onto neutral pH SDS-PAGE gels to minimize in-gel tRNA hydrolysis. Each samples was run with a few microliters of prestained ladder to delineate the lanes. After running in a single dimension, lanes were cut out of the gel and then incubated with 1N NaOH at 80° C. to degraded any RNA in the sample. This treatment hydrolyzes the ester bond linking the tRNA to its nascent polypeptide, generating a population of radiolabeled proteins whose mass is reduced by the weight of the tRNA (˜25 kDa). Following RNA hydrolysis, samples were run in a second dimension, and then transferred onto nitrocellulose membranes. After exposure for autoradiography, membranes were blocked in BSA and immunoblotted using anti-ubiquitin antibodies.

Protein Extraction, Digestion, and Labeling.

After indicated treatments, the cells were lysed by adding in 6 M urea and 2 M thiourea buffer with protease inhibitor cocktail. The lysates were sonicated with 35% amplitude for 1 min. Protein lysates were centrifuged at 16,000 g at 4° C. to exclude cell debris (pelleting at the bottom), and protein concentration was estimated using a SDS-PAGE method. Briefly, protein lysate was loaded with BSA standard ranging from 0.33 μg to 9 μg on a 3-12% NuPAGE gradient gel and separated for about 0.5 cm. The gel was stained with Colloidal Coomassie G-250 followed by destaining with water. The band intensities were measured by ImageJ software. A total of 200 μg of each sample was reduced with 10 mM dithiothreitol at room temperature for one hour and alkylated with 30 mM iodoacetamide for 20 minutes in the dark. The protein samples were digested using endoproteinase LysC (1:100) at 37° C. for 3 hours followed by sequencing-grade trypsin (1:50) at 37° C. overnight. After the digestion, the peptide samples were subjected to desalting and labeling with 10-plex TMT reagents according to the manufacturer's instructions (Thermo Fisher Scientific) and the 9/10 channels (126, 127N, 127C, 128N, 128C, 129N, 129C, 130N, 130C) were used for labeling. The labeling reaction was performed for one hour at room temperature, followed by quenching with 100 mM Tris-HCl (pH 8.0). The digested and labeled peptides from all 10 channels were pooled.

The peptides were fractionated by basic pH reversed-phase liquid chromatography (bRPLC) into 96 fractions, followed by concatenation into 24 fractions by combining every 24^{th} fractions. Briefly, Agilent 1260 offline LC system was used for bRPLC fractionation, which includes a binary pump, VWD detector, an autosampler, and an automatic fraction collector. In brief, lyophilized samples were reconstituted in solvent A (10 mM triethylammonium bicarbonate, pH 8.5) and loaded onto XBridge C₁₈, 5 μm 250×4.6 mm column (Waters, Milford, Mass.). Peptides were resolved using a gradient of 3 to 50% solvent B (10 mM triethylammonium bicarbonate in acetonitrile, pH 8.5) at a flow rate of 1 ml per min over 50 min collecting 96 fractions. Subsequently, the fractions were concatenated into 24 fractions followed by vacuum drying using SpeedVac. The dried peptides were suspended in 0.1% formic acid.

Data Analysis.

Proteome Discoverer (v 2.1; Thermo Scientific) suite was used for quantitation and identification. During the preprocessing of MS/MS spectra, the top 10 peaks in each window of 100 m/z were selected for database search. The tandem mass spectrometry data were then searched using SEQUEST algorithms against mouse RefSeq protein database (version 84) with common contaminant proteins. The search parameters used were as follows: a) trypsin as a proteolytic enzyme (with up to two missed cleavages); b) peptide mass error tolerance of 10 ppm; c) fragment mass error tolerance of 0.02 Da; and d) carbamidomethylation of cysteine (+57.02146 Da) and TMT tags (+229.162932 Da) on lysine residues and peptide N-termini as a fixed modification and oxidation of methionine (+15.99492 Da) as a variable modification. The minimum peptide length was set to 6 amino acids. Peptides and proteins were filtered at a 1% false-discovery rate (FDR) at the PSM level using percolator node and at the protein level using protein FDR validator node, respectively.

The protein quantification was performed with following parameters and methods. The most confident centroid option was used for the integration mode while the reporter ion tolerance was set to 20 ppm. The MS order was set to MS2 and the activation type was set to HCD. Unique and razor peptides both were used for peptide quantification while protein groups were considered for peptide uniqueness. Reporter ion abundance was computed based on signal-to-noise ratio and the missing intensity values were replaced with the minimum value. The quantification value corrections for isobaric tags and data normalization were disabled while the co-isolation threshold was set to 50%. The highest signal-to-noise ratio value from PSMs for a peptide was used to generate a peptide level abundance followed by averaging peptide level signal-to-noise ratio values for a protein to generate a protein level abundance.

Protein grouping was performed with strict parsimony principle to generate the final protein groups. All proteins sharing the same set or subset of identified peptides were grouped while protein groups with no unique peptides were filtered out. The Proteome Discoverer iterated through all spectra and selected PSM with the highest number of unambiguous and unique peptides.

TMT Differential Expression

The list of quantified proteins exported from Proteome Discoverer 2.1 was utilized as the input for our differential expression analysis. The raw values were organized in a matrix where each column represented a sample and each row a protein. To normalize the raw expression values, we began by loge transforming the matrix with a +1 for computation. Then we median polished the log-transformed values by subtracting the row median from each row, followed by the subtraction of the column median from each column. The resulting normalized expression values for each sample appeared normally distributed and was comparable across samples.

For the detection of differential regulation, we followed the recommendation outline in (Kammers et al., 2015). An empirical Bayes method was employed on the normalized matrix to detect differences between the 3 samples of the biotin-epoxomicin treated group compared to the 6 samples of the control and cycloheximide groups. The empirical Bayes method shrinks individual protein's sample variance towards a pooled estimate, and creates a more stable and powerful inference in differential protein abundance detection.

The output of the differential abundance analysis detected 1340 and 408 proteins to be differentially abundant at the 0.05 and 0.01 level respectively. However, due to the large number of proteins tested, we were more interested in q-values that adjust for multiple comparisons. Using a cutoff of q<0.1, which corresponds to a false discovery rate of 10%, we detect 190 proteins to be differentially abundant in the 2 groups that we defined. Of those 190 proteins, 122 were up-regulated.

For the selection of the colors in the heatmap, we carried out feature-scaling of the normalized expression values on a gene-by-gene basis. For each gene, this process assigns the largest expression a value of 1, and the smallest expression a value of 0. The remaining values are scaled between 0 and 1 based on where they are relative to the largest and smallest expression values. For instance, a feature-scaled value of 0.5 represents an expression level that is halfway between the lowest expression and the highest expression observed for a gene. In other words, this sample's expression is 50% of the maximum fold change away from the lowest and the highest expression values at this gene.

Markov Chains to Model Radioisotope Release

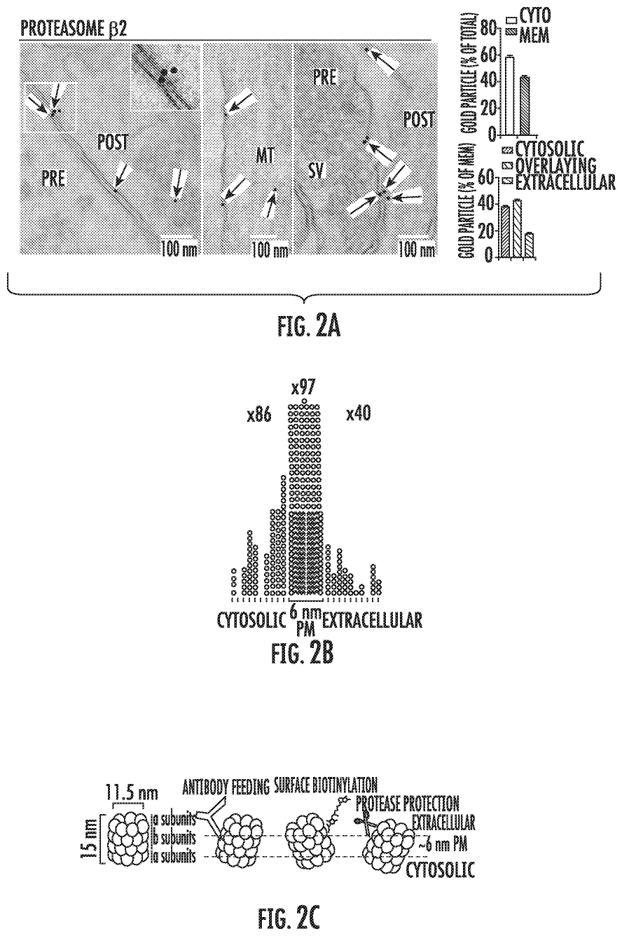

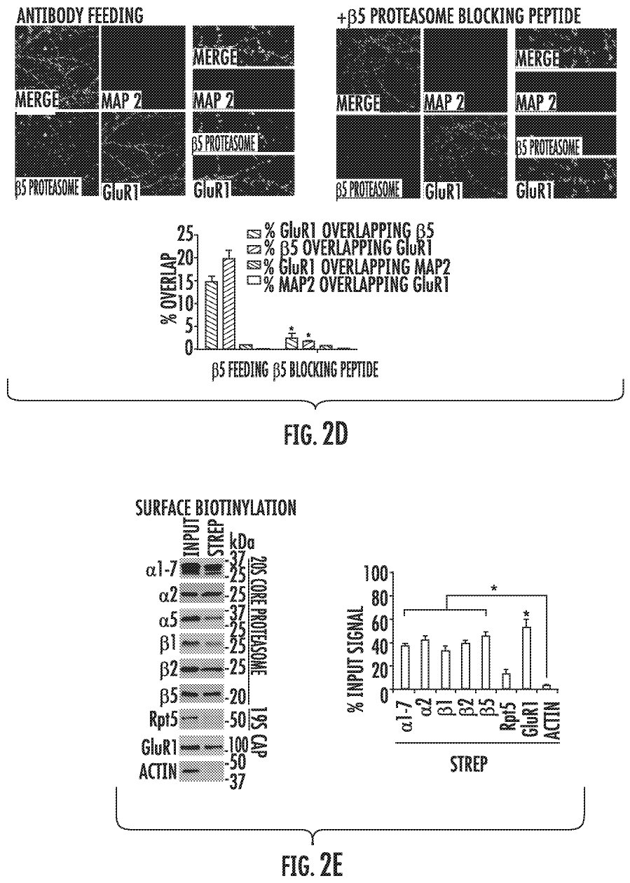

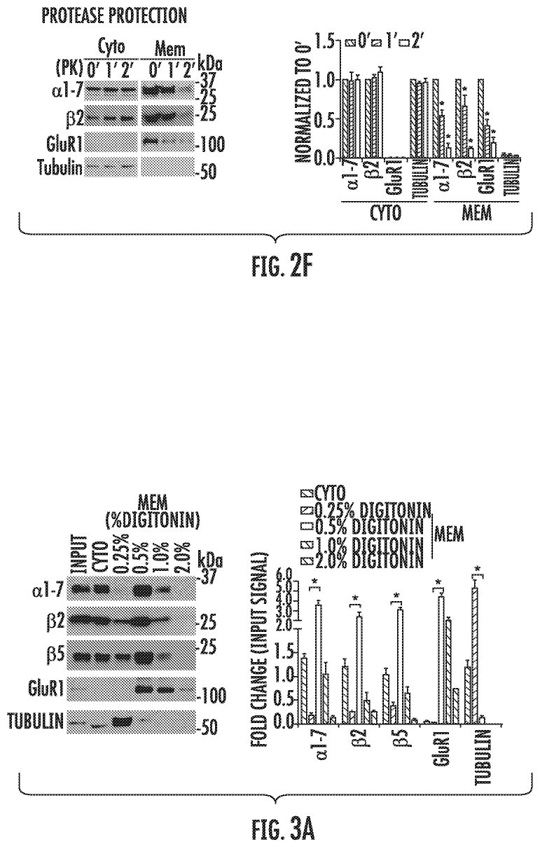

To model the radioisotope release curves that were experimentally observed, we employed Markov chain simulations. A given Markov chain simulates the location of a single radioisotope in 1-second increments, starting at the moment of washout until 1800 seconds (30 minutes) after. The transition process and probabilities between states is given in FIG. 2E. Each radioisotope is assumed to begin as a free isotope within the cell.

A free isotope has at each second interval a pBackground chance of diffusing across the cell membrane to become a free isotope extracellularly. In that same second interval, that isotope also has a pLoading chance of coming in contact with a ribosome and becoming a part of a nascent polypeptide. This leaves that for each interval, a free isotope has a 1-pLoading-pBackground chance of remaining as a free isotope.

Once a radioisotope has progressed to the state of a nascent polypeptide, it has some probability pCTD of being released co-translationally. If entering that release path, the time it takes for the release to be realized extracellularly requires a time that is distributed N(8, sd=2)/2.5. The N(8, sd=2) represents that on average cleave sites are every 8 or so amino acids, while 2.5 is the well-established rate which degradation occurs. If not entering this pathway, the nascent chain becomes a folding intermediate. The time required for this is dependent upon the length of the protein that this isotope is being incorporated into, the location at which it is being incorporated, and the rate of translation. To determine the length of the protein, we sampled a protein at random from the list of detected intracellular proteins under full protein degradation inhibited conditions. The probability of sampling each protein is proportional to their relative abundance. Once the protein has been selected, we simulated the point of incorporation of the radioisotope to be uniform along the length of the full protein. The time to progress from a nascent polypeptide to the folding intermediate is determined as the (# of AA in the protein after the incorporation point/5), with 5AA/s being the established rate translation.

Upon becoming a folding intermediate, the radioisotope has a chance pFID of being degraded and released extracellularly. If entering this degradation path, the time before the radioisotope is realized extracellularly is calculated as the # AA in the protein before the incorporation point (recorded from the previous step) divided by the well-established rate of degradation of 2.5 AA/second. If at this point, the radioisotope does not enter the degradation path, it will initiate the process towards a folded protein.

The time it takes for a folding intermediate to become a folded protein is based upon the power law (Lane and Pande, 2013)and is calculated as a random variable following exp(5*log(#AA)−27.7+Norm(0, sd=3)). This corresponds to a folding time of approximately 30 seconds for a 50 kDa protein. Once the protein is folded, it has a probability pFD of entering degradation in any 1-second interval. If it does enter the pathway, we assume the time it takes for the isotope to be released extracellularly is determined mostly by the unfolding time, which we assumed conservatively to be equal to the folding time distribution. Otherwise, the protein remains folded with a probability of 1-pFD. We chose a pFD of 1e-5 for our model because it corresponded to a conservative representation that the half-life of a folded protein existing in a folded state is approximately 20 hours.

Monte Carlo Inference for Model Parameters

With this formulation of the Markov chain, there remains 4 variables that are not based upon previously established results: pLoading, pBackground, pCTD, and pFID. We employed Monte Carlo simulations in a 2-stage process to optimize those parameters to most closely mirror the experimental observed release curves. Experimental release curves were estimated as follows. For each experimental condition, we have observations of released radioisotope at times 0, 60, 120, 300, 600, and 1800 seconds after washout. The value of each time point was divided by the total amount of radioisotope within the cell at 0 seconds after washout to rescale the observations as a proportion. For any point in time between the 5 observed time points, the released proportion was assumed to follow a linear relationship.

We first exploited the assumption that the dominant isotope release pathway should be diffusion (between 0-600 seconds) in an experimental condition where all degradation of proteins is inhibited. We inferred the optimal values of pLoading and pBackground by exploring the parameter space of all pairwise combinations of pLoading between 0.0035 and 0.0075 in 0.0001 increments and pBackground between 0.00001 and 0.0004 in 0.00001 increments. For each of combination of pLoading and pBackground we used Monte Carlo simulations of 2500 Markov chains, each one starting as a free isotope and having transition probabilities given by the pairwise combination of pLoading and pBackground. The proportion of the 2500 initial radioisotopes that is released extracellularly at each second in time was recorded as the simulated release curve. The simulated release curves were compared to the experimental release curve when all protein degradation was inhibited to determine the optimal combination of pLoading and pBackground. The penalty measure is the sum of the squared distance between observed and simulated at each time point between 1 and 600 seconds. We chose not to evaluate the curves beyond 600 seconds because it appeared reasonable that diffusion was the dominant form of isotope release prior to 600 seconds. For the time range between 600-1800 seconds, other release mechanisms like autophagy might confound our efforts. This process revealed pLoading and pBackground to be optimized at 0.0056 and 0.00017 respectively.

After having optimized pLoading and pBackground, we continue on to find the pair of pCTD and pFID that best matches the experimental release curves under control conditions. We used a similar Monte Carlo simulation approach looking at all pairwise combinations of pCTD and pFID both between 0 and 0.7 in 0.001 increments. Using experimental data, we calculated that at the moment of washout, the ratio of free radioisotopes to isotopes in folded protein to isotopes in nascent polypeptides to be 300:20:1. As such, for each pairwise simulation, we initiated the initial state of the Markov chains to reflect that ratio. For each pairwise simulation, we simulated between 15,000-20,000 Markov chains, and tracked the progression of the isotopes for 1800 seconds. The simulated proportion of radioisotopes at any point of time that is extracellular was calculated as our simulated release curve. We searched for the pair of pCTD and pFID that produced the minimum total squared error at each time point from 1-1800 seconds between the simulated curve and the observed control release curve. The optimal values for pCTD and pFID were observed to be 0.047 and 0.0 respectively.

We conducted the same optimization process of pCTD and pFID under KCl stimulation to in a manner that mirrored the above approach. We evaluated a parameter space for pCTD and pFID both between 0 and 0.2 in 0.05 increments. We searched for the pair that produced the minimum total squared error at each time point from 1-600 seconds between the simulated curve and the observed KCl release curve. The optimal values for pCTD and pFID were 0.165 and 0 respectively.

### Example 1

20S proteasome subunits are localized to neuronal plasma membranes.

Previous studies have identified localization as a key feature in determining proteasome function16. Distribution of the 26S proteasome in the nervous system has been measured using fluorescently-tagged 19S cap subunits or electron cryotomography (Cryo-ET). While cryo-ET approaches are theoretically unbiased, the processing methods inherently select for analysis of larger complexes, and therefore are more likely to identify singly- and doubly-capped proteasomes. In order to take a high resolution and unbiased approach to evaluate localization of all proteasomes (20S and 20S-containing) in the nervous system, we performed an immunogold electron microscopy (Immuno-EM) analysis of hippocampal slice preparations using antibodies raised against either the proteasome β2, β5 or β2 subunits. These are core 20S proteasome subunits common to all catalytically active proteasomes.

We first performed western blot analysis of mouse brain lysates to assess the antibodies used for our immuno-EM studies. Brains from P30 mice were lysed and prepared for SDS-PAGE, and then immunoblotted using proteasome β2, β5, and α2 subunit antibodies. Each antibody recognized a single band by western analysis at the appropriate molecular weight (FIG. 1a-e). We proceeded to perform immuno-EM from mouse hippocampal sections using these antibodies and appropriate gold-conjugated secondary antibodies. We did not detect any significant staining using secondary gold-conjugated antibodies alone (data not shown). We observed diverse subcellular and cytosolic distribution of gold particles corresponding to proteasome subunits, as previously reported' (FIG. 1a-e and Supplementary FIG. 3a-c). Unexpectedly, we observed ˜40% of all gold particles localized to neuronal plasma membranes (PM). Similar results were obtained using two additional antibodies raised against β2 and β5 subunits, but directed against different epitopes (FIGS. 1b, d). In contrast, we did not observe PM localization of gold particles when using antibodies raised against 19S cap proteins Rpt5 or S2 subunit (FIG. 1f). Immunostaining using these 19S antibodies show diffuse cytosolic localization, consistent with prior studies¹⁰.

Extending these findings, we performed immuno-EM analysis from mouse primary neuronal cultures, as these preparations are largely devoid of non-neuronal cell types and can provide higher resolution analysis^{20,21}. No immunogold label was observed in samples treated with secondary gold-conjugated antibodies alone (data not shown). Using proteasome β2 and β5 subunit antibodies in mature cultured neurons, we observed ˜40% of immunogold signal at neuronal PMs (FIG. 2a). Of those particles observed at neuronal PMs, 43±2% overlaid PMs, 38±1.7% were located at the intracellular face, and 19±2.4% were at the extracellular face (FIG. 2a). Using similar immuno-EM approaches, we did not observe PM localization of proteasomes in cultured non-neuronal HEK293 cells, which had particles localized to the cytoplasm (data not shown). Because conjugation of a primary antibody to a gold-particle tagged secondary antibody can result in the gold particle being localized up to ˜20 nm from the target antigen, we quantified the fine localization of gold particles near neuronal PMs and plotted each particle in relation to its distance from the PM. This was a linear measurement taken from the center of the PM to the centroid of the gold particle. A majority of particles overlaid the PM, with the particle density diminishing as a function of distance from the membrane (FIG. 2b). Thus, the signal observed at plasma membranes corresponds to a unique pool of membrane-localized proteasome subunits rather than a reflection of intracellular proteasome subunits. Since core proteasome subunits are not known to be present in the cell separate from the macromolecular proteasome complex, these data likely reflected the membrane localization of intact proteasomes.

### Example 2

Neuronal membrane proteasomes are exposed to the extracellular space

Immuno-EM staining with a previously validated antibody raised against the cytoplasmic domain of the voltage-gated potassium channel, Kv1.3, only showed cytosolic labeling and labeling on the intracellular face of the PM as previously described²² (data not shown). By immuno-EM analysis we see 20S proteasome staining on the extracellular face of the PM, which raises the possibility that proteasomes may be exposed to the extracellular space (FIG. 1a-e). We decided to use three additional approaches to substantiate these findings: one specifically detecting proteasome subunits (antibody feeding) and two unbiased approaches to detect surface exposed proteins (surface biotinylation & protease protection) (FIG. 2c). First, we used antibody feeding onto live neuronal cultures23,24. No staining was observed using secondary alone controls (data not shown). Feeding a primary antibody against an N-terminal extracellular epitope of the GluR1 (N-GluR1) ionotropic receptor showed punctal staining as previously reported25. We did not observe staining upon feeding an antibody against intracellular protein MAP2 (FIG. 2d). Using the proteasome β5 subunit antibody, we observed punctal localization that was largely eliminated upon pretreatment of the β5 antibody with the β5 blocking peptide (FIG. 2d).

To biochemically determine whether proteasomes were surface-exposed, we turned to previously described surface-biotinylation/purification approaches^{26,27} followed by immunoblotting with antibodies against Actin, GluR1, Rpt5 and 20S proteasome subunits. As expected, in our streptavidin pulldown samples from surface-biotinylated neurons we did not detect cytosolic Actin and did detect GluR1 (FIG. 2e). Consistent with 20S proteasomes being surface-exposed, we detected core 20S proteasome subunits in our streptavidin pulldown but did not detect significant pulldown of Rpt5 (FIG. 2e). Several measurements were taken to assure our results were not due to poor cell health or enhanced cell permeability (data not shown).

As an orthogonal method of identifying surface exposed proteins, we used a protease protection assay, which relies on the proteolysis of extracellularly exposed epitopes of proteins upon treatment of live cells with an extracellular protease^{28,29}. Cultured cortical neurons were treated with Proteinase K (PK) for varying times and then fractionated into either cytosolic or membrane fractions. By immunoblot analysis, we found that proteasomes fractionated to the membrane, similar to N-GluR1, and were susceptible to proteolysis by extracellular PK (FIG. 2f). In contrast, proteasomes from the cytosolic fraction, similar to Tubulin, were protected from protease cleavage³⁰ (FIG. 2f). Because PK, when added to live cells can only degrade proteins exposed to the extracellular space, we interpreted this observation to mean that proteasomes were surface-exposed and that the majority of proteasomes in our membrane preparations are from plasma membranes and not from other membrane organelles. This result was corroborated using Concanavalin-A (ConA), a lectin binding protein that has been used to enrich plasma membranes³¹ (data not shown). Taken together, these data support the existence of a surface exposed proteasome complex at the neuronal plasma membrane. For convenience, we will henceforth refer to the proteasome localized to the neuronal plasma membrane as the neuronal membrane proteasome, or NMP.

### Example 3

Neuronal membrane proteasomes are tightly associated with plasma membranes

We wanted to further enhance our biochemical understanding of how proteasomes, as largely hydrophilic complexes, could be localized to the hydrophobic PM. Neuronal membranes were isolated and sequentially extracted with increasing concentrations of digitonin to pull out increasingly hydrophobic proteins. Samples were prepared for western analysis (FIG. 3a). Quantification of these immunoblots revealed that a significant percentage of alpha and beta subunits co-fractionated with cytosolic proteins (Tubulin) and hydrophobic membrane proteins (GluR1). These data are consistent with proteasomes fractionating in two different modes, one that is cytosolic and another that is membrane-bound, providing additional evidence for a unique pool of membrane-localized proteasomes in contrast to cytosolic proteasomes (FIG. 3a). To determine whether NMPs were tightly or peripherally associated with plasma membranes, we used sodium carbonate extraction. Neuronal cultures were separated into cytosolic, peripherally-associated (carbonate-soluble) and tightly-associated (carbonate-insoluble) membrane protein fractions29. Calregulin32 was used as a marker of peripherally-associated membrane proteins, whereas GluR1 was used as a marker of tightly-associated membrane proteins. Immunoblotting these fractions showed that core 20S proteasome components were tightly-associated (carbonate-insoluble), while Rpt5 was peripherally-associated (carbonate-soluble) (FIG. 3b).

We considered there were two primary ways this could be possible: (1) the proteasome itself was hydrophobic in some way or (2) the proteasome was tightly associating with integral membrane proteins. In an attempt to distinguish between these possibilities, we performed Triton X-114 (TX114) phase partitioning of cultured neurons to separate hydrophilic and hydrophobic proteins33. Immunoblotting the TX114-rich and TX114-free fractions, we observed Actin fractionated into the TX114-free phase, multi-pass transmembrane protein GluR1 fractionated into the TX114-rich phase, and EphB2, a single-pass transmembrane protein fractionated into both phases (FIG. 3c). Proteasome subunits fractionated in both phases, with only ˜20-30% of proteasome subunits fractionating in the TX114-rich phase (FIG. 3c). Based on our immuno-EM, surface biotinylation, and membrane fractionation data, up to 40% of proteasome subunits were plasma membrane-localized. We reasoned that the discrepancy between these analyses might be due to the fact that proteasomes were not sufficiently hydrophobic to exist in the plasma membrane independent of auxiliary membrane proteins.

### Example 4

Neuronal membrane proteasomes are largely a 20S proteasome and in complex with GPM6 family glycoproteins.

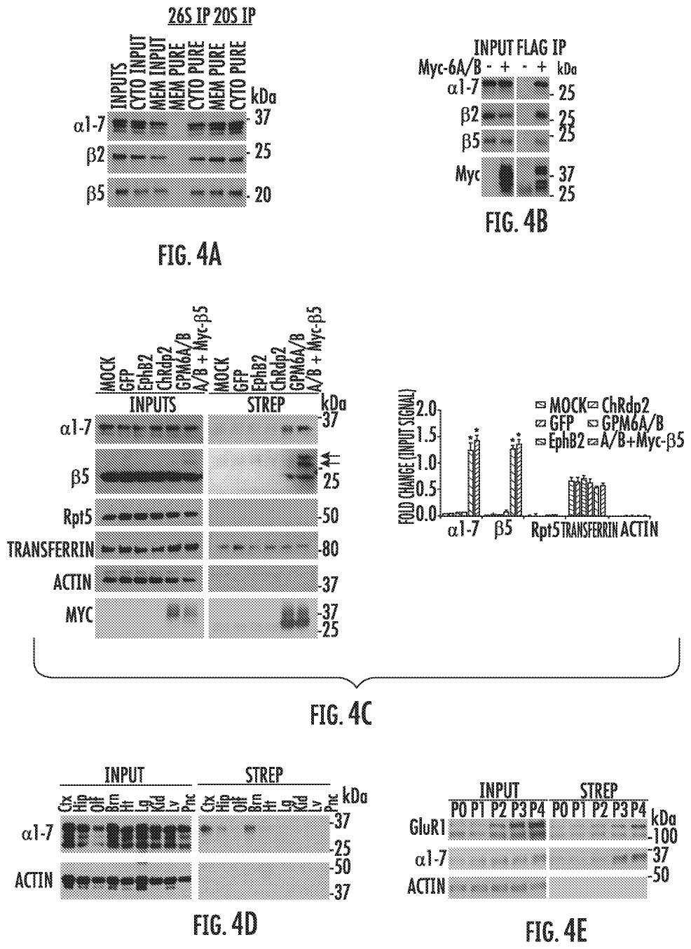

To identify potential auxiliary membrane proteins associated with the NMP we isolated proteasome complexes out of neurons using two different affinity methods³⁴. Cytosolic and membrane-extracted fractions from neuronal cultures were incubated with 20S purification matrix (purifies any 20S-containing proteasome complex) or 26S purification matrix (only purifies 26S cap-containing proteasome complex). Immunoblot analysis revealed that both 20S and 26S affinity purification matrices isolated cytosolic proteasomes, but only the 20S purification matrix was able to purify proteasomes out of the membrane (FIG. 4a), suggesting to us that this is an approach for purifying the NMP.

Using the 20S-purification matrix, we purified 20S proteasomes from the cytosol and membrane of neurons for in-depth mass spectrometry (MS) analysis. As expected, we identified all of the core 20S proteasome subunits in the purification from both membranes and cytosol (data not shown). While we identified a variety of regulatory cap proteins to co-purify with the cytosolic proteasome, we identified very few to co-purify with the proteasome purified from membranes (data not shown). These findings were validated by extensive western analysis (data not shown).

We sought to identify auxiliary membrane proteins in our MS data sets that may be capable of mediating proteasome association with the plasma membrane. We postulated that such a protein would specifically associate with the NMP compared to the cytosolic proteasome, be highly expressed in the nervous system, and be transmembrane (Supplemental Table 1b). Based on these criteria, we focused our efforts on the neuronal membrane glycoprotein GPM6A, a known member of the Proteolipid Protein family of multi-pass transmembrane glycoproteins^{35,36}. To validate these mass spectrometry data, we turned to HEK293 cells as a non-neuronal heterologous system that does not express the NMP (data not shown). Lysates from HEK293 cells previously transfected with expression plasmids encoding myc-/FLAG-tagged GPM6A and GPM6B (myc/FLAG-GPM6A/B) were immunoprecipitated using an anti-FLAG antibody. Immunoblotting using antibodies against myc and 20S proteasome subunits, we found that endogenous proteasome subunits from HEK293s co-immunoprecipitate with myc/FLAG-GPM6A/B (FIG. 4b). While we interpret these data to mean that proteasomes can associate with GPM6 proteins, as demonstrated from our MS data from neurons, we wanted to know whether the GPM6 proteins could induce the proteasome to become membrane-bound and surface-exposed. Using the surface biotinylation assay, we determined that expression of GPM6A and GPM6B in HEK293s was sufficient to induce surface expression of the endogenous HEK293 proteasome at the PM (FIG. 4c). These results are not seen upon overexpressing GFP, single-pass transmembrane protein EphB2, or multi-pass transmembrane protein Channelrhodopsin 2 (FIG. 4c). We uniformly detected the plasma membrane protein, Transferrin, verifying equal pulldown efficiency (FIG. 4c). Additionally, overexpression of myc-tagged (35 proteasome subunit together with myc/FLAG-GPM6A/B led to both myc-β5 and the endogenous subunits to become surface exposed (FIG. 4c). These findings phenocopy the phenomenon we observe in primary cultured neurons, and indicate the GPM6A/B proteins are sufficient to expose proteasomes to the extracellular space. Attempts to determine whether GPM6 family proteins were required for NMP expression were unsuccessful as shRNA-mediated knockdown of GPM6A in neuronal cultures induced cell death, suggesting GPM6 proteins may be essential for viability (data not shown).

GPM6A and GPM6B are primarily expressed in the nervous system³⁷. Consistent with these data, using our surface biotinylation assay in whole mouse tissues, we determined that NMP expression was restricted to mouse neuronal tissues (FIG. 4d). Similar results were observed using human brain tissue (data not shown). These data prompted us to determine whether NMP expression was regulated and changed over neuronal development. Using our surface biotinylation assay in slice preparations from mouse brain, we determined that NMP expression paralleled in vivo expression patterns of GluR1, whose expression functionally correlates with critical stages in neuronal development²⁶ (FIG. 4e). Performing the same experiments in neuronal cultures, we observed that the NMP was expressed in neurons at DIV8, but not prior (data not shown) in contrast to relatively constant total proteasome expression.

### Example 5

Neuronal membrane proteasomes degrade intracellular proteins into extracellular peptides (SNAPPs).

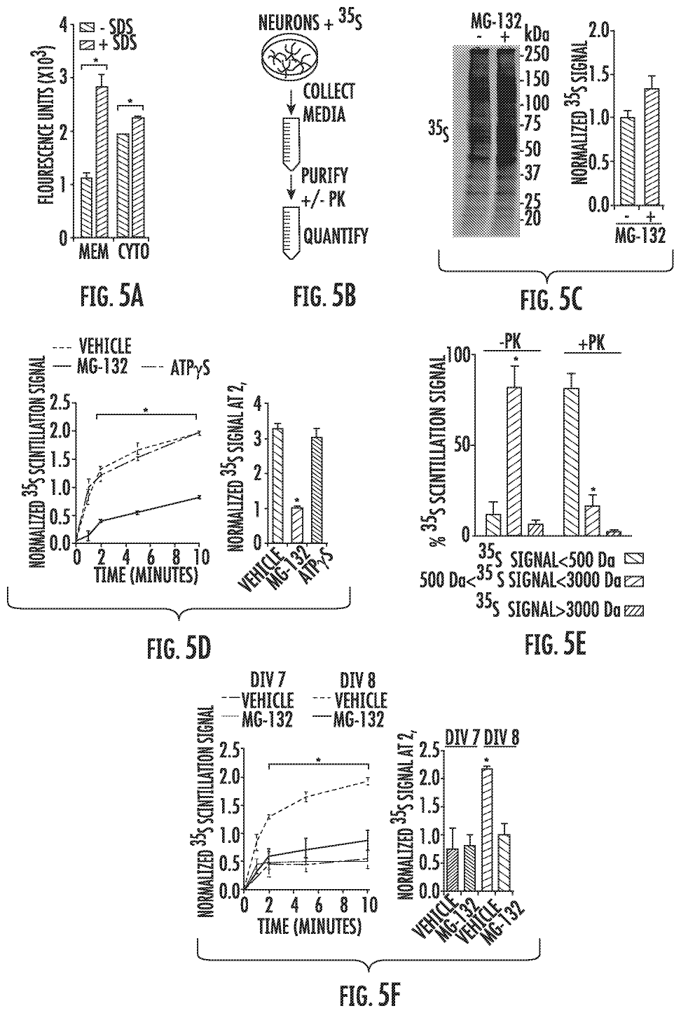

To test whether the NMP was catalytically active, we purified proteasomes from both the cytosol and neuronal plasma membranes using a 20S purification matrix and incubated them with SUC-LLVY-AMC, a substrate that fluoresces upon proteasomal chymotrypsin-like cleavage38. Addition of a low concentration of SDS to the reaction relieves the gating mechanism of the 20S proteasome without denaturing the 20S or 26S proteasome holocomplex4. Addition of SDS greatly stimulated the catalytic activity of membrane proteasomes and had little effect on cytosolic proteasome activity (FIG. 5a), consistent with a large fraction of NMPs being 20S and catalytically active.

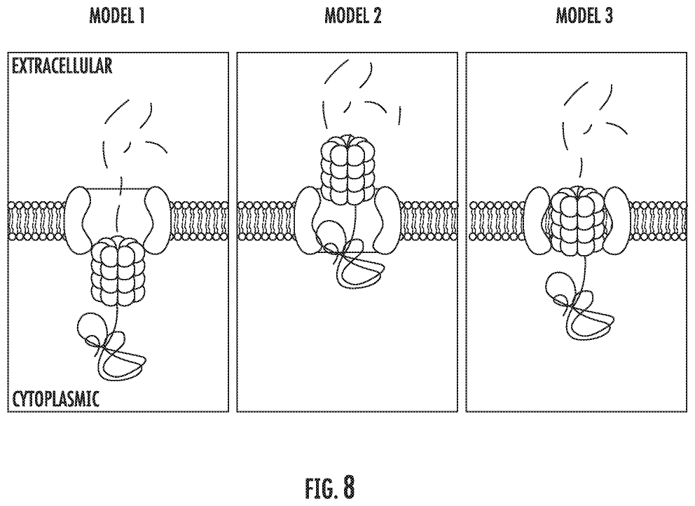

We were curious as to the purpose of a surface-exposed catalytically active 20S proteasome in the neuronal plasma membrane. Since the core 20S complex alone is ˜11×15 nm, any orientation of the NMP at the neuronal PM, which is 6-10 nm across, would provide it access to both the intracellular and extracellular space. We hypothesized that in neurons, a catalytically active proteasome in such an orientation would be able to promote proteasome-dependent degradation of intracellular proteins into the extracellular space. To test this hypothesis, we used ³⁵S-methionine/cysteine-radiolabelling approaches to trace the fate of newly synthesized intracellular proteins39 (FIG. 5b). After 10 minutes of radiolabel incorporation (FIG. 5c), free radioactivity was washed away, and media was collected over a timecourse and analyzed by liquid scintillation to detect radiolabeled proteins. We observed rapid release of radioactivity into the culture medium under baseline conditions (FIG. 5d). We observed a significant decrease in radioactive flux following addition of MG-132, without affecting radiolabelling efficiency (FIG. 5c, 5d). Addition of ATPγS, a non-hydrolyzable ATP analog, had no effect on release of radioactive material (FIG. 5d). This was consistent with the release of radioactivity being due to an uncapped 20S proteasome, which does not require ATP. To determine whether the released radiolabel was incorporated into protein peptides, different fractions from the media were treated with PK to breakdown peptidergic material into single amino acids and dipeptides. Of the released radioactive material at the 2-minute collection time, 82±5% was made up of PK-sensitive molecules that ranged between 500 and 3000 Daltons in size (FIG. 5e). Similar results were observed at a 30-minute collection time (data not shown). Since proteasome cleavage products are peptides between 500 and 3000 Da in size, we conclude that a large fraction of radioactivity in the media was composed of protein peptides derived from a proteasome40 and not individual amino acids or small molecules. To discriminate between cytosolic and membrane proteasomes in mediating the efflux of extracellular peptides, we took advantage of the temporal switch in NMP expression between DIV7 and DIV8, where both DIV7 and DIV8 neurons express cytosolic proteasomes but only DIV8 neurons express the NMP (Supplementary FIG. 7c, 7d). We observed that proteasome-dependent release of radiolabeled peptides into the media was observed at DIV8, but not at DIV7, which paralleled the temporal expression of the NMP (FIG. 5f). Consistent with this being an NMP-mediated neuronal phenomenon, we did not observe proteasome-dependent release of radiolabeled peptides in heterologous HEK293 cells that do not express the NMP (data not shown). Taken together, these data support our hypothesis that the NMP degrades intracellular proteins into extracellular peptides we call SNAPPs.

### Example 6

Neuronal membrane proteasomes are required for release of extracellular peptides (SNAPPs) and modulate neuronal activity.

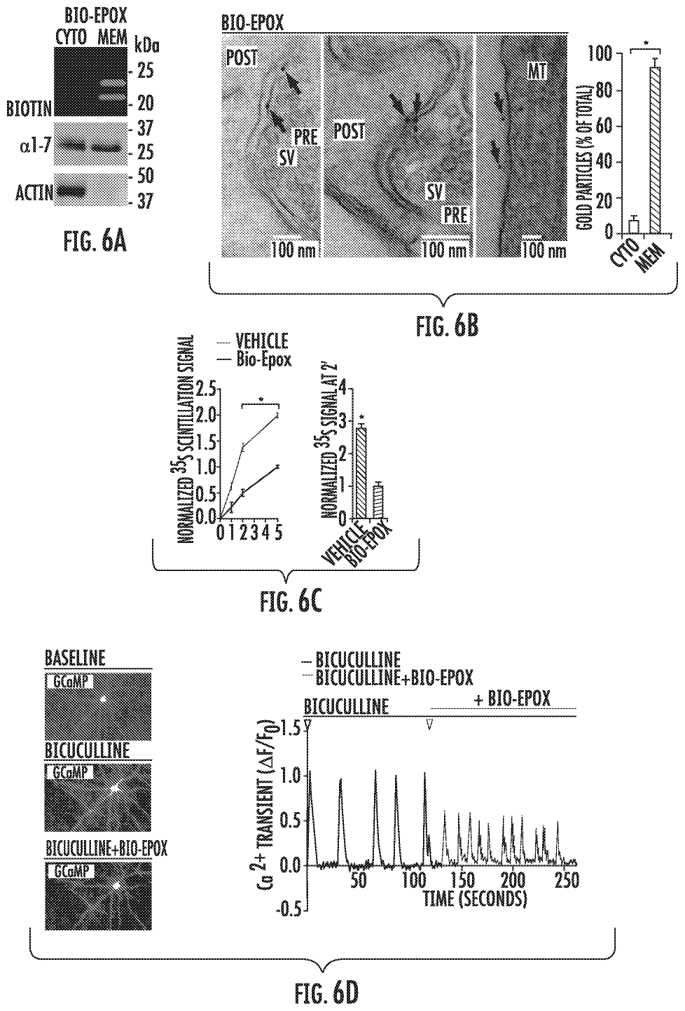

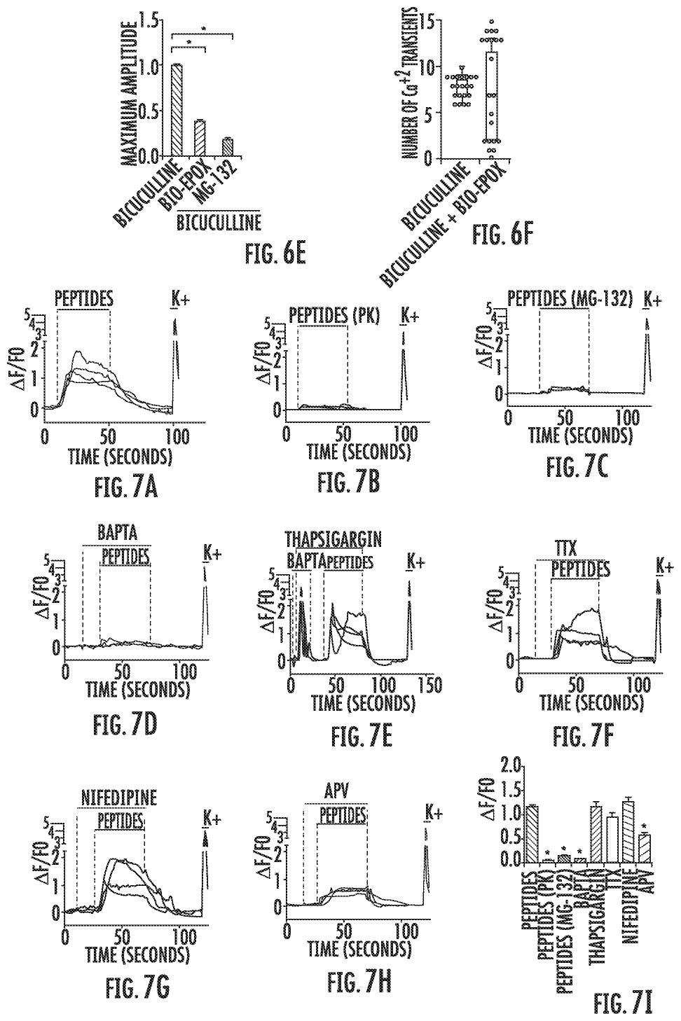

To specifically determine the contribution of the NMP in the generation of these extracellular peptides, separately from that of the cytosolic proteasome, we identified a chemical tool that was selective to the NMP. We found that biotinylation of the non-reactive portion of epoxomicin, a highly potent and specific proteasome inhibitor, generates a cell-impermeable compound (biotin-epoxomicin) that maintains target specificity⁴¹. This compound covalently modifies the catalytic proteasome β subunits, tagging them with biotin. Cultured neurons acutely treated with biotin-epoxomicin were separated into cytosolic and membranes fractions, and immunoblotted using streptavidin-AF647. Biotin signal was only observed in membranes from neurons treated with biotin-epoxomicin and at a size denoting the covalent modification of the membrane proteasome β subunits (FIG. 6a).

Furthermore, Immuno-EM analysis of neuronal cultures treated with biotin-epoxomicin showed 92±5% of biotin at plasma membranes (FIG. 6b). Any cytosolic labeling was likely due to streptavidin-Au binding endogenously biotinylated proteins, as we detected low-abundance cytosolic labeling in cultures not treated with biotin-epoxomicin (data not shown). Since biotin was directly labeled using streptavidin-Au, this analysis reduces the distance between the gold particle and the target antigen compared to conventional antibody-based immuno-EM. These data show that NMPs overlay neuronal plasma membranes and are exposed to the extracellular space and provide further evidence that the NMP is catalytically active, since epoxomicin requires proteasome activity in order to bind to and inhibit the catalytic subunits⁴². These data established biotin-epoxomicin as a useful tool for studying the relevance of the NMP.

Using this inhibitor, we sought to separate the role of the NMP from the role of the cytosolic proteasome in regulating extracellular peptide production. Acute application of biotin-epoxomicin to radiolabeled neurons inhibited radioactive peptide release into the extracellular space (FIG. 6c). Using biotin-epoxomicin, we wanted to test our initial hypothesis that the NMP could mediate rapid neuronal signaling. To test whether the NMP was relevant to aspects of neuronal signaling, changes in intracellular calcium were measured since calcium serves as a rapid readout for many types of neuronal signaling⁴³. Calcium imaging was performed using GCaMP3-transfected cultured neurons treated with perfusate containing GABAergic receptor antagonist bicuculline which, by relieving inhibition on neuronal circuits, induces regular firing of action potentials and calcium transients⁴³. Following 2 minutes of bicuculline stimulation, perfusate was switched to buffer containing both bicuculline and 25 μM biotin-epoxomicin. Within 10-30 seconds of biotin-epoxomicin addition, we observed a rapid and robust attenuation of the amplitude of bicuculline-induced calcium transients, similar to that which we observed upon acute addition of MG-132 (FIGS. 6d and 6e). Addition of biotin-epoxomicin induced a large variability in the frequency of calcium transients: 47% of neurons displayed an increase in frequency, while the same treatment induced a potent abrogation of bicuculline-induced calcium signals in 31% of neurons (FIG. 6f). Based on these data, an endogenous function of the NMP is to modulate the strength and speed of activity-dependent neuronal signaling through its proteolytic activity, possibly through the actions of the resulting extracellular peptides (SNAPPs).

### Example 7

Neuronal membrane proteasome-derived peptides (SNAPPs) are sufficient to induce neuronal signaling.

To systematically test the effects of proteasome-directed peptide signaling, peptides (SNAPPs) were purified and then perfused onto GCaMP3-encoding neurons under various conditions. Neurons were ensured to be healthy at the end of every experiment by stimulating with 55 mM KCl, which consistently induced strong calcium signaling. The proteasome-directed peptides were purified and lyophilized following extensive dialysis into ammonium bicarbonate to remove small molecules and neurotransmitters. The lyophilizate was resuspended in calcium imaging buffer. Peptide concentration was determined to be ˜50 ng/mL and was added back at that concentration. Alone, purified peptides induced a robust degree of calcium signaling in naïve neurons (FIG. 7a). This peptide-induced stimulation was eliminated if the peptide purification was done in the presence of PK (FIG. 7b). These data suggest that the observed calcium-signaling effects were due to the actions of extracellular protein peptides (SNAPPs), and not small molecules or excitatory amino acids. Moreover, media collected in the presence of MG-132 did not possess the capacity to stimulate naïve neuronal cultures (FIG. 7c), indicating that the relevant bioactive peptides were derived from the proteasome. Moreover, in similar experiments, addition of random peptides to GCaMP3-encoding neurons did not possess the capacity to stimulate naïve neuronal cultures (data not shown). We then determined that these peptides (SNAPPs) were inducing calcium flux from the outside of the cell in, rather than promoting release from intracellular calcium stores. Addition of cell-impermeable calcium chelator BAPTA to the perfusate abrogated the peptide-induced calcium signal (FIG. 7d), whereas depletion of ER calcium stores using thapsigargin did not reduce the maximum amplitude of the peptide-induced calcium signal (FIG. 7e).

To identify which channels were relevant to peptide-induced calcium activity, we used different ion channel inhibitors to pharmacologically identify relevant pathways. Blocking fast voltage-gated sodium channels using Tetrodotoxin did not block the peptide-induced calcium signal, revealing that the influx of calcium was probably not due to action potential-induced signaling, and more likely directly due to effects on calcium channels (FIG. 7f). Blockade of L-type calcium channel dependent influx using Nifedipine also did not modulate the peptide-induced calcium signal (FIG. 7g). However, inhibiting N-methyl-D-aspartate receptors (NMDARs) using 2-amino-5-phosphonopentanoic acid (APV) reduced the maximum amplitude of the peptide-induced calcium influx (FIG. 7h). Together, these data suggest that the peptides derived from the neuronal membrane proteasome (SNAPPs) can modulate neuronal activity, at least in part by driving calcium influx through NMDARs (FIG. 7i).

### Example 8

NMP-mediated mechanisms ameliorate the early events of Aβ-induced neurological decline; a first step toward NMP-directed therapeutics in treating Alzheimer's disease.

A prevailing hypothesis in Alzheimer's disease (AD) research is that amyloid beta peptide (Aβ) causes plaque formation in the brain, ultimately giving rise to neurodegeneration observed in the AD patient population. Aβ targeted therapeutics is the leading effort in the medical and pharmaceutical community aimed at ridding the world of Alzheimer's disease.

Based on data already provided we now know that the levels of NMP are significantly reduced in AD human brains, brains from AD mouse models and cultured primary neurons treated with Aβ1-42 peptide. Aβ1-42 peptide has been shown extensively both in vitro and in vivo to cause events that lead to neuronal degeneration and animal decline in behavior and physiology relevant to AD.

Based on our work, the NMP is the only enzyme complex in the nervous system that generates proteasome-derived extracellular signaling peptides. Thus, we considered that downregulated levels of the NMP in AD would lead to reduced extracellular peptide production in these patient brains. How early in the disease this occurs remains unclear. If indeed the NMP and its resulting extracellular peptides played a role in AD, two ensuing mechanisms would be possible: 1) reduced levels of the NMP would no longer turnover a certain set of intracellular proteins important for neuronal health, thereby leading to AD, or 2) reduced levels of the NMP would, by definition, lead to reduced extracellular peptide production and reduction of these peptides would make the neuron more susceptible to AD-relevant events, possibly mediated by the 36 to 42 amino acid Aβ-peptide. We favor the second possibility as we have not yet detected any significant change in the levels of any given protein through the NMP. Thus, we hypothesized that reduced NMP-derived peptide production may contribute to AD. We first considered that this could be in relation to the pathogenicity of Aβ-induced neurotoxicity. The reason for this thinking is that Aβ is an endogenous peptide and may either cooperate or compete with the endogenous NMP-derived peptides in the nervous system. To first test this hypothesis, we incubated primary neuronal cultures with fluorescently labeled Aβ1-42 with or without NMP peptides. The endogenous concentration of these peptides is 250 ng/mL, which is approximately 250 nM. We performed a titration of NMP-derived peptides together with a constant concentration of Alexa Fluor 488-labeled Aβ. This labeled Aβ has been previously shown to interact with neurons in a manner that leads to neurodevelopmental decline. It is a surrogate for investigating how Aβ interacts with neurons, the very first step that leads to cognitive decline in AD. We identified that increasing concentrations of NMP-derived peptides led to a reduction in Aβ binding to neurons, with half-maximal effect observed at the endogenous concentration of NMP-derived peptides (FIG. 10). We interpret this to mean that NMP-derived peptides competed with Aβ in a dose-dependent manner.

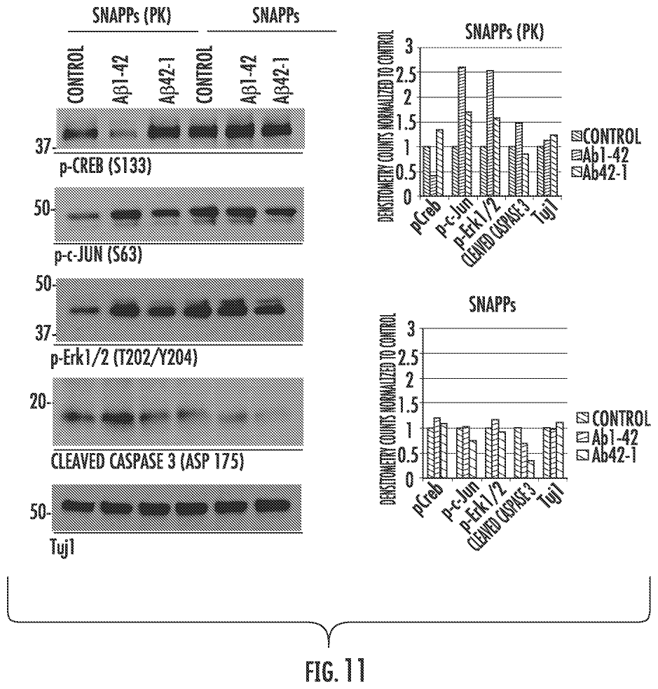

Because NMP peptides could compete away Aβ binding, we hypothesized that this competition might lead to a reduction of Aβ-induced neurotoxic effects. While there are many measures of Aβ-induced toxicity, we were primarily interested in the widely accepted Aβ-induced effects on signaling which are thought to be initiating stimuli to the onset of neurodegeneration in AD. These include decreased phospho-CREB, elevated phospho-c-Jun, elevated phospho-Erk1/2, and elevated cleaved caspase-3 [Vitolo et al 2002 PNAS, Morishima et al 2001 J Neurosci, Chong et al 2006 JBC]. We replicated all of these effects upon treatment of primary neuronal cultures with Aβ1-42. When neurons were incubated with Aβ1-42 and then treated with NMP-derived peptides, we observed that neurons were insensitive to Aβ effects on intracellular signaling. As a control treatment, we compared samples treated with NMP-derived peptides to samples treated with NMP-derived peptides pre-treated with proteinase K (PK), which destroys the peptides. Consistent with previous data, we did not observe any molecular phenotypes when treating neurons with the reverse Aβ42-1 peptide in comparison to Aβ1-42 (FIG. 11; compared lanes in SNAPPs (PK) treatment). It is important to note that NMP-derived peptides (SNAPPs) alone have an effect on most of the signaling pathways that we evaluated (FIG. 11; compare control lanes between SNAPPs (PK) and SNAPPs). However, provided that we have seen that NMP-derived peptides can stimulate neurons, these data are unsurprising. Together, we interpret these data to mean that the addition of NMP-derived peptides can prevent Aβ induced effects on critically important intracellular signaling pathways relevant to AD. This is likely due to the NMP peptides blocking Aβ binding to neurons.

Collectively, these data support the hypothesis that that NMP peptides can serve as endogenous blockers or inhibitors of Aβ binding to neurons. They are the first data of their kind to demonstrate the existence of an endogenous inhibitor of Aβ binding. Moreover, they provide promise that exogenously elevating NMP-derived peptide levels or in theory, chemically inducing NMP levels to enhance endogenous peptide production, may both be viable therapeutic approaches to reverse molecular phenotypes in AD. It should be noted, that because the NMP is specific to the nervous system, targeted approaches to this system should have minimal off-target effects. We expect that NMP-directed pathways will serve as critical players in the hunt to identify new avenues for reversing AD phenotypes in intact systems, efforts which we are currently undertaking.

Using parameters determined in the above experiments, we constructed Markov process chain models in silico which predicted that the kinetics of this process necessitate coordination of translation and degradation. In a series of biochemical analyses, this predicted coordination was instantiated by NMP-mediated and ubiquitin-independent degradation of ribosome-associated nascent polypeptides. Using in-depth, global, and unbiased mass spectrometry, we identified the nascent protein substrates of the NMP. Among these substrates, we found that immediate-early gene products c-Fos and Npas4 were targeted to the NMP during ongoing activity-dependent protein synthesis, prior to activity-induced transcriptional responses. The following examples provided herein generally define an activity-dependent protein homeostasis program through the NMP that selectively targets nascent polypeptides prior to adopting their final functional conformations.

### Example 9

Neuronal stimulation induces NMP-dependent degradation of newly synthesized proteins into extracellular peptides.

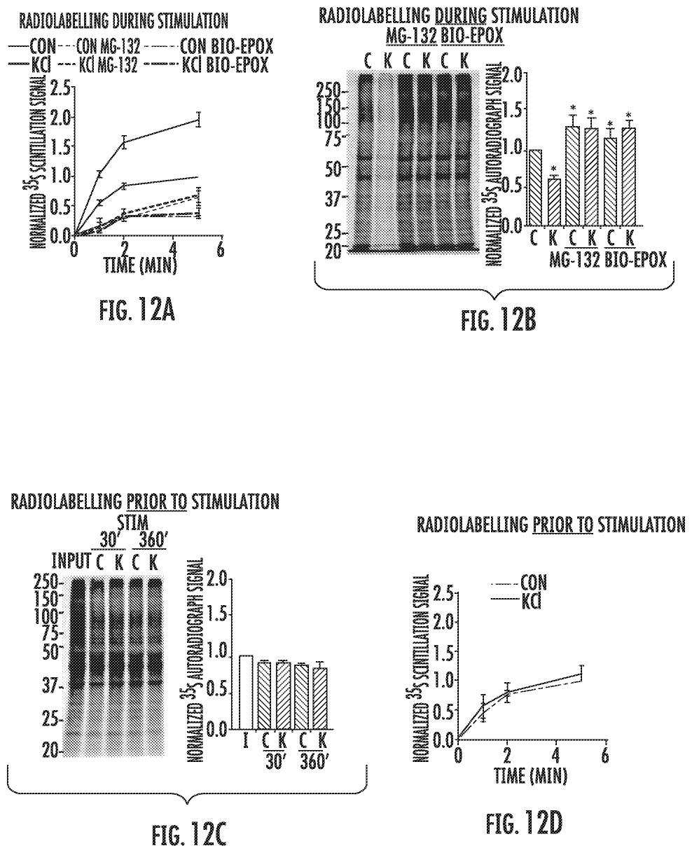

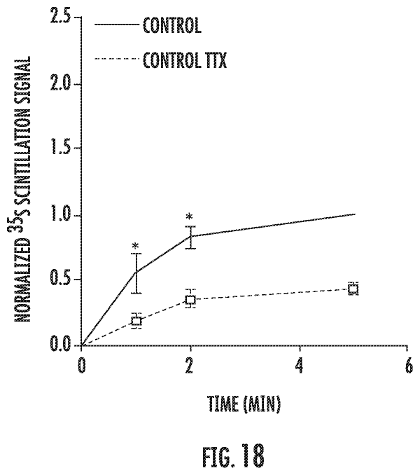

To extend our observed findings in FIG. 18 and determine whether neuronal activity induces NMP function, we monitored NMP-dependent production of extracellular peptides under states of neuronal stimulation. We first used KCl-induced membrane depolarization as a classic and effective tool to induce elevated activity of the majority of neurons in culture (Lin et al., 2008; West et al., 2001; Xia et al., 1996). Primary mouse cortical neuronal cultures at days in vitro (DIV) 10-14 were treated with either a stimulation buffer (KCl) or a control buffer (NaCl). These neurons were concomitantly radiolabeled with ³⁵S-methionine/cysteine for 10 minutes, without any prior metabolic deprivation (Ramachandran and Margolis, 2017; Vabulas and Hartl, 2005). Following concomitant radiolabeling and neuronal stimulation, we washed away both free isotope and stimulation buffer. This media was replaced with fresh conditioned media containing either a pan-proteasome inhibitor (MG-132), an NMP-specific inhibitor (biotin-epoxomicin), or control (DMSO) (Li et al., 2013; Meng et al., 1999a, b; Ramachandran and Margolis, 2017; Sin et al., 1999). Immediately following washout, samples were taken from the extracellular medium over time and analyzed by liquid scintillation. We have previously shown that this method preferentially monitors the release of extracellular NMP-derived peptides over small molecules or free isotope (Ramachandran and Margolis, 2017). We observed a significant MG-132 and biotin-epoxomicin-sensitive increase in radiolabelled extracellular peptides released from neurons that had been stimulated, compared to controls (FIG. 12A). These data were consistent with the released material being comprised of protein peptides derived from the NMP (Ramachandran and Margolis, 2017).

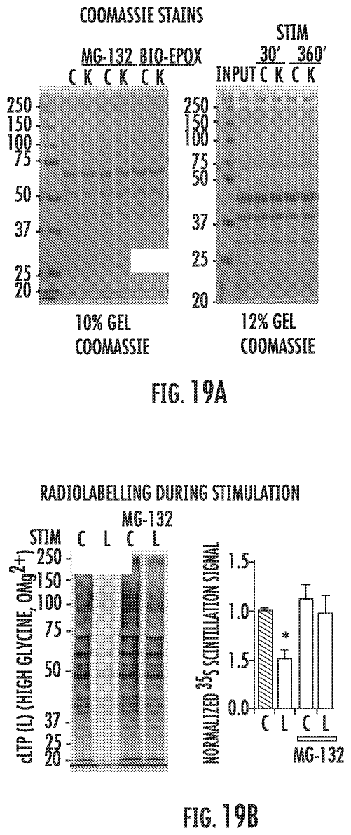

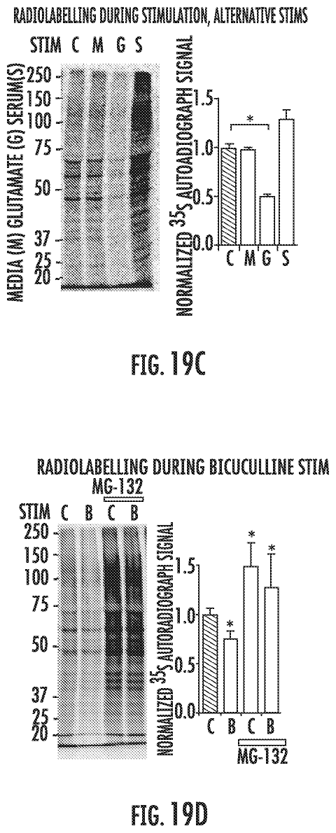

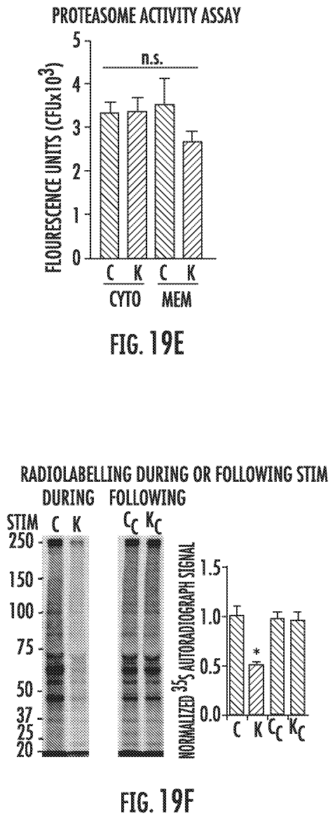

Our working hypothesis was that the observed stimulation-induced NMP-dependent increase in extracellular peptide production would be reflected in enhanced NMP-mediated degradation of a pool of intracellular protein substrates. To test this, we measured the intracellular pool of proteins made during elevated neuronal activity using SDS-PAGE and autoradiography. Neurons were treated with the radiolabeling protocols described above. All samples were coomassie stained after SDS-PAGE to ensure equal sample loading (FIG. 19A). By densitometry analysis of these autoradiographs, we noticed a decrease in radioactive intracellular protein signal from neurons that had been radiolabelled during stimulation (FIG. 12B). This effect was induced by a variety of well-characterized stimulation protocols that give rise to activity-dependent neuronal signaling, but not by serum containing growth factors (FIGS. 19B-D)(Fortin et al., 2010; Lin et al., 2008; Marin et al., 1997; Scheetz et al., 2000). Treating these neurons with MG-132 or biotin-epoxomicin during radiolabelling blocked the stimulation-induced loss of radiolabelled protein signal (FIG. 12B). We interpret this to mean that neuronal activity enhances NMP-mediated degradation of intracellular proteins made during stimulation. This enhanced degradation of intracellular substrates was not due to increased intrinsic catalytic activity of the NMP (FIG. 19E).

Our experiments thus far monitored the NMP-mediated and activity-dependent turnover of proteins made during stimulation. Given that certain protein populations have been shown to be more susceptible to degradation than others (Ha et al., 2016; McShane et al., 2016; Wheatley et al., 1980), we asked whether the degradation kinetics for proteins synthesized during stimulation were different than those for proteins made prior to or following stimulation. Surprisingly, by changing our radiolabeling protocols, we did not observe the same magnitude of stimulation-induced degradation of proteins from neurons that had been radiolabelled prior to the onset of stimulation, even after sustained stimulation (FIG. 12C). Consistent with this, we also did not detect a stimulation-induced increase in extracellular radioactive peptide efflux when neurons were radiolabeled prior to, instead of during stimulation (FIG. 12D). Additionally, we observed no change in intracellular radiolabelled protein signal from neurons that had been radiolabelled immediately following stimulation (FIG. 19F). These data illustrate that neuronal stimulation does not simply promote the turnover of all proteins, but specifically enhances the NMP-mediated turnover of newly synthesized proteins made during neuronal stimulation.

### Example 10

Monte Carlo simulation of Markov chains favors degradation of nascent polypeptides as the source for NMP-derived extracellular peptides.

Our understanding of NMP function was that it directly degrades intracellular proteins into peptides in the extracellular space (Ramachandran and Margolis, 2017). This predicts that degradation kinetics of intracellular NMP substrates are directly coupled to the release kinetics of the extracellular peptides (Ramachandran and Margolis, 2017). The data thus far relied on ³⁵S-methionine/cysteine addition to neuronal cultures and tracing the fates of the proteins in which radioactive isotopes were incorporated. Following charging onto a tRNA, isotopes go through two major steps on their way to being incorporated into a folded protein: First, they must be incorporated into the growing nascent polypeptide which is associated with the ribosome during protein synthesis. Subsequently, this polypeptide must go through the complex task of folding before achieving its proper folded conformation, some of which is achieved while still ribosome-associated (Gloge et al., 2014; Hartl et al., 2011; Kramer et al., 2009; Pechmann et al., 2013). Very generally, polypeptides progressing from one stage to the next adopt increasing conformational stability with a corresponding increase in their half-lives (Alberts B, 2002). We sought to understand whether our data revealed any selectivity by which population of polypeptides (i.e. nascent polypeptide, folding intermediate, or folded protein) were being targeted for degradation by the NMP.

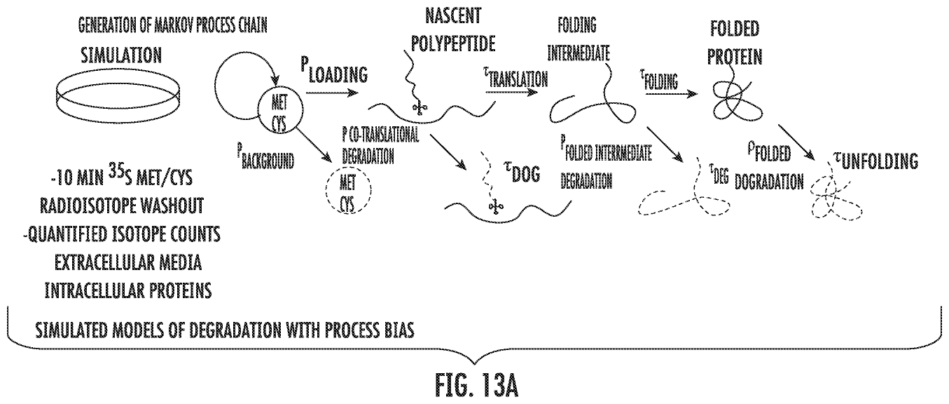

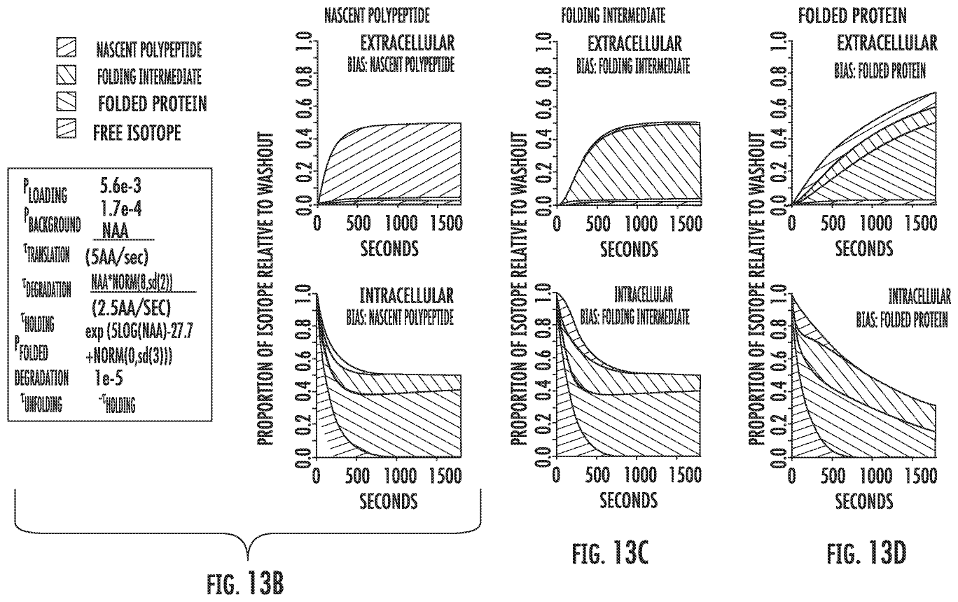

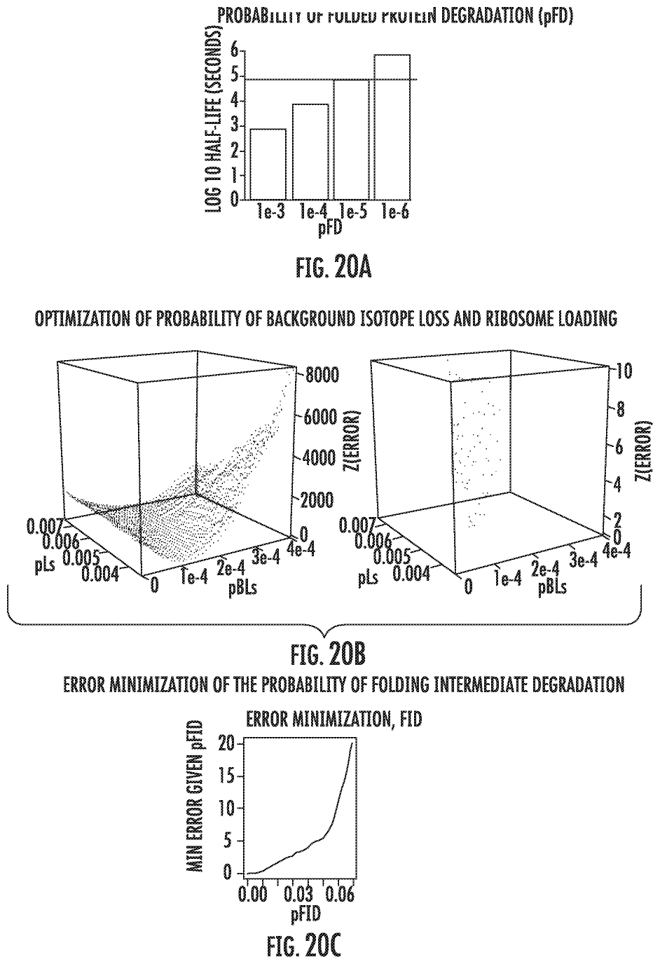

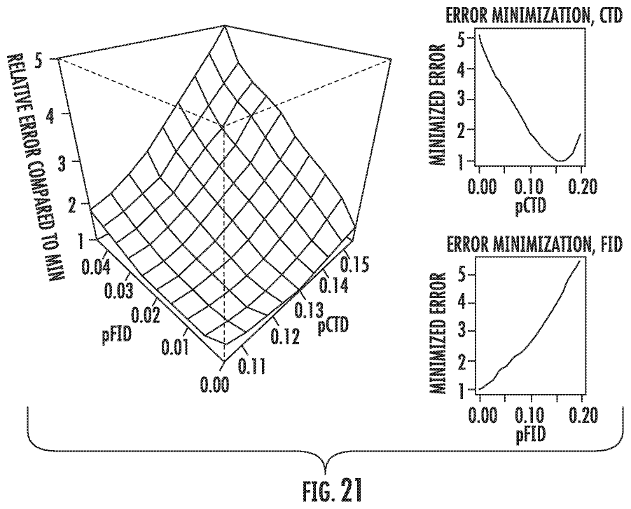

To achieve this goal, we constructed a simplified Markov chain model to track the fate of radioisotopes over a time course that mirrors our experimental peptide release data. Each Markov chain follows the trajectory of a single radioisotope that begins as a free radioisotope inside the cell, following 10 minutes of simulated isotope incorporation (FIG. 13A). The radioisotope can progress from the initial free state to become incorporated into a nascent polypeptide, and then into a folding intermediate, and finally into a folded protein. In each of these four possible states of incorporation, the radioisotope has some probability of extracellular release (FIG. 13A). The transition probabilities from one state into the next and the release mechanisms at each state are modeled after well-established kinetic parameters (e.g. rates of protein translation, degradation, and protein folding) and take into account the distribution of protein sizes in neurons (FIG. 13C and 20A)(Balchin et al., 2016; Hartl et al., 2011; Lane and Pande, 2013; Pande, 2014; Wu et al., 2016). By representing a single experiment as a collection of Markov chains, we could model the proportion of radioisotopes that are either inside or outside of the cell at any point in time. These simulated values for extracellular radioisotope release were evaluated against our experimentally observed release curve. We took the diffusion of free isotope into account by optimizing our model against radioisotope release when all proteasomes are inhibited by MG-132 (FIG. 20B).

While our model was simple, we attempted to account for as many factors as reasonable using biologically determined parameters. When the model was biased towards turnover of nascent polypeptides, we observed that the shape of the in silico release curve closely mirrors the shape of the experimental release curves (FIG. 13B). The direct degradation of nascent polypeptides by a proteasome is the operational definition of co-translational degradation(Duttler et al., 2013; Inada, 2017; Kramer et al., 2009; Wheatley et al., 1982), which is how we will refer to this process. In contrast, by shifting the bias towards turnover of folding intermediates, the simulated release curves followed a sigmoidal shape. Although this curve can match the experimental release curve at 5 minutes and beyond, these data considerably underestimate values for any time span less than 5 minutes (FIG. 13C). More dramatically, biasing the model towards turnover of folded proteins generated a continually gradual and linear release curve. This indicated a rate far too slow to account for the rapid release and subsequent taper of experimentally released radioisotopes (FIG. 13D).

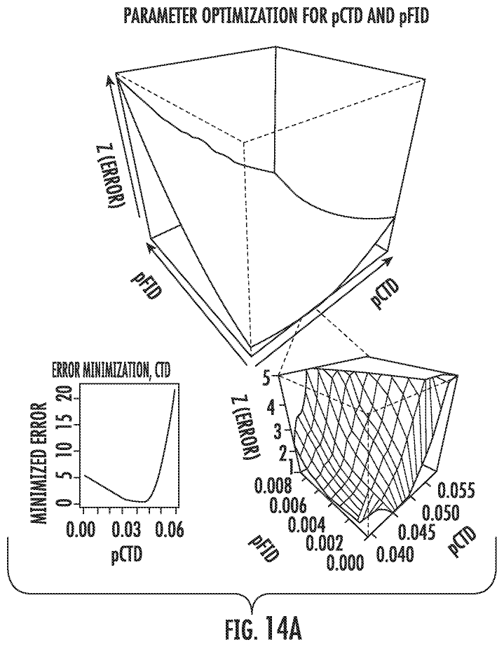

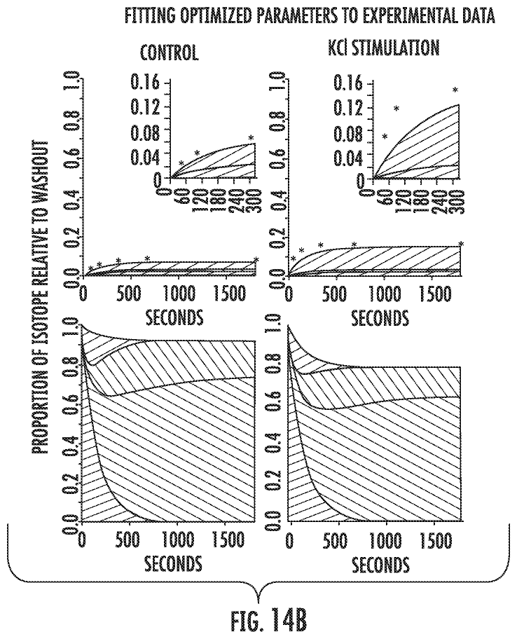

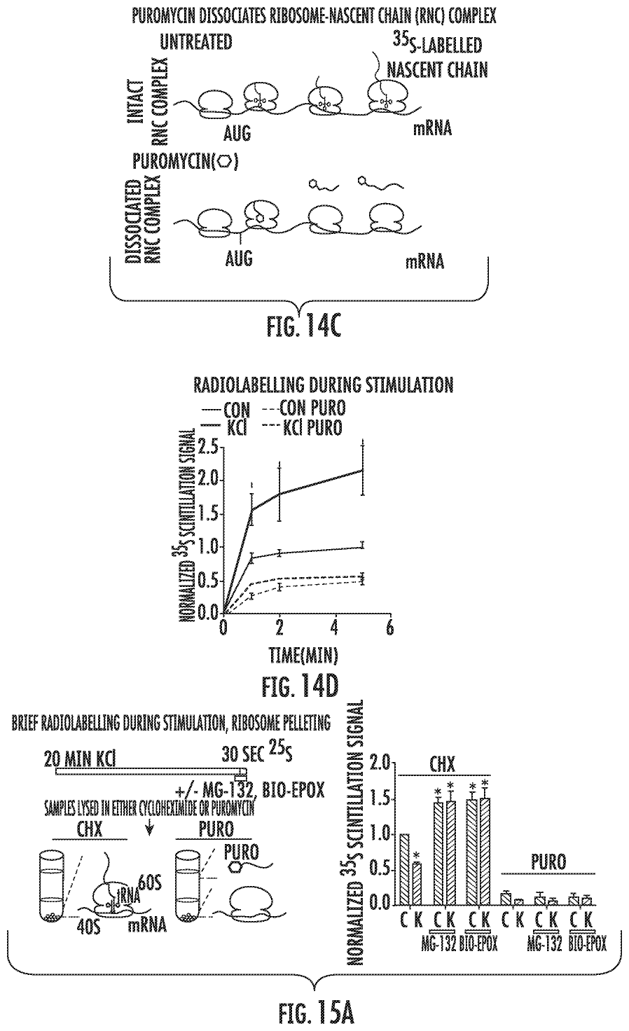

The shapes of the release curves for co-translational degradation and folding intermediate degradation more closely approximated our experimental data than those for folded protein degradation. To further refine our analysis, we used Monte Carlo simulations to optimize which combinations of the probabilities for co-translational and for folding intermediate degradation best give rise to the observed release data (FIG. 14A). We sampled a large parameter space of possible pairwise probabilities, and for each combination of co-translational and folding-intermediate degradation probability, we simulated a large number of Markov chains and calculated each predicted release curve. By minimizing the error of the predicted curves against the experimental data, we could identify a set of probabilities that most closely mirrored our experimental data. We began performing calculations using the release data from control-treated neurons. In this condition, the error between the simulated and observed data was minimized at values corresponding to 0% folding intermediate degradation probability, and a probability of 4.7% that a nascent polypeptide would be targeted to co-translational degradation in a one second time window (FIG. 14A, 20C). These values favoring degradation of nascent polypeptides give rise to a simulated release curve that exhibits the rapid logarithmic rise and gradual taper of released radioisotopes with minimal discrepancy to the experimental release curve (FIG. 14B). By increasing the co-translational degradation probability from 4.7% to 16.5%, we minimized error against the experimental KCl stimulation data more efficiently than by modifying the probability of folding intermediate degradation (FIG. 14B, 21). This also simulated decreased intracellular protein to a similar magnitude to what we observed in our experimental data (FIG. 12B, 13E). We conclude from these models that the most likely explanation for our experimental release data is that neuronal stimulation enhances the rate of co-translational degradation. We next sought to experimentally test this prediction made by the Markov model.

Co-translational degradation requires translation elongation (Duttler et al., 2013; Inada, 2017; Kramer et al., 2009; Wheatley et al., 1982). One of the hallmarks of co-translational degradation is its sensitivity to the translation elongation inhibitor puromycin (Nathans, 1964). Puromycin is an aminoacyl-tRNA structural analog that engages into the peptidyl transferase center of the ribosome and covalently modifies the growing polypeptide (FIG. 14C)(Nathans, 1964; Nathans and Neidle, 1963; Shao et al., 2013; Wang et al., 2013). This specifically disrupts translation elongation by dissociating the growing nascent polypeptide from the ribosome. Treatment of neurons with puromycin following concomitant radiolabeling and neuronal stimulation resulted in a significant reduction of NMP peptide release from both KCl-stimulated and control neurons (FIG. 14D). These data support the prediction made by our modeling data that translation elongation was required for the production of NMP-derived extracellular peptides. Collectively, these data provide evidence that nascent polypeptides were co-translationally degraded by the NMP into extracellular peptides.

### Example 11

Neuronal stimulation induces NMP-mediated co-translational degradation of ribosome-associated nascent polypeptides.

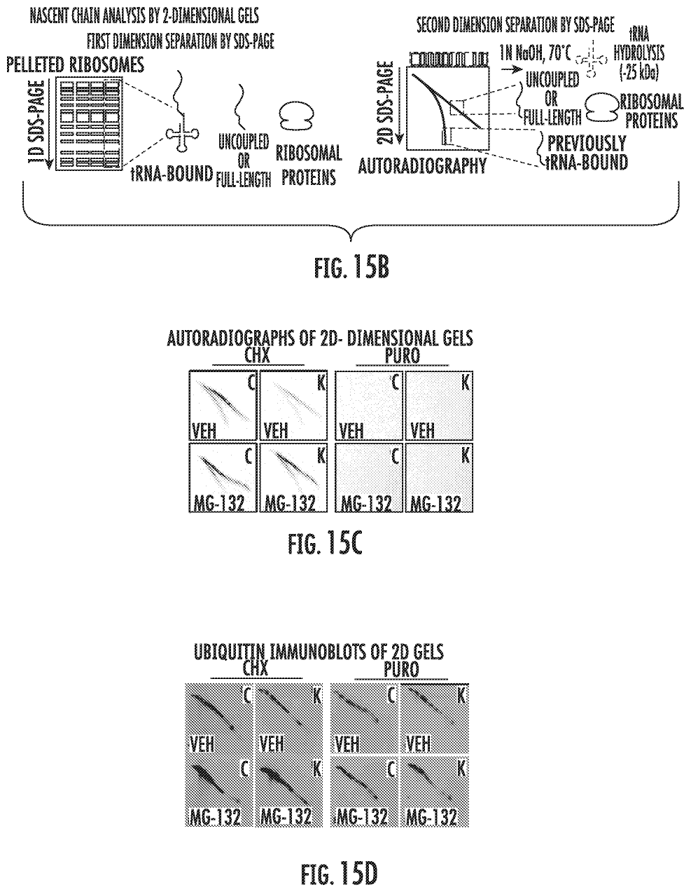

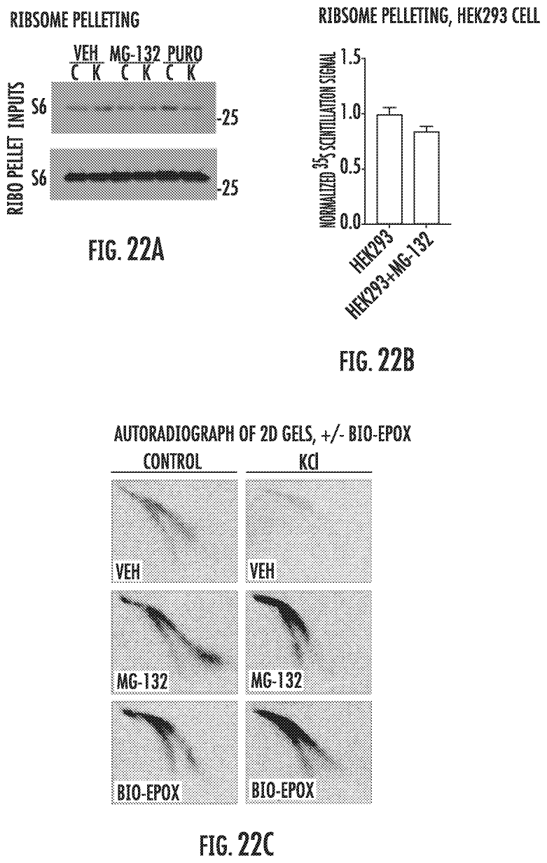

During translation elongation, nascent polypeptides are bound to a tRNA within the ribosome. This complex is collectively referred to as a ribosome-nascent chain complex (RNC)(Duttler et al., 2013). However, multiple groups have reported conditions where nascent polypeptides are separated from the RNC prior to their completion and are subsequently degraded (Duttler et al., 2013; Shao et al., 2013; Wang et al., 2013). To determine whether the NMP was targeting nascent polypeptides while still associated with the RNC, we performed ribosome pelleting assays to isolate RNCs (Brandman et al., 2012). Briefly, ³⁵S-cysteine/methionine radiolabel was added to neuronal cultures in the presence of proteasome inhibitors for only 30 seconds. This shortened protocol preferentially labels nascent polypeptides before they finish synthesis into full-length proteins (Dunler et al., 2013; Ito et al., 2011). Immediately following radiolabelling, neurons were lysed either in the presence of cycloheximide (CHX) and proteasome inhibitors to freeze translation and degradation, or with puromycin and proteasome inhibitors to release the nascent polypeptide from the ribosome and freeze degradation (FIG. 15A—model). RNCs were subsequently pelleted as previously described, with equal ribosome loading across samples (FIG. 22A). By liquid scintillation analysis of CHX-treated samples, we noticed a decrease in radioactive signal in RNC pellets from neurons that had been radiolabelled during stimulation compared to controls (FIG. 15A). Consistent with the radioactivity solely coming from the nascent polypeptide, treatment with puromycin resulted in a complete loss of radioactivity in the RNC pellet (FIG. 15A). Treating neurons with MG-132 or biotin-epoxomicin during radiolabelling blocked the stimulation-induced reduction in radioactive signal in the RNC pellet (FIG. 15A). We believed that this proteasome-mediated turnover of nascent polypeptides was neuronal-specific, as we did not observe an increase in radiolabelled signal from RNCs isolated from MG-132 treated HEK293 cells (which do not express the NMP (Ramachandran and Margolis, 2017)) (FIG. 22B). Notably, we observed a ˜20% increase in radiolabeled signal in RNCs isolated from neurons that had been treated with proteasome inhibitors (FIG. 15A).

To extend these analyses and specifically monitor nascent polypeptides separately from the RNC complex, we leveraged previously described two-dimensional gel electrophoresis (2D-gel) approaches that separate the nascent polypeptides in the form of peptidyl-tRNA from full-length proteins (Ito et al., 2011). Briefly, pelleted RNCs from neurons radiolabeled for 30 seconds were separated in the first dimension by SDS-PAGE (FIG. 15B). Next, individual gel lanes were treated with base to hydrolyze tRNAs from their bound nascent polypeptides, and subsequently separated by SDS-PAGE in the second dimension (FIG. 15B). Separating nascent polypeptides from their tRNAs shifts their molecular weight, changing the migration of pattern of these nascent polypeptides in the second dimension. Nascent polypeptides hydrolyzed from their tRNAs ran as a fast-migrating band, in stark contrast to a slow-migrating band consisting of polypeptides that were not bound to tRNA in the first dimension. This tRNA-free population was comprised of full-length proteins (e.g. ribosomal proteins) and nascent polypeptides separated from their tRNAs during processing in the first dimension (FIG. 15B). In our analysis, we found puromycin-sensitive radiolabelled signal in both the fast- and slow-migrating bands, consistent with the entire radioactive signal associated with the RNC complex being derived from the nascent polypeptide (FIG. 15C).

Using this approach, we analyzed isolated RNCs from radiolabelled neurons following KCl stimulation. We observed approximately a 40% reduction in radiolabel signal intensity of both the fast- (tRNA-hydrolyzed polypeptide) and slow-migrating bands from KCl-stimulated versus control samples (FIG. 15C). Consistent with our quantification of scintillation counts in RNCs, the stimulation-induced loss of radiolabel signal was entirely recovered by treating neurons with MG-132 or biotin-epoxomicin as described above (FIG. 15C, 22C). Immunoblotting these samples using an antibody against ubiquitin revealed detectable signal in the slower migrating band of the 2D-gel which was undetectable in the faster migrating nascent polypeptide band (FIG. 15D). Importantly, we detected ubiquitin immunoblot signal from puromycin-treated samples in the slower migrating band (FIG. 15D). Therefore, based on these data, we suggest that the nascent chain is not ubiquitylated at sufficient levels to explain the stimulation-induced turnover we observed. However, nascent chains bound to tRNA and most likely RNC-associated, are targeted for degradation. We concluded from these data that neuronal stimulation induces NMP-mediated co-translational degradation of ribosome-associated nascent polypeptides in a ubiquitin-independent manner. These data were consistent with the NMP operating as a 20S proteasome, which degrades unfolded polypeptides in an ubiquitin-independent manner (Ben-Nissan and Sharon, 2014; Coux et al., 1996).

### Example 12

Identification of activity-dependent nascent NMP substrates.

During neuronal stimulation, were all nascent polypeptides similarly susceptible to co-translational degradation or was there some selectivity in which nascent polypeptides were being targeted? To specify these principles of co-translational degradation through the NMP in an unbiased manner, we turned to global proteomic analysis. A variety of methods have been developed to analyze newly synthesized polypeptides, typically by introducing chemically modifiable noncanonical or unnatural amino acids (Aakalu et al., 2001; Dieterich et al., 2010; Dieterich et al., 2006; Landgraf et al., 2015). These are typically methionine analogs that are incorporated into newly synthesized polypeptides, and serve as a handle to isolate the polypeptides they modify (Aakalu et al., 2001; Dieterich et al., 2010; Dieterich et al., 2006; Landgraf et al., 2015). While these are powerful tools, two issues confounded our use of such approaches. First, decades of work into the stability of nascent chains and newly synthesized polypeptides has shown that proteins made with non-natural amino acids have a higher propensity to be turned over by the proteasome during or immediately following their synthesis [(Benaroudj et al., 2001; Etlinger and Goldberg, 1977; Goldberg and Dice, 1974; Prouty and Goldberg, 1972; Prouty et al., 1975; Rock et al., 2014; Rock et al., 1994; Wheatley, 2011; Wheatley et al., 1980; Wheatley et al., 1982)]. This method would likely bias our analysis of newly synthesized proteasome substrates, and provide an artificial overestimate of this population. Second, the met-tRNA that charges these amino acids prefers endogenous methionine. Therefore, to induce the incorporation of noncanonical amino acids, cells must be incubated in methionine-free media. Additionally, the charging of noncanonical amino acids on met-tRNA is slower, and the efficiency of chemical modification and purifications are imperfect (Hartman et al., 2006). To overcome these limitations, studies utilizing these techniques usually incubated cells for at least one hour in media containing noncanonical amino acids to maximize labeling. These timescales were incongruent with the timescales at which we were conducting our experiments.

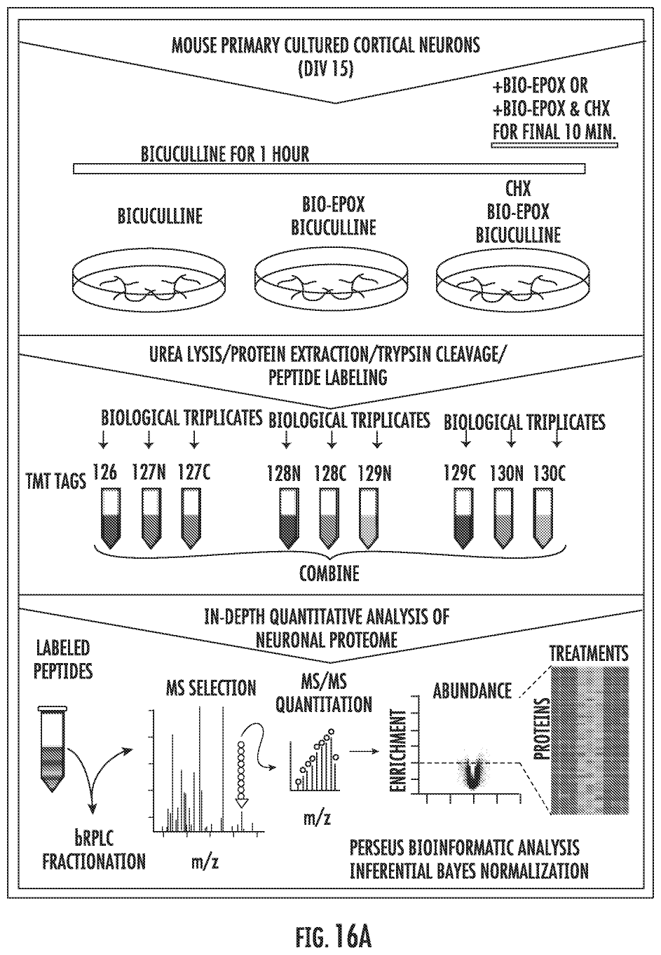

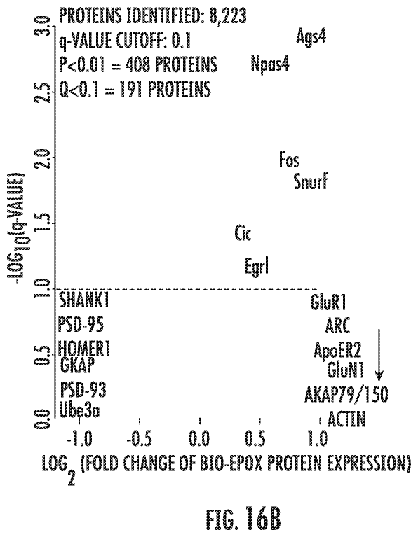

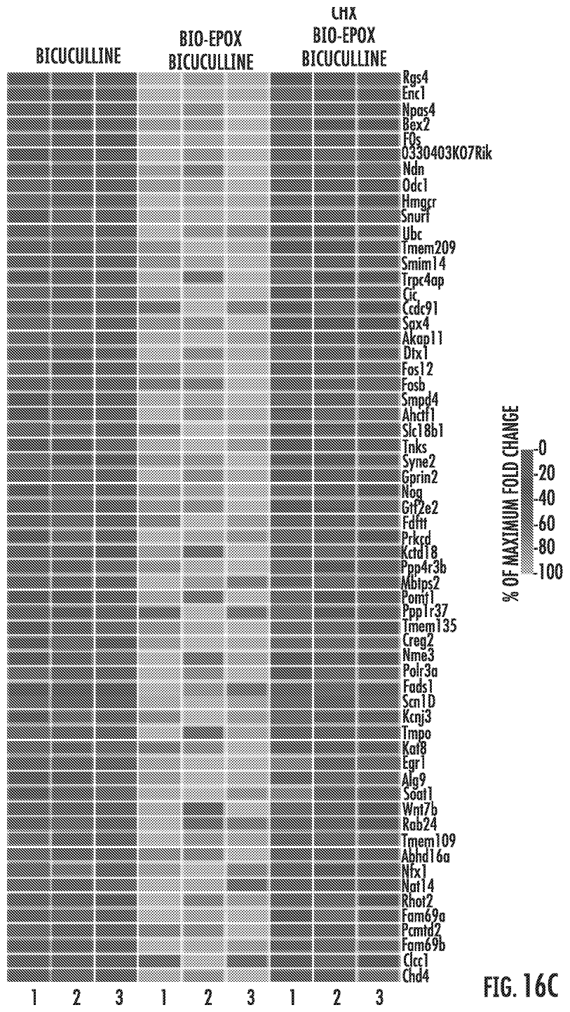

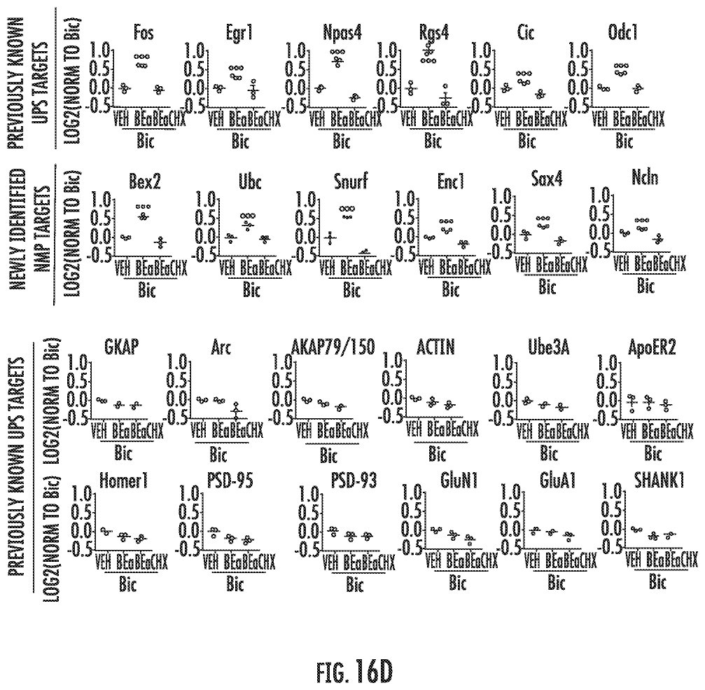

Because of the combination of these variables, we chose not to use noncanonical or unnatural amino acids to identify co-translationally degraded substrates of the NMP. Instead, we leveraged unbiased and high-coverage mass spectrometry-based quantitative proteomic analysis using tandem mass tag (TMT) technology (FIG. 16A). Primary mouse cortical neuronal cultures were incubated with bicuculline for one hour and treated with vehicle (DMSO), biotin-epoxomicin, or biotin-epoxomicin+Cycloheximide (CHX) in the last 10 minutes of the 1-hour stimulation. We chose bicuculline for our activity-inducing paradigm for these experiments since it provided us with more dynamic control of the timing of our experiments. Importantly, bicuculline stimulation recapitulates the earlier observations made using KCl-stimulation (FIG. 19D). Following these treatments in biological triplicates, proteins were extracted from the samples and derivatized using TMT tags following enzymatic digestion (FIG. 16A). In order to increase protein coverage, reduce artifacts from ratio compression, and increase our signal/noise ratio, peptides from all treatment groups fractionated offline before mass spectrometry (MS) analysis. We performed MS/MS analysis on each of the 24 fractions, with 2-hour runs per fraction in an Orbitrap Fusion Lumos mass spectrometer (FIG. 16A). An additional fragmentation event with high-energy collisional detection was used for quantification, which increases the accuracy of estimates of protein levels. Protein identification and TMT-based quantitation was conducted using Proteome Discoverer 2.1, applying a false discovery rate of 1% at the protein and peptide levels. Statistical normalization and analysis using inferential Bayes normalization to account for the population variance was performed as described in materials and methods. Statistically significant differences were determined after taking multiple comparisons testing into account. Overall, the combined analysis of the replicates across treatment groups yielded 141,295 peptides that were mapped to 8,223 proteins (FIG. 16B). The reproducibility across biological replicates was robust, with coefficients of variation of <10% observed for >99% of the proteins. We defined a co-translationally degraded substrate of the NMP as one with higher protein levels in bicuculline/biotin-epoxomicin-treated neurons as compared to both bicuculline and bicuculline/biotin-epoxomicin/CHX. Statistically significant differences between biotin-epoxomicin treated samples compared to the other groups were observed for 1,339 proteins at p<0.05, and 408 for p<0.01 (data not shown). However, we found it necessary to take multiple comparisons testing into account, increasing the stringency and robustness of this data set. This analysis yielded a list of 191 differentially expressed proteins, of which 122 were up-regulated, and therefore considered co-translationally degraded NMP substrates (FIGS. 16B,C).

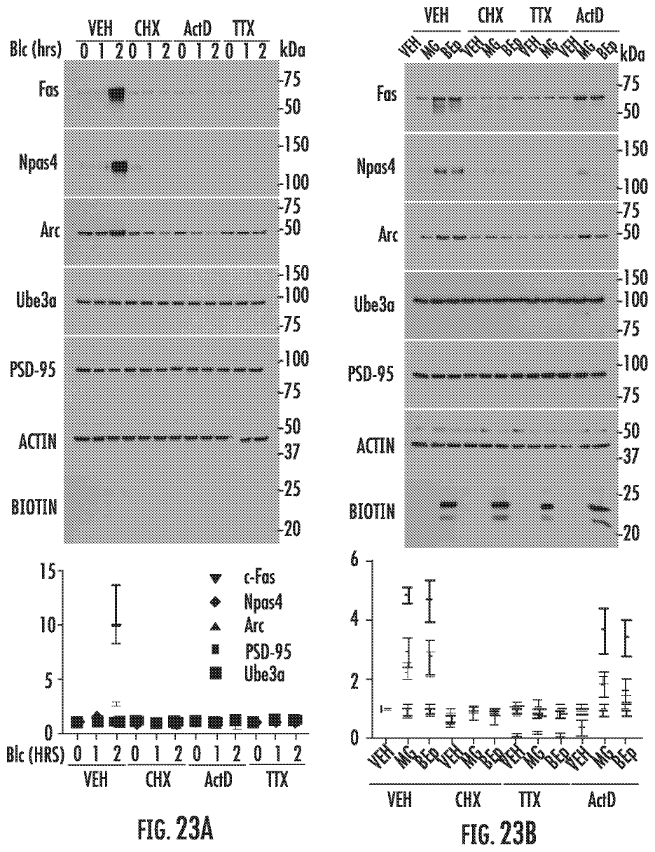

In our MS data, we identified NMP substrates that were previously described as ubiquitin-proteasome system (UPS) targets, such as Odcl and Rgs4 (FIG. 16D)(Asher et al., 2005; Bodenstein et al., 2007; Davydov and Varshaysky, 2000; Hoyt et al., 2003; Lee et al., 2005; Zhang et al., 2003). Further analysis of our MS data also revealed a set of substrates not previously shown to be turned over by proteasomes, such as Bex2, Ubc, and Snurf (FIG. 16D). However, by and large, the levels of many previously characterized UPS targets such as Shank, GKAP, PSD95, Ube3A and ApoER2 did not change in this assay (FIG. 16D) (Colledge et al., 2003; Ehlers, 2003; Gao et al., 2017; Hung et al., 2010; Lee et al., 2008; Shin et al., 2012). Further analysis of this dataset revealed an unusual enrichment of the immediate-early gene (IEG) products in our MS data as NMP substrates. These IEG proteins have all been shown to be activity-dependent targets of the UPS (Adler et al., 2010; Bae et al., 2002; Carle et al., 2007; Ito et al., 2005; Mabb et al., 2014; Peebles et al., 2010; Speckmann et al., 2016; Tsurumi et al., 1995). Specifically, we found that c-Fos, Fosb, Npas4, and Egr1 were significantly upregulated in response to biotin-epoxomicin treatment (FIG. 16D). These IEG proteins have characteristically low expression in unstimulated neurons and are induced by prolonged neuronal stimulation We initially attributed the upregulation observed in our MS data to canonical activity-induced mechanisms of IEG expression. However, by immunoblot analysis, bicuculline stimulation for one hour does not lead to significant increase in IEG protein expression (FIG. 23A). In contrast, following two hours of bicuculline stimulation, we observed the canonical induction of IEG protein expression that was dependent on neuronal activity, transcription, and translation (FIG. 23A) Based on these data, we suspected that our MS data revealed a unique mechanism of IEG protein regulation through the NMP, temporally distinct and prior to the canonical activity-dependent mechanisms of IEG protein expression.

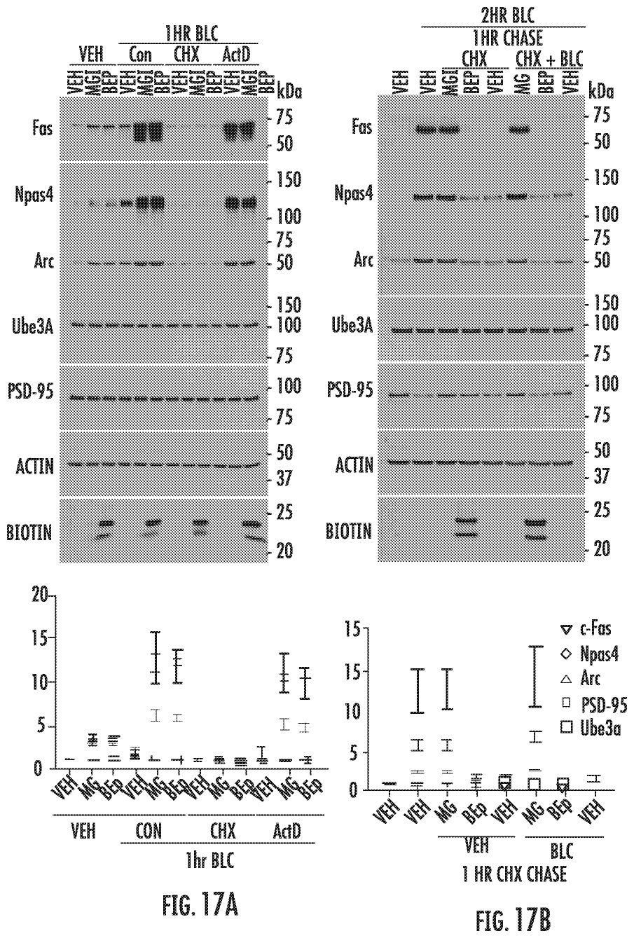

To independently validate our MS data, we used similar treatment conditions as in our MS analysis and analyzed IEG protein levels by immunoblot analysis. Neurons were stimulated with bicuculline for one hour, and treated with either MG-132 or biotin-epoxomicin for the final 10 minutes. The addition of either MG-132 or biotin-epoxomicin in the presence of bicuculline led to an accumulation of IEG proteins, but no change in the protein levels of UPS targets such as PSD95 or Ube3A (FIG. 17A). This increase in IEG protein levels was blocked by co-incubation with Cycloheximide, but transcriptional inhibitor actinomycin D had no effect (FIG. 17A). While we did not detect a change in Arc levels in the MS analysis, we did observe significant changes by immunoblot. This likely reflects the differences in detection sensitivity between the two methods. Notably, in the absence of bicuculline stimulation, MG-132 and biotin-epoxomicin treatment also led to a small, but reproducible increase in IEG products (FIGS. 17A and 23B). Addition of CHX or TTX blocked this inhibitor-mediated increase, suggesting that the effect depends on translation and baseline activity present in neuronal cultures (FIG. 23B). In all of these experiments, the effects on IEG protein expression due to treatment with MG-132 and biotin-epoxomicin were nearly identical, suggesting that the majority of changes we observe are due to the NMP, and not the cytosolic proteasome (FIGS. 17A and 23A). Together, we interpreted these data to mean that neuronal activity was required for and induces NMP-mediated degradation of IEG proteins.

Taking these experimental data together with the Markov modeling and validation, we hypothesized that the NMP exclusively mediates co-translational degradation of IEGs, and not full-length proteins. The data above demonstrating NMP-mediated IEG protein turnover do not distinguish between co-translational degradation and full-length protein degradation. To monitor turnover only of the full-length protein population, we took advantage of the robust induction of IEG protein expression following two hours of bicuculline stimulation (FIG. 23A). Following stimulation, we washed out the bicuculline to monitor the turnover of these IEG proteins for one hour. Neurons were incubated with Cycloheximide after the washout to prevent any further protein expression, allowing us to monitor the fate of these IEG protein products that had completed synthesis. As expected, we observed robust induction of immediate-early gene products following two hours of bicuculline stimulation that was largely turned over in one hour in the absence of sustained translation (FIG. 17B).This turnover was inhibited by the addition of MG-132, consistent with data from many groups demonstrating that IEG proteins are targeted by the ubiquitin-proteasome pathway (FIG. 17B) (Adler et al., 2010; Bae et al., 2002; Carle et al., 2007; Ito et al., 2005; Mabb et al., 2014; Peebles et al., 2010; Speckmann et al., 2016; Tsurumi et al., 1995). In contrast, biotin-epoxomicin does not prevent the turnover of these full-length IEG products (FIG. 17B). These data were the clearest demonstration that the NMP co-translationally degrades nascent polypeptides during states of activity, but is not capable of degrading a substrate once it is fully synthesized (FIG. 17B).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1 Coux, O., Tanaka, K. & Goldberg, A. L. Structure and functions of the 20S and 26S proteasomes. Annu Rev Biochem 65, 801-847, doi:10.1146/annurev.bi.65.070196.004101 (1996).
2 Ciechanover, A. The ubiquitin-proteasome pathway: on protein death and cell life. Embo J 17, 7151-7160, doi:10.1093/emboj/17.24.7151 (1998).
3 Ciechanover, A. & Schwartz, A. L. The ubiquitin-proteasome pathway: the complexity and myriad functions of proteins death. Proc Natl Acad Sci USA 95, 2727-2730 (1998).
4 Ben-Nissan, G. & Sharon, M. Regulating the 20S proteasome ubiquitin-independent degradation pathway. Biomolecules 4, 862-884, doi:10.3390/biom4030862 (2014).
5 Kisselev, A. F., van der Linden, W. A. & Overkleeft, H. S. Proteasome inhibitors: an expanding army attacking a unique target. Chem Biol 19, 99-115, doi:10.1016/j.chembio1.2012.01.003 (2012).
6 Ehlers, M. D. Activity level controls postsynaptic composition and signaling via the ubiquitin-proteasome system. Nat Neurosci 6, 231-242, doi:10.1038/nn1013 (2003).
7 Wang, H. R. et al. Regulation of cell polarity and protrusion formation by targeting RhoA for degradation. Science 302, 1775-1779, doi:10.1126/science.1090772 (2003).
8 Karpova, A., Mikhaylova, M., Thomas, U., Knopfel, T. & Behnisch, T. Involvement of protein synthesis and degradation in long-term potentiation of Schaffer collateral CA1 synapses. J Neurosci 26, 4949-4955, doi:10.1523/JNEUROSCI.4573-05.2006 (2006).
9 Dong, C., Upadhya, S. C., Ding, L., Smith, T. K. & Hegde, A. N. Proteasome inhibition enhances the induction and impairs the maintenance of late-phase long-term potentiation. Learn Mem 15, 335-347, doi:10.1101/1m.984508 (2008).
10 Djakovic, S. N., Schwarz, L. A., Barylko, B., DeMartino, G. N. & Patrick, G. N. Regulation of the proteasome by neuronal activity and calcium/calmodulin-dependent protein kinase II. J Biol Chem 284, 26655-26665, doi:10.1074/jbc.M109.021956 (2009).
11 Bingol, B. & Schuman, E. M. Activity-dependent dynamics and sequestration of proteasomes in dendritic spines. Nature 441, 1144-1148, doi:10.1038/nature04769 (2006).
12 Cai, F., Frey, J. U., Sanna, P. P. & Behnisch, T. Protein degradation by the proteasome is required for synaptic tagging and the heterosynaptic stabilization of hippocampal late-phase long-term potentiation. Neuroscience 169, 1520-1526, doi:10.1016/j.neuroscience.2010.06.032 (2010).
13 Rinetti, G. V. & Schweizer, F. E. Ubiquitination acutely regulates presynaptic neurotransmitter release in mammalian neurons. J Neurosci 30, 3157-3166, doi:10.1523/JNEUROSCI.3712-09.2010 (2010).
14 Wu, S. et al. Cellular calcium deficiency plays a role in neuronal death caused by proteasome inhibitors. J Neurochem 109, 1225-1236, doi:10.1111/j.1471-4159.2009.06037.x (2009).
15 Fonseca, R., Vabulas, R. M., Hard, F. U., Bonhoeffer, T. & Nagerl, U. V. A balance of protein synthesis and proteasome-dependent degradation determines the maintenance of LTP. Neuron 52, 239-245, doi:10.1016/j.neuron.2006.08.015 (2006).
16 Pines, J. & Lindon, C. Proteolysis: anytime, any place, anywhere? Nat Cell Biol 7, 731-735, doi:10.1038/ncb0805-731 (2005).
17 Asano, S. et al. Proteasomes. A molecular census of 26S proteasomes in intact neurons. Science 347, 439-442, doi:10.1126/science.1261197 (2015).
18 Patrick, G. N., Bingol, B., Weld, H. A. & Schuman, E. M. Ubiquitin-mediated proteasome activity is required for agonist-induced endocytosis of GluRs. Curr Biol 13, 2073-2081 (2003).
19 Blomen, V. A. et al. Gene essentiality and synthetic lethality in haploid human cells. Science 350, 1092-1096, doi:10.1126/science.aac7557 (2015).
20 van Weering, J. R. et al. Intracellular membrane traffic at high resolution. Methods Cell Biol 96, 619-648, doi:10.1016/S0091-679X(10)96026-3 (2010).
21 Chen, X. et al. PSD-95 family MAGUKs are essential for anchoring AMPA and NMDA receptor complexes at the postsynaptic density. Proc Natl Acad Sci USA 112, E6983-6992, doi:10.1073/pnas.1517045112 (2015).
22 Gazula, V. R. et al. Localization of Kv1.3 channels in presynaptic terminals of brainstem auditory neurons. J Comp Neurol 518, 3205-3220, doi:10.1002/cne.22393 (2010).
23 Kim, M. J., Dunah, A. W., Wang, Y. T. & Sheng, M. Differential roles of NR2A- and NR2B-containing NMDA receptors in Ras-ERK signaling and AMPA receptor trafficking. Neuron 46, 745-760, doi:10.1016/j.neuron.2005.04.031 (2005).
24 Hanley, J. G., Khatri, L., Hanson, P. I. & Ziff, E. B. NSF ATPase and alpha-/beta-SNAPs disassemble the AMPA receptor-PICK1 complex. Neuron 34, 53-67 (2002).
25 Peebles, C. L. et al. Arc regulates spine morphology and maintains network stability in vivo. Proc Natl Acad Sci USA 107, 18173-18178, doi:10.1073/pnas.1006546107 (2010).
26 Lin, D. T. et al. Regulation of AMPA receptor extrasynaptic insertion by 4.1N, phosphorylation and palmitoylation. Nat Neurosci 12, 879-887, doi:10.1038/nn.2351 (2009).
27 Ehlers, M. D. Reinsertion or degradation of AMPA receptors determined by activity-dependent endocytic sorting. Neuron 28, 511-525 (2000).
28 Caterina, M. J., Hereld, D. & Devreotes, P. N. Occupancy of the Dictyostelium cAMP receptor, cAR1, induces a reduction in affinity which depends upon COOH-terminal serine residues. J Biol Chem 270, 4418-4423 (1995).
29 Zhu, P. P. et al. Cellular localization, oligomerization, and membrane association of the hereditary spastic paraplegia 3A (SPG3A) protein atlastin. J Biol Chem 278, 49063-49071, doi:10.1074/jbc.M306702200 (2003).
30 Wunder, C., Lippincott-Schwartz, J. & Lorenz, H. Determining membrane protein topologies in single cells and high-throughput screening applications. Curr Protoc Cell Biol Chapter 5, Unit 5 7, doi:10.1002/0471143030.cb0507549 (2010).
31 Lee, Y. C., Srajer Gajdosik, M., Josic, D. & Lin, S. H. Plasma membrane isolation using immobilized concanavalin A magnetic beads. Methods Mol Biol 909, 29-41, doi:10.1007/978-1-61779-959-4_3 (2012).
32 Smith, M. J. & Koch, G. L. Multiple zones in the sequence of calreticulin (CRP55, calregulin, HACBP), a major calcium binding ER/SR protein. Embo J 8, 3581-3586 (1989).
33 Park, S. et al. GDE2 promotes neurogenesis by glycosylphosphatidylinositol-anchor cleavage of RECK. Science 339, 324-328, doi:10.1126/science.1231921 (2013).
34 Besche, H. C., Haas, W., Gygi, S. P. & Goldberg, A. L. Isolation of mammalian 26S proteasomes and p97/VCP complexes using the ubiquitin-like domain from HHR23B reveals novel proteasome-associated proteins. Biochemistry 48, 2538-2549, doi:10.1021/bi802198q (2009).
35 Werner, H., Dimou, L., Klugmann, M., Pfeiffer, S. & Nave, K. A. Multiple splice isoforms of proteolipid M6B in neurons and oligodendrocytes. Mol Cell Neurosci 18, 593-605, doi:10.1006/mcne.2001.1044 (2001).
36 Fuchsova, B., Fernandez, M. E., Alfonso, J. & Frasch, A. C. Cysteine residues in the large extracellular loop (EC2) are essential for the function of the stress-regulated glycoprotein M6a. J Biol Chem 284, 32075-32088, doi:10.1074/jbc.M109.012377 (2009).
37 Zhang, Y. et al. An RNA-sequencing transcriptome and splicing database of glia, neurons, and vascular cells of the cerebral cortex. J Neurosci 34, 11929-11947, doi:10.1523/JNEUROSCI.1860-14.2014 (2014).
38 Vilchez, D. et al. Increased proteasome activity in human embryonic stem cells is regulated by PSMD11. Nature 489, 304-308, doi:10.1038/nature11468 (2012).
39 Schubert, U. et al. Rapid degradation of a large fraction of newly synthesized proteins by proteasomes. Nature 404, 770-774, doi:10.1038/35008096 (2000).
40 Kisselev, A. F., Akopian, T. N. & Goldberg, A. L. Range of sizes of peptide products generated during degradation of different proteins by archaeal proteasomes. J Biol Chem 273, 1982-1989 (1998).
41 Li, N. et al. Relative quantification of proteasome activity by activity-based protein profiling and LC-MS/MS. Nat Protoc 8, 1155-1168, doi:10.1038/nprot.2013.065 (2013).
42 Meng, L. et al. Epoxomicin, a potent and selective proteasome inhibitor, exhibits in vivo antiinflammatory activity. Proc Natl Acad Sci USA 96, 10403-10408 (1999).
43 Patel, T. P., Man, K., Firestein, B. L. & Meaney, D. F. Automated quantification of neuronal networks and single-cell calcium dynamics using calcium imaging J Neurosci Methods 243, 26-38, doi:10.1016/j.jneumeth.2015.01.020 (2015).
44 Sato, Y., Watanabe, N., Fukushima, N., Mita, S. & Hirata, T. Actin-independent behavior and membrane deformation exhibited by the four-transmembrane protein M6a. PLoS One 6, e26702, doi:10.1371/journal.pone.0026702 (2011).
45 Besche, H. C. & Goldberg, A. L. Affinity purification of mammalian 26S proteasomes using an ubiquitin-like domain. Methods in molecular biology 832, 423-432, doi:10.1007/978-1-61779-474-2_29 (2012).
46 Tai, H. C. & Schuman, E. M. Ubiquitin, the proteasome and protein degradation in neuronal function and dysfunction. Nat Rev Neurosci 9, 826-838, doi:10.1038/nrn2499 (2008).
47 Tsvetkov, P. et al. Operational definition of intrinsically unstructured protein sequences based on susceptibility to the 20S proteasome. Proteins 70, 1357-1366, doi:10.1002/prot.21614 (2008).
48 Tsvetkov, P., Reuven, N., Prives, C. & Shaul, Y. Susceptibility of p53 unstructured N terminus to 20S proteasomal degradation programs the stress response. J Biol Chem 284, 26234-26242, doi:10.1074/jbc.M109.040493 (2009).
49 Schmidt, M. & Finley, D. Regulation of proteasome activity in health and disease. Biochim Biophys Acta 1843, 13-25, doi:10.1016/j.bbamcr.2013.08.012 (2014).
50 Tai, H. C. & Schuman, E M. Ubiquitin, the proteasome and protein degradation in neuronal function and dysfunction. Nature reviews. Neuroscience 9, 826-838, doi:10.1038/nrn2499 (2008).
51 Jiang, S., Dupont, N., Castillo, E. F. & Deretic, V. Secretory versus degradative autophagy:

unconventional secretion of inflammatory mediators. J Innate Immun 5, 471-479, doi:10.1159/000346707 (2013).
52 Lee, J. G., Takahama, S., Zhang, G., Tomarev, S. I. & Ye, Y. Unconventional secretion of misfolded proteins promotes adaptation to proteasome dysfunction in mammalian cells. Nat Cell Biol 18, 765-776, doi:10.1038/ncb3372 (2016).
53 Huh, G. S. et al. Functional requirement for class I MHC in CNS development and plasticity. Science 290, 2155-2159 (2000).
54 Shatz, C. J. MHC class I: an unexpected role in neuronal plasticity. Neuron 64, 40-45, doi:10.1016/j.neuron.2009.09.044 (2009).
55 Xia, Z., Dudek, H., Miranti, C. K. & Greenberg, M. E. Calcium influx via the NMDA receptor induces immediate early gene transcription by a MAP kinase/ERK-dependent mechanism. J Neurosci 16, 5425-5436 (1996).
56 Nicoll, R. A. & Roche, K. W. Long-term potentiation: peeling the onion. Neuropharmacology 74, 18-22, doi:10.1016/j.neuropharm.2013.02.010 (2013).
57 Malenka, R. C. & Nicoll, R. A. Long-term potentiation--a decade of progress? Science 285, 1870-1874 (1999).
58 Aakalu, G., Smith, W.B., Nguyen, N., Jiang, C., and Schuman, E.M. (2001). Dynamic visualization of local protein synthesis in hippocampal neurons. Neuron 30, 489-502.
59 Adler, J., Reuven, N., Kahana, C., and Shaul, Y. (2010). c-Fos proteasomal degradation is activated by a default mechanism, and its regulation by NAD(P)H:quinone oxidoreductase 1 determines c-Fos serum response kinetics. Mol Cell Biol 30, 3767-3778.
60 Alberts B, J. A., Lewis J, et al (2002). The Shape and Structure of Proteins. In Molecular Biology of the Cell (New York: Garland Science).
61 Anton, L. C., and Yewdell, J. W. (2014). Translating DRiPs: MHC class I immunosurveillance of pathogens and tumors. J Leukoc Biol 95, 551-562.
62 Asher, G., Bercovich, Z., Tsvetkov, P., Shaul, Y., and Kahana, C. (2005). 20S proteasomal degradation of ornithine decarboxylase is regulated by NQO1. Mol Cell 17, 645-655.
63 Bae, M. H., Jeong, C. H., Kim, S. H., Bae, M. K., Jeong, J. W., Ahn, M. Y., Bae, S. K., Kim, N. D., Kim, C. W., Kim, K. R., et al. (2002). Regulation of Egr-1 by association with the proteasome component C8. Biochim Biophys Acta 1592, 163-167.
64 Balchin, D., Hayer-Hartl, M., and Hartl, F. U. (2016). In vivo aspects of protein folding and quality control. Science 353, aac4354.
65 Ben-Nissan, G., and Sharon, M. (2014). Regulating the 20S proteasome ubiquitin-independent degradation pathway. Biomolecules 4, 862-884.
66 Benaroudj, N., Tarcsa, E., Cascio, P., and Goldberg, A. L. (2001). The unfolding of substrates and ubiquitin-independent protein degradation by proteasomes. Biochimie 83, 311-318.
67 Bengtson, M. H., and Joazeiro, C. A. (2010). Role of a ribosome-associated E3 ubiquitin ligase in protein quality control. Nature 467, 470-473.
68 Benoist, F., and Grand-Perret, T. (1997). Co-translational degradation of apolipoprotein B100 by the proteasome is prevented by microsomal triglyceride transfer protein. Synchronized translation studies on HepG2 cells treated with an inhibitor of microsomal triglyceride transfer protein. J Biol Chem 272, 20435-20442.
69 Bingol, B., and Schuman, E. M. (2006). Activity-dependent dynamics and sequestration of proteasomes in dendritic spines. Nature 441, 1144-1148.
70 Biran A., M. N., Adler J., Broennimann K , Reuven N., Shaul Y. (2017). A 20S proteasome receptor for degradation of intrinsically disordered proteins. bioRxiv.
71 Bodenstein, J., Sunahara, R. K., and Neubig, R. R. (2007). N-terminal residues control proteasomal degradation of RGS2, RGS4, and RGS5 in human embryonic kidney 293 cells. Mol Pharmacol 71, 1040-1050.
72 Brandman, O., Stewart-Ornstein, J., Wong, D., Larson, A., Williams, C. C., Li, G. W., Zhou, S., King, D., Shen, P. S., Weibezahn, J, et al. (2012). A ribosome-bound quality control complex triggers degradation of nascent peptides and signals translation stress. Cell 151, 1042-1054.
73 Carle, T. L., Ohnishi, Y. N., Ohnishi, Y. H., Alibhai, I. N., Wilkinson, M. B., Kumar, A., and Nestler, E. J. (2007). Proteasome-dependent and -independent mechanisms for FosB destabilization: identification of FosB degron domains and implications for DeltaFosB stability. Eur J Neurosci 25, 3009-3019.
74 Chu, J., Hong, N. A., Masuda, C. A., Jenkins, B. V., Nelms, K. A., Goodnow, C. C., Glynne, R. J., Wu, H., Masliah, E., Joazeiro, C. A., et al. (2009). A mouse forward genetics screen identifies LISTERIN as an E3 ubiquitin ligase involved in neurodegeneration. Proc Natl Acad Sci U S A 106, 2097-2103.
75 Ciechanover, A. (1998). The ubiquitin-proteasome pathway: on protein death and cell life. The EMBO journal 17, 7151-7160.
76 Colledge, M., Snyder, E. M., Crozier, R. A., Soderling, J. A., Jin, Y., Langeberg, L. K., Lu, H., Bear, M. F., and Scott, J. D. (2003). Ubiquitination regulates PSD-95 degradation and AMPA receptor surface expression. Neuron 40, 595-607.
77 Collins, G. A., and Goldberg, A. L. (2017). The Logic of the 26S Proteasome. Cell 169, 792-806.
78 Comyn, S. A., Chan, G. T., and Mayor, T. (2014). False start: cotranslational protein ubiquitination and cytosolic protein quality control. J Proteomics 100, 92-101.
79 Coux, O., Tanaka, K., and Goldberg, A. L. (1996). Structure and functions of the 20S and 26S proteasomes. Annual review of biochemistry 65, 801-847.
80 Davydov, I. V., and Varshaysky, A. (2000). RGS4 is arginylated and degraded by the N-end rule pathway in vitro. J Biol Chem 275, 22931-22941.
81 de Poot, S. A. H., Tian, G., and Finley, D. (2017). Meddling with Fate: The Proteasomal Deubiquitinating Enzymes. J Mol Biol 429, 3525-3545.
82 Deglincerti, A., Liu, Y., Colak, D., Hengst, U., Xu, G., and Jaffrey, S. R. (2015). Coupled local translation and degradation regulate growth cone collapse. Nat Commun 6, 6888.
83 Dieterich, D. C., Hodas, J. J., Gouzer, G., Shadrin, I. Y., Ngo, J. T., Triller, A., Tirrell, D. A., and Schuman, E. M. (2010). In situ visualization and dynamics of newly synthesized proteins in rat hippocampal neurons. Nat Neurosci 13, 897-905.
84 Dieterich, D. C., Link, A. J., Graumann, J., Tirrell, D. A., and Schuman, E. M. (2006). Selective identification of newly synthesized proteins in mammalian cells using bioorthogonal noncanonical amino acid tagging (BONCAT). Proc Natl Acad Sci U S A 103, 9482-9487.
85 Dimitrova, L. N., Kuroha, K., Tatematsu, T., and Inada, T. (2009). Nascent peptide-dependent translation arrest leads to Not4p-mediated protein degradation by the proteasome. J Biol Chem 284, 10343-10352.
86 Ding, Q., Dimayuga, E., Markesbery, W. R., and Keller, J. N. (2006). Proteasome inhibition induces reversible impairments in protein synthesis. FASEB J 20, 1055-1063.
87 Djakovic, S. N., Schwarz, L. A., Barylko, B., DeMartino, G. N., and Patrick, G. N. (2009). Regulation of the proteasome by neuronal activity and calcium/calmodulin-dependent protein kinase II. The Journal of biological chemistry 284, 26655-26665.
88 Duttler, S., Pechmann, S, and Frydman, J. (2013). Principles of cotranslational ubiquitination and quality control at the ribosome. Mol Cell 50, 379-393.
Ehlers, M. D. (2003). Activity level controls postsynaptic composition and signaling via the ubiquitin-proteasome system. Nature neuroscience 6, 231-242.
90 Etlinger, J. D., and Goldberg, A. L. (1977). A soluble ATP-dependent proteolytic system responsible for the degradation of abnormal proteins in reticulocytes. Proc Natl Acad Sci U S A 74, 54-58.
91 Finley, D., Ciechanover, A., and Varshaysky, A. (2004). Ubiquitin as a central cellular regulator. Cell 116, S29-32, 22 p following S32.
92 Flavell, S. W., Greenberg, M. E., 2008. Signaling mechanisms linking neuronal activity to gene expression and plasticity of the nervous system. Annu. Rev. Neurosci. 31, 563-90.
93 Fletcher, B. R., Hill, G. S., Long, J. M., Gallagher, M., Shapiro, M. L., and Rapp, P. R. (2014). A fine balance: Regulation of hippocampal Arc/Arg3.1 transcription, translation and degradation in a rat model of normal cognitive aging. Neurobiol Learn Mem 115, 58-67.
94 Fonseca, R., Nagerl, U. V., and Bonhoeffer, T. (2006a). Neuronal activity determines the protein synthesis dependence of long-term potentiation. Nat Neurosci 9, 478-480.
95 Fonseca, R., Vabulas, R. M., Hartl, F. U., Bonhoeffer, T., and Nagerl, U. V. (2006b). A balance of protein synthesis and proteasome-dependent degradation determines the maintenance of LTP. Neuron 52, 239-245.
96 Fortin, D. A., Davare, M. A., Srivastava, T., Brady, J. D., Nygaard, S., Derkach, V. A., and Soderling, T. R. (2010). Long-term potentiation-dependent spine enlargement requires synaptic Ca2+-permeable AMPA receptors recruited by CaM-kinase I. J Neurosci 30, 11565-11575.
97 Gao, J., Marosi, M., Choi, J., Achiro, J. M., Kim, S., Li, S., Otis, K., Martin, K. C., Portera-Cailliau, C., and Tontonoz, P. (2017). The E3 ubiquitin ligase IDOL regulates synaptic ApoER2 levels and is important for plasticity and learning. Elife 6.
98 Gloge, F., Becker, A. H., Kramer, G., and Bukau, B. (2014). Co-translational mechanisms of protein maturation. Curr Opin Struct Biol 24, 24-33.
99 Goldberg, A. L., and Dice, J. F. (1974). Intracellular protein degradation in mammalian and bacterial cells. Annu Rev Biochem 43, 835-869.
100 Ha, S. W., Ju, D., Hao, W., and Xie, Y. (2016). Rapidly Translated Polypeptides Are Preferred Substrates for Cotranslational Protein Degradation. J Biol Chem 291, 9827-9834.
101 Ha, S. W., Ju, D., and Xie, Y. (2014). Nuclear import factor Srp1 and its associated protein Sts1 couple ribosome-bound nascent polypeptides to proteasomes for cotranslational degradation. J Biol Chem 289, 2701-2710.
102 Haider, S., and Pal, R. (2013). Integrated analysis of transcriptomic and proteomic data. Curr Genomics 14, 91-110.
103 Hartl, F. U., Bracher, A., and Hayer-Hartl, M. (2011). Molecular chaperones in protein folding and proteostasis. Nature 475, 324-332.
104 Hartman, M. C., Josephson, K., and Szostak, J. W. (2006). Enzymatic aminoacylation of tRNA with unnatural amino acids. Proc Natl Acad Sci U S A 103, 4356-4361.
105 Hoyt, M. A., Zhang, M., and Coffino, P. (2003). Ubiquitin-independent mechanisms of mouse ornithine decarboxylase degradation are conserved between mammalian and fungal cells. J Biol Chem 278, 12135-12143.
106 Hung, A. Y., Sung, C. C., Brito, I. L., and Sheng, M. (2010). Degradation of postsynaptic scaffold GKAP and regulation of dendritic spine morphology by the TRIM3 ubiquitin ligase in rat hippocampal neurons. PLoS One 5, e9842.
107 Inada, T. (2017). The Ribosome as a Platform for mRNA and Nascent Polypeptide Quality Control. Trends Biochem Sci 42, 5-15.
108 Ito, K., Chadani, Y., Nakamori, K., Chiba, S., Akiyama, Y., and Abo, T. (2011). Nascentome analysis uncovers futile protein synthesis in Escherichia coli. PLoS One 6, e28413.
109 Ito, Y., Inoue, D., Kido, S., and Matsumoto, T. (2005). c-Fos degradation by the ubiquitin-proteasome proteolytic pathway in osteoclast progenitors. Bone 37, 842-849.
110 Kammers, K., Cole, R. N., Tiengwe, C., and Ruczinski, I. (2015). Detecting Significant Changes in Protein Abundance. EuPA Open Proteom 7, 11-19.
111 Kelleher, R. J., 3rd, Govindarajan, A., and Tonegawa, S. (2004). Translational regulatory mechanisms in persistent forms of synaptic plasticity. Neuron 44, 59-73.
112 Kirstein-Miles, J., Scior, A., Deuerling, E., and Morimoto, R. I. (2013). The nascent polypeptide-associated complex is a key regulator of proteostasis. EMBO J 32, 1451-1468.
113 Klein, M. E., Castillo, P. E., and Jordan, B. A. (2015). Coordination between Translation and Degradation Regulates Inducibility of mGluR-LTD. Cell Rep.
114 Kramer, G., Boehringer, D., Ban, N., and Bukau, B. (2009). The ribosome as a platform for co-translational processing, folding and targeting of newly synthesized proteins. Nat Struct Mol Biol 16, 589-597.
115 Landgraf, P., Antileo, E. R., Schuman, E. M., and Dieterich, D. C. (2015). BONCAT: metabolic labeling, click chemistry, and affinity purification of newly synthesized proteomes. Methods Mol Biol 1266, 199-215.
116 Lane, T. J., and Pande, V. S. (2013). Inferring the rate-length law of protein folding. PLoS One 8, e78606.
117 Lee, M. J., Tasaki, T., Moroi, K., An, J. Y., Kimura, S., Davydov, I. V., and Kwon, Y. T. (2005). RGS4 and RGSS are in vivo substrates of the N-end rule pathway. Proc Natl Acad Sci U S A 102, 15030-15035.
118 Lee, S. H., Choi, J. H., Lee, N., Lee, H. R., Kim, J. I., Yu, N. K., Choi, S. L., Lee, S. H., Kim, H., and Kaang, B. K. (2008). Synaptic protein degradation underlies destabilization of retrieved fear memory. Science 319, 1253-1256.
119 Li, N., Kuo, C. L., Paniagua, G., van den Elst, H., Verdoes, M., Willems, L. I., van der Linden, W. A., Ruben, M., van Genderen, E., Gubbens, J., et al. (2013). Relative quantification of proteasome activity by activity-based protein profiling and LC-MS/MS. Nature protocols 8, 1155-1168.
120 Lin, Y., Bloodgood, B. L., Hauser, J. L., Lapan, A. D., Koon, A. C., Kim, T. K., Hu, L. S., Malik, A. N., and Greenberg, M. E. (2008). Activity-dependent regulation of inhibitory synapse development by Npas4. Nature 455, 1198-1204.
121 Mabb, A. M., Je, H. S., Wall, M. J., Robinson, C. G., Larsen, R. S., Qiang, Y., Correa, S. A., and Ehlers, M. D. (2014). Triad3A regulates synaptic strength by ubiquitination of Arc. Neuron 82, 1299-1316.
122 Maier, T., Guell, M., and Serrano, L. (2009). Correlation of mRNA and protein in complex biological samples. FEBS Lett 583, 3966-3973.
123 Margolis, S. S., Salogiannis, J, Lipton, D. M., Mandel-Brehm, C., Wills, Z. P., Mardinly, A. R., Hu, L., Greer, P. L., Bikoff, J. B., Ho, H. Y., et al. (2010). EphB-mediated degradation of the RhoA GEF Ephexin5 relieves a developmental brake on excitatory synapse formation. Cell 143, 442-455.
124 Marin, P., Nastiuk, K. L., Daniel, N., Girault, J. A., Czernik, A. J., Glowinski, J., Nairn, A. C., and Premont, J. (1997). Glutamate-dependent phosphorylation of elongation factor-2 and inhibition of protein synthesis in neurons. J Neurosci 17, 3445-3454.
125 McShane, E., Sin, C., Zauber, H., Wells, J. N., Donnelly, N., Wang, X., Hou, J., Chen, W., Storchova, Z., Marsh, J. A., et al. (2016). Kinetic Analysis of Protein Stability Reveals Age-Dependent Degradation. Cell 167, 803-815 e821.
126 Meng, L., Mohan, R., Kwok, B. H., Elofsson, M., Sin, N., and Crews, C. M. (1999a). Epoxomicin, a potent and selective proteasome inhibitor, exhibits in vivo antiinflammatory activity. Proceedings of the National Academy of Sciences of the United States of America 96, 10403-10408.
127 Meng, L., Mohan, R., Kwok, B. H., Elofsson, M., Sin, N., and Crews, C. M. (1999b). Epoxomicin, a potent and selective proteasome inhibitor, exhibits in vivo antiinflammatory activity. Proc Natl Acad Sci U S A 96, 10403-10408.
128 Nathans, D. (1964). Puromycin Inhibition of Protein Synthesis: Incorporation of Puromycin into Peptide Chains Proc Natl Acad Sci U S A 51, 585-592.
129 Nathans, D., and Neidle, A. (1963). Structural requirements for puromycin inhibition of protein synthesis. Nature 197, 1076-1077.
130 Obeng, E. A., Carlson, L. M., Gutman, D. M., Harrington, W. J., Jr., Lee, K. P., and Boise, L. H. (2006). Proteasome inhibitors induce a terminal unfolded protein response in multiple myeloma cells. Blood 107, 4907-4916.
131 Ostroff, L. E., Botsford, B., Gindina, S., Cowansage, K. K., LeDoux, J. E., Klann, E, and Hoeffer, C. (2017). Accumulation of Polyribosomes in Dendritic Spine Heads, But Not Bases and Necks, during Memory Consolidation Depends on Cap-Dependent Translation Initiation. J Neurosci 37, 1862-1872.
132 Ostroff, L. E., Fiala, J. C., Allwardt, B., and Harris, K. M. (2002). Polyribosomes redistribute from dendritic shafts into spines with enlarged synapses during LTP in developing rat hippocampal slices. Neuron 35, 535-545.
133 Pande, V. S. (2014). Understanding protein folding using Markov state models. Adv Exp Med Biol 797, 101-106.
134 Pechmann, S , Willmund, F., and Frydman, J. (2013). The ribosome as a hub for protein quality control. Mol Cell 49, 411-421.
135 Peebles, C. L., Yoo, J., Thwin, M. T., Palop, J. J., Noebels, J. L., and Finkbeiner, S. (2010). Arc regulates spine morphology and maintains network stability in vivo. Proc Natl Acad Sci U S A 107, 18173-18178.
136 Prouty, W. F., and Goldberg, A. L. (1972). Fate of abnormal proteins in E. coli accumulation in intracellular granules before catabolism. Nat New Biol 240, 147-150.
137 Prouty, W. F., Karnovsky, M. J., and Goldberg, A. L. (1975). Degradation of abnormal proteins in Escherichia coli. Formation of protein inclusions in cells exposed to amino acid analogs. J Biol Chem 250, 1112-1122.
138 Ramachandran, K. V., and Margolis, S. S. (2017). A mammalian nervous-system-specific plasma membrane proteasome complex that modulates neuronal function. Nat Struct Mol Biol 24, 419-430.
139 Robertson, J. H., and Wheatley, D. N. (1979). Pools and protein synthesis in mammalian cells. Biochem J 178, 699-709.
140 Rock, K. L., Farfan-Arribas, D. J., Colbert, J. D., and Goldberg, A. L. (2014). Re-examining class-I presentation and the DRIP hypothesis. Trends Immunol 35, 144-152.
141 Rock, K. L., Gramm, C., Rothstein, L., Clark, K., Stein, R., Dick, L., Hwang, D., and Goldberg, A. L. (1994). Inhibitors of the proteasome block the degradation of most cell proteins and the generation of peptides presented on MHC class I molecules. Cell 78, 761-771.
142 Scheetz, A. J., Nairn, A. C., and Constantine-Paton, M. (2000). NMDA receptor-mediated control of protein synthesis at developing synapses. Nat Neurosci 3, 211-216.
143 Schmidt, M., and Finley, D. (2014). Regulation of proteasome activity in health and disease. Biochimica et biophysica acta 1843, 13-25.
144 Schratt, G. M., Nigh, E. A., Chen, W. G., Hu, L., and Greenberg, M. E. (2004). BDNF regulates the translation of a select group of mRNAs by a mammalian target of rapamycin-phosphatidylinositol 3-kinase-dependent pathway during neuronal development. J Neurosci 24, 7366-7377.
145 Schubert, U., Anton, L. C., Gibbs, J., Norbury, C. C., Yewdell, J. W., and Bennink, J. R. (2000). Rapid degradation of a large fraction of newly synthesized proteins by proteasomes. Nature 404, 770-774.
146 Schwanhausser, B., Busse, D., Li, N., Dittmar, G., Schuchhardt, J., Wolf, J., Chen, W., and Selbach, M. (2011). Global quantification of mammalian gene expression control. Nature 473, 337-342.
147 Schwanhausser, B., Wolf, J., Selbach, M., and Busse, D. (2013). Synthesis and degradation jointly determine the responsiveness of the cellular proteome. Bioessays 35, 597-601.
148 Shao, S., von der Malsburg, K., and Hegde, R. S. (2013). Listerin-dependent nascent protein ubiquitination relies on ribosome subunit dissociation. Mol Cell 50, 637-648.
149 Shin, S. M., Zhang, N., Hansen, J., Gerges, N. Z., Pak, D. T., Sheng, M., and Lee, S. H. (2012). GKAP orchestrates activity-dependent postsynaptic protein remodeling and homeostatic scaling. Nat Neurosci 15, 1655-1666.
150 Sin, N., Kim, K. B., Elofsson, M., Meng, L., Auth, H., Kwok, B. H., and Crews, C. M. (1999). Total synthesis of the potent proteasome inhibitor epoxomicin: a useful tool for understanding proteasome biology. Bioorg Med Chem Lett 9, 2283-2288.
151 Sontag, E. M., Samant, R. S., and Frydman, J (2017). Mechanisms and Functions of Spatial Protein Quality Control. Annu Rev Biochem 86, 97-122.
152 Speckmann, T, Sabatini, P. V., Nian, C., Smith, R. G., and Lynn, F. C. (2016). Npas4 Transcription Factor Expression Is Regulated by Calcium Signaling Pathways and Prevents Tacrolimus-induced Cytotoxicity in Pancreatic Beta Cells. J Biol Chem 291, 2682-2695.
153 Tai, H. C., Besche, H., Goldberg, A. L., and Schuman, E. M. (2010). Characterization of the Brain 26S Proteasome and its Interacting Proteins. Front Mol Neurosci 3.
154 Tcherkezian, J., Brittis, P. A., Thomas, F., Roux, P. P., and Flanagan, J. G. (2010). Transmembrane receptor DCC associates with protein synthesis machinery and regulates translation. Cell 141, 632-644.
155 Tsurumi, C., Ishida, N., Tamura, T., Kakizuka, A., Nishida, E., Okumura, E., Kishimoto, T., Inagaki, M., Okazaki, K., Sagata, N., et al. (1995). Degradation of c-Fos by the 26S proteasome is accelerated by c-Jun and multiple protein kinases. Mol Cell Biol 15, 5682-5687.
156 Tsvetkov, P., Asher, G., Paz, A., Reuven, N., Sussman, J. L., Silman, I., and Shaul, Y. (2008). Operational definition of intrinsically unstructured protein sequences based on susceptibility to the 20S proteasome. Proteins 70, 1357-1366.
157 Tsvetkov, P., Reuven, N., and Shaul, Y. (2009). The nanny model for IDPs. Nat Chem Biol 5, 778-781.
158 Turner, G. C., and Varshaysky, A. (2000). Detecting and measuring cotranslational protein degradation in vivo. Science 289, 2117-2120.
159 Vabulas, R. M., and Hard, F. U. (2005). Protein synthesis upon acute nutrient restriction relies on proteasome function. Science 310, 1960-1963.
160 von der Malsburg, K., Shao, S., and Hegde, R. S. (2015). The ribosome quality control pathway can access nascent polypeptides stalled at the Sec61 translocon. Mol Biol Cell 26, 2168-2180.
161 Wang, F., Durfee, L. A., and Huibregtse, J. M. (2013). A cotranslational ubiquitination pathway for quality control of misfolded proteins. Mol Cell 50, 368-378.
162 West, A. E., Chen, W. G., Dalva, M. B., Dolmetsch, R. E., Kornhauser, J. M., Shaywitz, A. J., Takasu, M. A., Tao, X., and Greenberg, M. E. (2001). Calcium regulation of neuronal gene expression. Proc Natl Acad Sci U S A 98, 11024-11031.
163 West, A. E., and Greenberg, M. E. (2011). Neuronal activity-regulated gene transcription in synapse development and cognitive function. Cold Spring Harb Perspect Biol 3.
164 Wheatley, D. N. (2011). Protein balance: a fundamental question of cell biology needing reappraisal. Cell Biol Int 35, 453-455.
165 Wheatley, D. N., Giddings, M. R., and Inglis, M. S. (1980). Kinetics of degradation of “short-” and “long-lived” proteins in cultured mammalian cells. Cell Biol Int Rep 4, 1081-1090.
166 Wheatley, D. N., Grisolia, S., and Hernandez-Yago, J. (1982). Significance of the rapid degradation of newly synthesized proteins in mammalian cells: a working hypothesis. J Theor Biol 98, 283-300.
167 Wheatley, D. N., and Inglis, M. S. (1980). An intracellular perfusion system linking pools and protein synthesis. J Theor Biol 83, 437-445.
168 Wu, B., Eliscovich, C., Yoon, Y. J., and Singer, R. H. (2016). Translation dynamics of single mRNAs in live cells and neurons. Science 352, 1430-1435.
169 Wu, W. K., Volta, V., Cho, C. H., Wu, Y. C., Li, H. T., Yu, L., Li, Z. J., and Sung, J. J. (2009). Repression of protein translation and mTOR signaling by proteasome inhibitor in colon cancer cells. Biochem Biophys Res Commun 386, 598-601.
170 Xia, Z., Dudek, H., Miranti, C. K., and Greenberg, M. E. (1996). Calcium influx via the NMDA receptor induces immediate early gene transcription by a MAP kinase/ERK-dependent mechanism. J Neurosci 16, 5425-5436.
171 Yonashiro, R., Tahara, E. B., Bengtson, M. H., Khokhrina, M., Lorenz, H., Chen, K. C., Kigoshi-Tansho, Y., Savas, J. N., Yates, J. R., Kay, S. A., et al. (2016). The Rqc2/Tae2 subunit of the ribosome-associated quality control (RQC) complex marks ribosome-stalled nascent polypeptide chains for aggregation. Elife 5, el 1794.
172 Zhang, M., Pickart, C. M., and Coffino, P. (2003). Determinants of proteasome recognition of ornithine decarboxylase, a ubiquitin-independent substrate. EMBO J 22, 1488-1496.
173 Zhao, J., Garcia, G. A., and Goldberg, A. L. (2016). Control of proteasomal proteolysis by mTOR. Nature 529, E1-2.

1. (canceled)

2. A method for inhibiting neuronal activity or cognitive function in a subject comprising administering to the subject an effective amount of a neural membrane bound proteasome (NMP) inhibitor, wherein the inhibitor is selected from the group consisting of peptide aldehydes, peptide boronates, and nonpeptide inhibitors.

3. (canceled)

4. The method of claim 2, wherein the NMP inhibitor is a proteasome inhibitor selected from the group consisting of Epoxomicin, Lactacystin, Bortezomib, MG-132, Carfilzomib, MLN9708, Ixazomib, PI-1840, ONX-0914, Oprozomib, CEP-18770, and Gabexate Mesylate.

5. (canceled)

6. The method of claim 2, for use in modulating an NMP associated disease or disorder of neuronal cells in a subject.

7. (canceled)

8. The method of claim 6, wherein the NMP inhibitor is a proteasome inhibitor selected from the group consisting of Epoxomicin, Lactacystin, Bortezomib, MG-132, Carfilzomib, MLN9708, Ixazomib, PI-1840, ONX-0914, Oprozomib, CEP-18770, and Gabexate Mesylate.

9. The method of claim 6, wherein the NMP associated disease is selected from the groups consisting of epilepsy, encephalopathy, seizures due to brain tumors, chronic pain, Parkinson's disease, Huntington's disease, Alzheimer's disease, neurodegenerative diseases, and other muscle spasm disorders.

10. (canceled)

11. A method for stimulating or enhancing neuronal activity or cognitive function in a subject comprising administering to the subject, an effective amount of a composition comprising secreted, neuronal activity-induced, proteasomal peptides (SNAPPs).

12. The method of claim 11, wherein the SNAPPs have a molecular weight between 500 to 3000 Daltons.

13. The method of claim 11, wherein the SNAPPs are derived from a neuron selected from the group consisting of cortical, hippocampal, cerebellar, motor, sensory,

14. The method of claim 11, wherein the SNAPPs comprise at least one detectable moiety as an imaging agent.

15. The method of claim 11, wherein the SNAPPs comprise at least one detectable moiety as a radionuclide.

16. The method of claim 14, wherein the at least one detectable moiety is covalently attached to the SNAPPs via a biotinylated linker molecule.

17. The method of claim 11, wherein the subject is suffering from Alzheimer's disease or dementia.

18. The composition method of claim 11, wherein the composition further comprises an effective amount of at least one additional biologically active agent.
